SEQUENCE LISTING (536 sequences)

SEQ ID NO: 1 - DNA, 22 bp, Artificial Sequence
  Description of Artificial Sequence Synthetic oligonucleotide
acactgtcgtcgaatggccact

SEQ ID NO: 2 - DNA, 22 bp, Artificial Sequence
  Description of Artificial Sequence Synthetic oligonucleotide
cacactgtcgtcgaatggccac

SEQ ID NO: 3 - DNA, 22 bp, Artificial Sequence
  Description of Artificial Sequence Synthetic oligonucleotide
atacatccatggctaatgaatt

SEQ ID NO: 4 - DNA, 22 bp, Artificial Sequence
  Description of Artificial Sequence Synthetic oligonucleotide
aatacatccatggctaatgaat

SEQ ID NO: 5 - DNA, 22 bp, Artificial Sequence
  Description of Artificial Sequence Synthetic oligonucleotide
tgtctttcctgctgcttctgcc

SEQ ID NO: 6 - DNA, 22 bp, Artificial Sequence
  Description of Artificial Sequence Synthetic oligonucleotide
ttgtctttcctgctgcttctgc

SEQ ID NO: 7 - DNA, 22 bp, Artificial Sequence
  Description of Artificial Sequence Synthetic oligonucleotide
tcttctcagccactgttgccac

SEQ ID NO: 8 - DNA, 22 bp, Artificial Sequence
  Description of Artificial Sequence Synthetic oligonucleotide
ggtcttctcagccactgttgcc

SEQ ID NO: 9 - DNA, 22 bp, Artificial Sequence
  Description of Artificial Sequence Synthetic oligonucleotide
gctctttggtcttctcagccac

SEQ ID NO: 10 - DNA, 22 bp, Artificial Sequence
  Description of Artificial Sequence Synthetic oligonucleotide
tcacttgctctttggtcttctc

SEQ ID NO: 11 - DNA, 22 bp, Artificial Sequence
  Description of Artificial Sequence Synthetic oligonucleotide
tttgtcacttgctctttggtct

SEQ ID NO: 12 - DNA, 22 bp, Artificial Sequence
  Description of Artificial Sequence Synthetic oligonucleotide
tgtcttctgggctactgctgtc

SEQ ID NO: 13 - DNA, 22 bp, Artificial Sequence
  Description of Artificial Sequence Synthetic oligonucleotide
ccagtggctgctgcaatgctcc

SEQ ID NO: 14 - DNA, 22 bp, Artificial Sequence
  Description of Artificial Sequence Synthetic oligonucleotide
agaattccttcctgtggggctc

SEQ ID NO: 15 - DNA, 22 bp, Artificial Sequence
  Description of Artificial Sequence Synthetic oligonucleotide
atatcttccagaattccttcct

SEQ ID NO: 16 - DNA, 22 bp, Artificial Sequence
  Description of Artificial Sequence Synthetic oligonucleotide
catatcttccagaattccttcc

SEQ ID NO: 17 - DNA, 22 bp, Artificial Sequence
  Description of Artificial Sequence Synthetic oligonucleotide
gcatatcttccagaattccttc

SEQ ID NO: 18 - DNA, 22 bp, Artificial Sequence
  Description of Artificial Sequence Synthetic oligonucleotide
aggatccacaggcatatcttcc

SEQ ID NO: 19 - DNA, 22 bp, Artificial Sequence
  Description of Artificial Sequence Synthetic oligonucleotide
ctcattgtcaggatccacaggc

SEQ ID NO: 20 - DNA, 22 bp, Artificial Sequence
  Description of Artificial Sequence Synthetic oligonucleotide
cataagcctcattgtcaggatc

SEQ ID NO: 21 - DNA, 22 bp, Artificial Sequence
  Description of Artificial Sequence Synthetic oligonucleotide
tcataagcctcattgtcaggat

SEQ ID NO: 22 - DNA, 22 bp, Artificial Sequence
  Description of Artificial Sequence Synthetic oligonucleotide
ctcagaaggcatttcataagcc

SEQ ID NO: 23 - DNA, 22 bp, Artificial Sequence
  Description of Artificial Sequence Synthetic oligonucleotide
cgtagtcttgatacccttcctc

SEQ ID NO: 24 - DNA, 22 bp, Artificial Sequence
  Description of Artificial Sequence Synthetic oligonucleotide
tcgtagtcttgatacccttcct

SEQ ID NO: 25 - DNA, 22 bp, Artificial Sequence
  Description of Artificial Sequence Synthetic oligonucleotide
caggttcgtagtcttgataccc

SEQ ID NO: 26 - DNA, 22 bp, Artificial Sequence
  Description of Artificial Sequence Synthetic oligonucleotide
atttcttaggcttcaggttcgt

SEQ ID NO: 27 - DNA, 22 bp, Artificial Sequence
  Description of Artificial Sequence Synthetic oligonucleotide
atatttcttaggcttcaggttc

SEQ ID NO: 28 - DNA, 22 bp, Artificial Sequence
  Description of Artificial Sequence Synthetic oligonucleotide
agatatttcttaggcttcaggt

SEQ ID NO: 29 - DNA, 22 bp, Artificial Sequence
  Description of Artificial Sequence Synthetic oligonucleotide
cagatctcaagaaactgggagc

SEQ ID NO: 30 - DNA, 22 bp, Artificial Sequence
  Description of Artificial Sequence Synthetic oligonucleotide
ctgtcagcagatctcaagaaac

SEQ ID NO: 31 - DNA, 22 bp, Artificial Sequence
  Description of Artificial Sequence Synthetic oligonucleotide
tcatgactgggcacattggaac

SEQ ID NO: 32 - DNA, 22 bp, Artificial Sequence
  Description of Artificial Sequence Synthetic oligonucleotide
gtacagatacttcaatcactgc

SEQ ID NO: 33 - DNA, 22 bp, Artificial Sequence
  Description of Artificial Sequence Synthetic oligonucleotide
agtgaaagggaagcaccgaaat

SEQ ID NO: 34 - DNA, 22 bp, Artificial Sequence
  Description of Artificial Sequence Synthetic oligonucleotide
taacatcgtagattgaagccac

SEQ ID NO: 35 - DNA, 22 bp, Artificial Sequence
  Description of Artificial Sequence Synthetic oligonucleotide
tttaacatcgtagattgaagcc

SEQ ID NO: 36 - DNA, 22 bp, Artificial Sequence
  Description of Artificial Sequence Synthetic oligonucleotide
tcagttcttaattcatgttgct

SEQ ID NO: 37 - DNA, 22 bp, Artificial Sequence
  Description of Artificial Sequence Synthetic oligonucleotide
gtcagttcttaattcatgttgc

SEQ ID NO: 38 - DNA, 22 bp, Artificial Sequence
  Description of Artificial Sequence Synthetic oligonucleotide
acttaaggaaccagtgcatacc

SEQ ID NO: 39 - DNA, 22 bp, Artificial Sequence
  Description of Artificial Sequence Synthetic oligonucleotide
aatcacagccacttaaggaacc

SEQ ID NO: 40 - DNA, 22 bp, Artificial Sequence
  Description of Artificial Sequence Synthetic oligonucleotide
taatcacagccacttaaggaac

SEQ ID NO: 41 - DNA, 22 bp, Artificial Sequence
  Description of Artificial Sequence Synthetic oligonucleotide
tcaataattaatcacagccact

SEQ ID NO: 42 - DNA, 22 bp, Artificial Sequence
  Description of Artificial Sequence Synthetic oligonucleotide
ttcaataattaatcacagccac

SEQ ID NO: 43 - DNA, 22 bp, Artificial Sequence
  Description of Artificial Sequence Synthetic oligonucleotide
ctttcaataattaatcacagcc

SEQ ID NO: 44 - DNA, 22 bp, Artificial Sequence
  Description of Artificial Sequence Synthetic oligonucleotide
tacaatagtagttggggtcttc

SEQ ID NO: 45 - DNA, 22 bp, Artificial Sequence
  Description of Artificial Sequence Synthetic oligonucleotide
attgaagggagaaatagaccac

SEQ ID NO: 46 - DNA, 22 bp, Artificial Sequence
  Description of Artificial Sequence Synthetic oligonucleotide
tgacaggattgaagggagaaat

SEQ ID NO: 47 - DNA, 22 bp, Artificial Sequence
  Description of Artificial Sequence Synthetic oligonucleotide
gtcagaaaggtacagcattcac

SEQ ID NO: 48 - DNA, 22 bp, Artificial Sequence
  Description of Artificial Sequence Synthetic oligonucleotide
ttgtcagaaaggtacagcattc

SEQ ID NO: 49 - DNA, 22 bp, Artificial Sequence
  Description of Artificial Sequence Synthetic oligonucleotide
attgtcagaaaggtacagcatt

SEQ ID NO: 50 - DNA, 22 bp, Artificial Sequence
  Description of Artificial Sequence Synthetic oligonucleotide
tattgtcagaaaggtacagcat

SEQ ID NO: 51 - DNA, 22 bp, Artificial Sequence
  Description of Artificial Sequence Synthetic oligonucleotide
tcttctacactgcttagttccc

SEQ ID NO: 52 - DNA, 22 bp, Artificial Sequence
  Description of Artificial Sequence Synthetic oligonucleotide
tgactctggtagttccaacgat

SEQ ID NO: 53 - DNA, 22 bp, Artificial Sequence
  Description of Artificial Sequence Synthetic oligonucleotide
aggtgactctggtagttccaac

SEQ ID NO: 54 - DNA, 22 bp, Artificial Sequence
  Description of Artificial Sequence Synthetic oligonucleotide
ttaaggtgactctggtagttcc

SEQ ID NO: 55 - DNA, 22 bp, Artificial Sequence
  Description of Artificial Sequence Synthetic oligonucleotide
tttaaaggaggccatgaaattt

SEQ ID NO: 56 - DNA, 22 bp, Artificial Sequence
  Description of Artificial Sequence Synthetic oligonucleotide
tcctagaattcatatatttggc

SEQ ID NO: 57 - DNA, 22 bp, Artificial Sequence
  Description of Artificial Sequence Synthetic oligonucleotide
tgaatacatataaactgctagc

SEQ ID NO: 58 - DNA, 22 bp, Artificial Sequence
  Description of Artificial Sequence Synthetic oligonucleotide
ctcatgaatacatataaactgc

SEQ ID NO: 59 - DNA, 22 bp, Artificial Sequence
  Description of Artificial Sequence Synthetic oligonucleotide
actcattcctccttccttcctc

SEQ ID NO: 60 - DNA, 22 bp, Artificial Sequence
  Description of Artificial Sequence Synthetic oligonucleotide
tcactcattcctccttccttcc

SEQ ID NO: 61 - DNA, 22 bp, Artificial Sequence
  Description of Artificial Sequence Synthetic oligonucleotide
gtcactcattcctccttccttc

SEQ ID NO: 62 - DNA, 22 bp, Artificial Sequence
  Description of Artificial Sequence Synthetic oligonucleotide
cactctgtagtagtctctcttc

SEQ ID NO: 63 - DNA, 22 bp, Artificial Sequence
  Description of Artificial Sequence Synthetic oligonucleotide
cttagcactctgtagtagtctc

SEQ ID NO: 64 - DNA, 22 bp, Artificial Sequence
  Description of Artificial Sequence Synthetic oligonucleotide
acttaccatttctctctagtgt

SEQ ID NO: 65 - DNA, 22 bp, Artificial Sequence
  Description of Artificial Sequence Synthetic oligonucleotide
tcctaaccgccactttctaacc

SEQ ID NO: 66 - DNA, 22 bp, Artificial Sequence
  Description of Artificial Sequence Synthetic oligonucleotide
atatatcctaaccgccactttc

SEQ ID NO: 67 - DNA, 22 bp, Artificial Sequence
  Description of Artificial Sequence Synthetic oligonucleotide
aatatatcctaaccgccacttt

SEQ ID NO: 68 - DNA, 22 bp, Artificial Sequence
  Description of Artificial Sequence Synthetic oligonucleotide
aaatatatcctaaccgccactt

SEQ ID NO: 69 - DNA, 22 bp, Artificial Sequence
  Description of Artificial Sequence Synthetic oligonucleotide
aatatgctgctttaggtagatt

SEQ ID NO: 70 - DNA, 22 bp, Artificial Sequence
  Description of Artificial Sequence Synthetic oligonucleotide
tctagttctgtcctctatttct

SEQ ID NO: 71 - DNA, 22 bp, Artificial Sequence
  Description of Artificial Sequence Synthetic oligonucleotide
gtctagttctgtcctctatttc

SEQ ID NO: 72 - DNA, 22 bp, Artificial Sequence
  Description of Artificial Sequence Synthetic oligonucleotide
agtctagttctgtcctctattt

SEQ ID NO: 73 - DNA, 22 bp, Artificial Sequence
  Description of Artificial Sequence Synthetic oligonucleotide
tatcagtctagttctgtcctct

SEQ ID NO: 74 - DNA, 22 bp, Artificial Sequence
  Description of Artificial Sequence Synthetic oligonucleotide
ctatcagtctagttctgtcctc

SEQ ID NO: 75 - DNA, 22 bp, Artificial Sequence
  Description of Artificial Sequence Synthetic oligonucleotide
tgctatcagtctagttctgtcc

SEQ ID NO: 76 - DNA, 22 bp, Artificial Sequence
  Description of Artificial Sequence Synthetic oligonucleotide
aattgttctaggtcactgctat

SEQ ID NO: 77 - DNA, 22 bp, Artificial Sequence
  Description of Artificial Sequence Synthetic oligonucleotide
agtgaatatgagacaagcttcc

SEQ ID NO: 78 - DNA, 22 bp, Artificial Sequence
  Description of Artificial Sequence Synthetic oligonucleotide
gagtgaatatgagacaagcttc

SEQ ID NO: 79 - DNA, 22 bp, Artificial Sequence
  Description of Artificial Sequence Synthetic oligonucleotide
cacattagattgttctgttccc

SEQ ID NO: 80 - DNA, 22 bp, Artificial Sequence
  Description of Artificial Sequence Synthetic oligonucleotide
atagctacatactggataagcc

SEQ ID NO: 81 - DNA, 22 bp, Artificial Sequence
  Description of Artificial Sequence Synthetic oligonucleotide
aatagctacatactggataagc

SEQ ID NO: 82 - DNA, 21 bp, Artificial Sequence
  Description of Artificial Sequence Synthetic oligonucleotide
acactgtcgtcgaatggccac

SEQ ID NO: 83 - DNA, 21 bp, Artificial Sequence
  Description of Artificial Sequence Synthetic oligonucleotide
gctaatgaattcctttacacc

SEQ ID NO: 84 - DNA, 21 bp, Artificial Sequence
  Description of Artificial Sequence Synthetic oligonucleotide
atacatccatggctaatgaat

SEQ ID NO: 85 - DNA, 21 bp, Artificial Sequence
  Description of Artificial Sequence Synthetic oligonucleotide
acaactccctccttggccttt

SEQ ID NO: 86 - DNA, 21 bp, Artificial Sequence
  Description of Artificial Sequence Synthetic oligonucleotide
gtctttcctgctgcttctgcc

SEQ ID NO: 87 - DNA, 21 bp, Artificial Sequence
  Description of Artificial Sequence Synthetic oligonucleotide
tgtctttcctgctgcttctgc

SEQ ID NO: 88 - DNA, 21 bp, Artificial Sequence
  Description of Artificial Sequence Synthetic oligonucleotide
cttctcagccactgttgccac

SEQ ID NO: 89 - DNA, 21 bp, Artificial Sequence
  Description of Artificial Sequence Synthetic oligonucleotide
gtcttctcagccactgttgcc

SEQ ID NO: 90 - DNA, 21 bp, Artificial Sequence
  Description of Artificial Sequence Synthetic oligonucleotide
ggtcttctcagccactgttgc

SEQ ID NO: 91 - DNA, 21 bp, Artificial Sequence
  Description of Artificial Sequence Synthetic oligonucleotide
ctctttggtcttctcagccac

SEQ ID NO: 92 - DNA, 21 bp, Artificial Sequence
  Description of Artificial Sequence Synthetic oligonucleotide
tcacttgctctttggtcttct

SEQ ID NO: 93 - DNA, 21 bp, Artificial Sequence
  Description of Artificial Sequence Synthetic oligonucleotide
gtcacttgctctttggtcttc

SEQ ID NO: 94 - DNA, 21 bp, Artificial Sequence
  Description of Artificial Sequence Synthetic oligonucleotide
ttgtcacttgctctttggtct

SEQ ID NO: 95 - DNA, 21 bp, Artificial Sequence
  Description of Artificial Sequence Synthetic oligonucleotide
tttgtcacttgctctttggtc

SEQ ID NO: 96 - DNA, 21 bp, Artificial Sequence
  Description of Artificial Sequence Synthetic oligonucleotide
aacatttgtcacttgctcttt

SEQ ID NO: 97 - DNA, 21 bp, Artificial Sequence
  Description of Artificial Sequence Synthetic oligonucleotide
gtcttctgggctactgctgtc

SEQ ID NO: 98 - DNA, 21 bp, Artificial Sequence
  Description of Artificial Sequence Synthetic oligonucleotide
tgtcttctgggctactgctgt

SEQ ID NO: 99 - DNA, 21 bp, Artificial Sequence
  Description of Artificial Sequence Synthetic oligonucleotide
actgtcttctgggctactgct

SEQ ID NO: 100 - DNA, 21 bp, Artificial Sequence
  Description of Artificial Sequence Synthetic oligonucleotide
ccagtggctgctgcaatgctc

SEQ ID NO: 101 - DNA, 21 bp, Artificial Sequence
  Description of Artificial Sequence Synthetic oligonucleotide
agaattccttcctgtggggct

SEQ ID NO: 102 - DNA, 21 bp, Artificial Sequence
  Description of Artificial Sequence Synthetic oligonucleotide
cagaattccttcctgtggggc

SEQ ID NO: 103 - DNA, 21 bp, Artificial Sequence
  Description of Artificial Sequence Synthetic oligonucleotide
tatcttccagaattccttcct

SEQ ID NO: 104 - DNA, 21 bp, Artificial Sequence
  Description of Artificial Sequence Synthetic oligonucleotide
atatcttccagaattccttcc

SEQ ID NO: 105 - DNA, 21 bp, Artificial Sequence
  Description of Artificial Sequence Synthetic oligonucleotide
catatcttccagaattccttc

SEQ ID NO: 106 - DNA, 21 bp, Artificial Sequence
  Description of Artificial Sequence Synthetic oligonucleotide
attgtcaggatccacaggcat

SEQ ID NO: 107 - DNA, 21 bp, Artificial Sequence
  Description of Artificial Sequence Synthetic oligonucleotide
tcattgtcaggatccacaggc

SEQ ID NO: 108 - DNA, 21 bp, Artificial Sequence
  Description of Artificial Sequence Synthetic oligonucleotide
tcagaaggcatttcataagcc

SEQ ID NO: 109 - DNA, 21 bp, Artificial Sequence
  Description of Artificial Sequence Synthetic oligonucleotide
ctcagaaggcatttcataagc

SEQ ID NO: 110 - DNA, 21 bp, Artificial Sequence
  Description of Artificial Sequence Synthetic oligonucleotide
gtagtcttgatacccttcctc

SEQ ID NO: 111 - DNA, 21 bp, Artificial Sequence
  Description of Artificial Sequence Synthetic oligonucleotide
tcgtagtcttgatacccttcc

SEQ ID NO: 112 - DNA, 21 bp, Artificial Sequence
  Description of Artificial Sequence Synthetic oligonucleotide
tttcttaggcttcaggttcgt

SEQ ID NO: 113 - DNA, 21 bp, Artificial Sequence
  Description of Artificial Sequence Synthetic oligonucleotide
tatttcttaggcttcaggttc

SEQ ID NO: 114 - DNA, 21 bp, Artificial Sequence
  Description of Artificial Sequence Synthetic oligonucleotide
atatttcttaggcttcaggtt

SEQ ID NO: 115 - DNA, 21 bp, Artificial Sequence
  Description of Artificial Sequence Synthetic oligonucleotide
agatctcaagaaactgggagc

SEQ ID NO: 116 - DNA, 21 bp, Artificial Sequence
  Description of Artificial Sequence Synthetic oligonucleotide
agcacttgtacaggatggaac

SEQ ID NO: 117 - DNA, 21 bp, Artificial Sequence
  Description of Artificial Sequence Synthetic oligonucleotide
aactgagcacttgtacaggat

SEQ ID NO: 118 - DNA, 21 bp, Artificial Sequence
  Description of Artificial Sequence Synthetic oligonucleotide
atgactgggcacattggaact

SEQ ID NO: 119 - DNA, 21 bp, Artificial Sequence
  Description of Artificial Sequence Synthetic oligonucleotide
catgactgggcacattggaac

SEQ ID NO: 120 - DNA, 21 bp, Artificial Sequence
  Description of Artificial Sequence Synthetic oligonucleotide
acagatacttcaatcactgct

SEQ ID NO: 121 - DNA, 21 bp, Artificial Sequence
  Description of Artificial Sequence Synthetic oligonucleotide
ttaacatcgtagattgaagcc

SEQ ID NO: 122 - DNA, 21 bp, Artificial Sequence
  Description of Artificial Sequence Synthetic oligonucleotide
tttaacatcgtagattgaagc

SEQ ID NO: 123 - DNA, 21 bp, Artificial Sequence
  Description of Artificial Sequence Synthetic oligonucleotide
ttagaaataagtggtagtcac

SEQ ID NO: 124 - DNA, 21 bp, Artificial Sequence
  Description of Artificial Sequence Synthetic oligonucleotide
tcagttcttaattcatgttgc

SEQ ID NO: 125 - DNA, 21 bp, Artificial Sequence
  Description of Artificial Sequence Synthetic oligonucleotide
agtgtagggttaatgttccat

SEQ ID NO: 126 - DNA, 21 bp, Artificial Sequence
  Description of Artificial Sequence Synthetic oligonucleotide
cagtgttgcttcagggaattc

SEQ ID NO: 127 - DNA, 21 bp, Artificial Sequence
  Description of Artificial Sequence Synthetic oligonucleotide
cttaaggaaccagtgcatacc

SEQ ID NO: 128 - DNA, 21 bp, Artificial Sequence
  Description of Artificial Sequence Synthetic oligonucleotide
atcacagccacttaaggaacc

SEQ ID NO: 129 - DNA, 21 bp, Artificial Sequence
  Description of Artificial Sequence Synthetic oligonucleotide
aatcacagccacttaaggaac

SEQ ID NO: 130 - DNA, 21 bp, Artificial Sequence
  Description of Artificial Sequence Synthetic oligonucleotide
tcaataattaatcacagccac

SEQ ID NO: 131 - DNA, 21 bp, Artificial Sequence
  Description of Artificial Sequence Synthetic oligonucleotide
tacaatagtagttggggtctt

SEQ ID NO: 132 - DNA, 21 bp, Artificial Sequence
  Description of Artificial Sequence Synthetic oligonucleotide
gattgaagggagaaatagacc

SEQ ID NO: 133 - DNA, 21 bp, Artificial Sequence
  Description of Artificial Sequence Synthetic oligonucleotide
tcagaaaggtacagcattcac

SEQ ID NO: 134 - DNA, 21 bp, Artificial Sequence
  Description of Artificial Sequence Synthetic oligonucleotide
tgtcagaaaggtacagcattc

SEQ ID NO: 135 - DNA, 21 bp, Artificial Sequence
  Description of Artificial Sequence Synthetic oligonucleotide
ttgtcagaaaggtacagcatt

SEQ ID NO: 136 - DNA, 21 bp, Artificial Sequence
  Description of Artificial Sequence Synthetic oligonucleotide
attgtcagaaaggtacagcat

SEQ ID NO: 137 - DNA, 21 bp, Artificial Sequence
  Description of Artificial Sequence Synthetic oligonucleotide
cttctacactgcttagttccc

SEQ ID NO: 138 - DNA, 21 bp, Artificial Sequence
  Description of Artificial Sequence Synthetic oligonucleotide
tcttctacactgcttagttcc

SEQ ID NO: 139 - DNA, 21 bp, Artificial Sequence
  Description of Artificial Sequence Synthetic oligonucleotide
aaatcatcttctacactgctt

SEQ ID NO: 140 - DNA, 21 bp, Artificial Sequence
  Description of Artificial Sequence Synthetic oligonucleotide
ttgaatgtgctaatatgtgct

SEQ ID NO: 141 - DNA, 21 bp, Artificial Sequence
  Description of Artificial Sequence Synthetic oligonucleotide
cttgaatgtgctaatatgtgc

SEQ ID NO: 142 - DNA, 21 bp, Artificial Sequence
  Description of Artificial Sequence Synthetic oligonucleotide
tctcagagccttgaatgtgct

SEQ ID NO: 143 - DNA, 21 bp, Artificial Sequence
  Description of Artificial Sequence Synthetic oligonucleotide
atgtttaaaggcatttcctgt

SEQ ID NO: 144 - DNA, 21 bp, Artificial Sequence
  Description of Artificial Sequence Synthetic oligonucleotide
ggtgactctggtagttccaac

SEQ ID NO: 145 - DNA, 21 bp, Artificial Sequence
  Description of Artificial Sequence Synthetic oligonucleotide
taaggtgactctggtagttcc

SEQ ID NO: 146 - DNA, 21 bp, Artificial Sequence
  Description of Artificial Sequence Synthetic oligonucleotide
ttaaggtgactctggtagttc

SEQ ID NO: 147 - DNA, 21 bp, Artificial Sequence
  Description of Artificial Sequence Synthetic oligonucleotide
tttaaaggaggccatgaaatt

SEQ ID NO: 148 - DNA, 21 bp, Artificial Sequence
  Description of Artificial Sequence Synthetic oligonucleotide
cctagaattcatatatttggc

SEQ ID NO: 149 - DNA, 21 bp, Artificial Sequence
  Description of Artificial Sequence Synthetic oligonucleotide
ctcattcctccttccttcctc

SEQ ID NO: 150 - DNA, 21 bp, Artificial Sequence
  Description of Artificial Sequence Synthetic oligonucleotide
actcattcctccttccttcct

SEQ ID NO: 151 - DNA, 21 bp, Artificial Sequence
  Description of Artificial Sequence Synthetic oligonucleotide
cactcattcctccttccttcc

SEQ ID NO: 152 - DNA, 21 bp, Artificial Sequence
  Description of Artificial Sequence Synthetic oligonucleotide
tcactcattcctccttccttc

SEQ ID NO: 153 - DNA, 21 bp, Artificial Sequence
  Description of Artificial Sequence Synthetic oligonucleotide
actctgtagtagtctctcttc

SEQ ID NO: 154 - DNA, 21 bp, Artificial Sequence
  Description of Artificial Sequence Synthetic oligonucleotide
atatcctaaccgccactttct

SEQ ID NO: 155 - DNA, 21 bp, Artificial Sequence
  Description of Artificial Sequence Synthetic oligonucleotide
atatatcctaaccgccacttt

SEQ ID NO: 156 - DNA, 21 bp, Artificial Sequence
  Description of Artificial Sequence Synthetic oligonucleotide
aatatatcctaaccgccactt

SEQ ID NO: 157 - DNA, 21 bp, Artificial Sequence
  Description of Artificial Sequence Synthetic oligonucleotide
aaatatatcctaaccgccact

SEQ ID NO: 158 - DNA, 21 bp, Artificial Sequence
  Description of Artificial Sequence Synthetic oligonucleotide
tagttctgtcctctatttctt

SEQ ID NO: 159 - DNA, 21 bp, Artificial Sequence
  Description of Artificial Sequence Synthetic oligonucleotide
tctagttctgtcctctatttc

SEQ ID NO: 160 - DNA, 21 bp, Artificial Sequence
  Description of Artificial Sequence Synthetic oligonucleotide
tatcagtctagttctgtcctc

SEQ ID NO: 161 - DNA, 21 bp, Artificial Sequence
  Description of Artificial Sequence Synthetic oligonucleotide
gctatcagtctagttctgtcc

SEQ ID NO: 162 - DNA, 21 bp, Artificial Sequence
  Description of Artificial Sequence Synthetic oligonucleotide
attgttctaggtcactgctat

SEQ ID NO: 163 - DNA, 21 bp, Artificial Sequence
  Description of Artificial Sequence Synthetic oligonucleotide
aaattgttctaggtcactgct

SEQ ID NO: 164 - DNA, 21 bp, Artificial Sequence
  Description of Artificial Sequence Synthetic oligonucleotide
tttgtttaagtgtttggtccc

SEQ ID NO: 165 - DNA, 21 bp, Artificial Sequence
  Description of Artificial Sequence Synthetic oligonucleotide
tgaatatgagacaagcttcct

SEQ ID NO: 166 - DNA, 21 bp, Artificial Sequence
  Description of Artificial Sequence Synthetic oligonucleotide
gtgaatatgagacaagcttcc

SEQ ID NO: 167 - DNA, 21 bp, Artificial Sequence
  Description of Artificial Sequence Synthetic oligonucleotide
agtgaatatgagacaagcttc

SEQ ID NO: 168 - DNA, 21 bp, Artificial Sequence
  Description of Artificial Sequence Synthetic oligonucleotide
aaataaacctaggactggatt

SEQ ID NO: 169 - DNA, 21 bp, Artificial Sequence
  Description of Artificial Sequence Synthetic oligonucleotide
tggtaaagccgaccgtggagt

SEQ ID NO: 170 - DNA, 21 bp, Artificial Sequence
  Description of Artificial Sequence Synthetic oligonucleotide
acattagattgttctgttccc

SEQ ID NO: 171 - DNA, 21 bp, Artificial Sequence
  Description of Artificial Sequence Synthetic oligonucleotide
cacattagattgttctgttcc

SEQ ID NO: 172 - DNA, 21 bp, Artificial Sequence
  Description of Artificial Sequence Synthetic oligonucleotide
tagctacatactggataagcc

SEQ ID NO: 173 - DNA, 21 bp, Artificial Sequence
  Description of Artificial Sequence Synthetic oligonucleotide
atagctacatactggataagc

SEQ ID NO: 174 - DNA, 20 bp, Artificial Sequence
  Description of Artificial Sequence Synthetic oligonucleotide
agttctccgctcacgagggt

SEQ ID NO: 175 - DNA, 20 bp, Artificial Sequence
  Description of Artificial Sequence Synthetic oligonucleotide
ccactcccagttctccgctc

SEQ ID NO: 176 - DNA, 20 bp, Artificial Sequence
  Description of Artificial Sequence Synthetic oligonucleotide
cactgtcgtcgaatggccac

SEQ ID NO: 177 - DNA, 20 bp, Artificial Sequence
  Description of Artificial Sequence Synthetic oligonucleotide
cacactgtcgtcgaatggcc

SEQ ID NO: 178 - DNA, 20 bp, Artificial Sequence
  Description of Artificial Sequence Synthetic oligonucleotide
ctaatgaattcctttacacc

SEQ ID NO: 179 - DNA, 20 bp, Artificial Sequence
  Description of Artificial Sequence Synthetic oligonucleotide
acaactccctccttggcctt

SEQ ID NO: 180 - DNA, 20 bp, Artificial Sequence
  Description of Artificial Sequence Synthetic oligonucleotide
gtctttcctgctgcttctgc

SEQ ID NO: 181 - DNA, 20 bp, Artificial Sequence
  Description of Artificial Sequence Synthetic oligonucleotide
ttgtctttcctgctgcttct

SEQ ID NO: 182 - DNA, 20 bp, Artificial Sequence
  Description of Artificial Sequence Synthetic oligonucleotide
tttgtctttcctgctgcttc

SEQ ID NO: 183 - DNA, 20 bp, Artificial Sequence
  Description of Artificial Sequence Synthetic oligonucleotide
ttctcagccactgttgccac

SEQ ID NO: 184 - DNA, 20 bp, Artificial Sequence
  Description of Artificial Sequence Synthetic oligonucleotide
tcttctcagccactgttgcc

SEQ ID NO: 185 - DNA, 20 bp, Artificial Sequence
  Description of Artificial Sequence Synthetic oligonucleotide
gtcttctcagccactgttgc

SEQ ID NO: 186 - DNA, 20 bp, Artificial Sequence
  Description of Artificial Sequence Synthetic oligonucleotide
gctctttggtcttctcagcc

SEQ ID NO: 187 - DNA, 20 bp, Artificial Sequence
  Description of Artificial Sequence Synthetic oligonucleotide
tcacttgctctttggtcttc

SEQ ID NO: 188 - DNA, 20 bp, Artificial Sequence
  Description of Artificial Sequence Synthetic oligonucleotide
tgtcacttgctctttggtct

SEQ ID NO: 189 - DNA, 20 bp, Artificial Sequence
  Description of Artificial Sequence Synthetic oligonucleotide
ttgtcacttgctctttggtc

SEQ ID NO: 190 - DNA, 20 bp, Artificial Sequence
  Description of Artificial Sequence Synthetic oligonucleotide
tttgtcacttgctctttggt

SEQ ID NO: 191 - DNA, 20 bp, Artificial Sequence
  Description of Artificial Sequence Synthetic oligonucleotide
acatttgtcacttgctcttt

SEQ ID NO: 192 - DNA, 20 bp, Artificial Sequence
  Description of Artificial Sequence Synthetic oligonucleotide
aacatttgtcacttgctctt

SEQ ID NO: 193 - DNA, 20 bp, Artificial Sequence
  Description of Artificial Sequence Synthetic oligonucleotide
actgtcttctgggctactgc

SEQ ID NO: 194 - DNA, 20 bp, Artificial Sequence
  Description of Artificial Sequence Synthetic oligonucleotide
agaattccttcctgtggggc

SEQ ID NO: 195 - DNA, 20 bp, Artificial Sequence
  Description of Artificial Sequence Synthetic oligonucleotide
tatcttccagaattccttcc

SEQ ID NO: 196 - DNA, 20 bp, Artificial Sequence
  Description of Artificial Sequence Synthetic oligonucleotide
atatcttccagaattccttc

SEQ ID NO: 197 - DNA, 20 bp, Artificial Sequence
  Description of Artificial Sequence Synthetic oligonucleotide
ttgtcaggatccacaggcat

SEQ ID NO: 198 - DNA, 20 bp, Artificial Sequence
  Description of Artificial Sequence Synthetic oligonucleotide
cattgtcaggatccacaggc

SEQ ID NO: 199 - DNA, 20 bp, Artificial Sequence
  Description of Artificial Sequence Synthetic oligonucleotide
tcagaaggcatttcataagc

SEQ ID NO: 200 - DNA, 20 bp, Artificial Sequence
  Description of Artificial Sequence Synthetic oligonucleotide
tagtcttgatacccttcctc

SEQ ID NO: 201 - DNA, 20 bp, Artificial Sequence
  Description of Artificial Sequence Synthetic oligonucleotide
cgtagtcttgatacccttcc

SEQ ID NO: 202 - DNA, 20 bp, Artificial Sequence
  Description of Artificial Sequence Synthetic oligonucleotide
tcgtagtcttgatacccttc

SEQ ID NO: 203 - DNA, 20 bp, Artificial Sequence
  Description of Artificial Sequence Synthetic oligonucleotide
ttcttaggcttcaggttcgt

SEQ ID NO: 204 - DNA, 20 bp, Artificial Sequence
  Description of Artificial Sequence Synthetic oligonucleotide
atttcttaggcttcaggttc

SEQ ID NO: 205 - DNA, 20 bp, Artificial Sequence
  Description of Artificial Sequence Synthetic oligonucleotide
tatttcttaggcttcaggtt

SEQ ID NO: 206 - DNA, 20 bp, Artificial Sequence
  Description of Artificial Sequence Synthetic oligonucleotide
atatttcttaggcttcaggt

SEQ ID NO: 207 - DNA, 20 bp, Artificial Sequence
  Description of Artificial Sequence Synthetic oligonucleotide
gatctcaagaaactgggagc

SEQ ID NO: 208 - DNA, 20 bp, Artificial Sequence
  Description of Artificial Sequence Synthetic oligonucleotide
tgactgggcacattggaact

SEQ ID NO: 209 - DNA, 20 bp, Artificial Sequence
  Description of Artificial Sequence Synthetic oligonucleotide
atgactgggcacattggaac

SEQ ID NO: 210 - DNA, 20 bp, Artificial Sequence
  Description of Artificial Sequence Synthetic oligonucleotide
tcactgctgatggaagactt

SEQ ID NO: 211 - DNA, 20 bp, Artificial Sequence
  Description of Artificial Sequence Synthetic oligonucleotide
acagatacttcaatcactgc

SEQ ID NO: 212 - DNA, 20 bp, Artificial Sequence
  Description of Artificial Sequence Synthetic oligonucleotide
taacatcgtagattgaagcc

SEQ ID NO: 213 - DNA, 20 bp, Artificial Sequence
  Description of Artificial Sequence Synthetic oligonucleotide
ttaacatcgtagattgaagc

SEQ ID NO: 214 - DNA, 20 bp, Artificial Sequence
  Description of Artificial Sequence Synthetic oligonucleotide
tagaaataagtggtagtcac

SEQ ID NO: 215 - DNA, 20 bp, Artificial Sequence
  Description of Artificial Sequence Synthetic oligonucleotide
tagtttcatgctcacatatt

SEQ ID NO: 216 - DNA, 20 bp, Artificial Sequence
  Description of Artificial Sequence Synthetic oligonucleotide
atagtttcatgctcacatat

SEQ ID NO: 217 - DNA, 20 bp, Artificial Sequence
  Description of Artificial Sequence Synthetic oligonucleotide
ttataggtgcatagtttcat

SEQ ID NO: 218 - DNA, 20 bp, Artificial Sequence
  Description of Artificial Sequence Synthetic oligonucleotide
atttataggtgcatagtttc

SEQ ID NO: 219 - DNA, 20 bp, Artificial Sequence
  Description of Artificial Sequence Synthetic oligonucleotide
agtatttataggtgcatagt

SEQ ID NO: 220 - DNA, 20 bp, Artificial Sequence
  Description of Artificial Sequence Synthetic oligonucleotide
ttattaaagtgagatgggat

SEQ ID NO: 221 - DNA, 20 bp, Artificial Sequence
  Description of Artificial Sequence Synthetic oligonucleotide
agttcttaattcatgttgct

SEQ ID NO: 222 - DNA, 20 bp, Artificial Sequence
  Description of Artificial Sequence Synthetic oligonucleotide
cagttcttaattcatgttgc

SEQ ID NO: 223 - DNA, 20 bp, Artificial Sequence
  Description of Artificial Sequence Synthetic oligonucleotide
gagtgtagggttaatgttcc

SEQ ID NO: 224 - DNA, 20 bp, Artificial Sequence
  Description of Artificial Sequence Synthetic oligonucleotide
agtgttgcttcagggaattc

SEQ ID NO: 225 - DNA, 20 bp, Artificial Sequence
  Description of Artificial Sequence Synthetic oligonucleotide
acttctggcagtgttgcttc

SEQ ID NO: 226 - DNA, 20 bp, Artificial Sequence
  Description of Artificial Sequence Synthetic oligonucleotide
tcacagccacttaaggaacc

SEQ ID NO: 227 - DNA, 20 bp, Artificial Sequence
  Description of Artificial Sequence Synthetic oligonucleotide
atcacagccacttaaggaac

SEQ ID NO: 228 - DNA, 20 bp, Artificial Sequence
  Description of Artificial Sequence Synthetic oligonucleotide
ataattaatcacagccactt

SEQ ID NO: 229 - DNA, 20 bp, Artificial Sequence
  Description of Artificial Sequence Synthetic oligonucleotide
aataattaatcacagccact

SEQ ID NO: 230 - DNA, 20 bp, Artificial Sequence
  Description of Artificial Sequence Synthetic oligonucleotide
caataattaatcacagccac

SEQ ID NO: 231 - DNA, 20 bp, Artificial Sequence
  Description of Artificial Sequence Synthetic oligonucleotide
ttcaataattaatcacagcc

SEQ ID NO: 232 - DNA, 20 bp, Artificial Sequence
  Description of Artificial Sequence Synthetic oligonucleotide
tacaatagtagttggggtct

SEQ ID NO: 233 - DNA, 20 bp, Artificial Sequence
  Description of Artificial Sequence Synthetic oligonucleotide
attgaagggagaaatagacc

SEQ ID NO: 234 - DNA, 20 bp, Artificial Sequence
  Description of Artificial Sequence Synthetic oligonucleotide
gtcagaaaggtacagcattc

SEQ ID NO: 235 - DNA, 20 bp, Artificial Sequence
  Description of Artificial Sequence Synthetic oligonucleotide
tgtcagaaaggtacagcatt

SEQ ID NO: 236 - DNA, 20 bp, Artificial Sequence
  Description of Artificial Sequence Synthetic oligonucleotide
ttgtcagaaaggtacagcat

SEQ ID NO: 237 - DNA, 20 bp, Artificial Sequence
  Description of Artificial Sequence Synthetic oligonucleotide
tattgtcagaaaggtacagc

SEQ ID NO: 238 - DNA, 20 bp, Artificial Sequence
  Description of Artificial Sequence Synthetic oligonucleotide
atttattgtcagaaaggtac

SEQ ID NO: 239 - DNA, 20 bp, Artificial Sequence
  Description of Artificial Sequence Synthetic oligonucleotide
ttctacactgcttagttccc

SEQ ID NO: 240 - DNA, 20 bp, Artificial Sequence
  Description of Artificial Sequence Synthetic oligonucleotide
cttctacactgcttagttcc

SEQ ID NO: 241 - DNA, 20 bp, Artificial Sequence
  Description of Artificial Sequence Synthetic oligonucleotide
aatcatcttctacactgctt

SEQ ID NO: 242 - DNA, 20 bp, Artificial Sequence
  Description of Artificial Sequence Synthetic oligonucleotide
aaatcatcttctacactgct

SEQ ID NO: 243 - DNA, 20 bp, Artificial Sequence
  Description of Artificial Sequence Synthetic oligonucleotide
tgaatgtgctaatatgtgct

SEQ ID NO: 244 - DNA, 20 bp, Artificial Sequence
  Description of Artificial Sequence Synthetic oligonucleotide
ttgaatgtgctaatatgtgc

SEQ ID NO: 245 - DNA, 20 bp, Artificial Sequence
  Description of Artificial Sequence Synthetic oligonucleotide
tctcagagccttgaatgtgc

SEQ ID NO: 246 - DNA, 20 bp, Artificial Sequence
  Description of Artificial Sequence Synthetic oligonucleotide
tgtttaaaggcatttcctgt

SEQ ID NO: 247 - DNA, 20 bp, Artificial Sequence
  Description of Artificial Sequence Synthetic oligonucleotide
gtgactctggtagttccaac

SEQ ID NO: 248 - DNA, 20 bp, Artificial Sequence
  Description of Artificial Sequence Synthetic oligonucleotide
aaggtgactctggtagttcc

SEQ ID NO: 249 - DNA, 20 bp, Artificial Sequence
  Description of Artificial Sequence Synthetic oligonucleotide
tttaaaggaggccatgaaat

SEQ ID NO: 250 - DNA, 20 bp, Artificial Sequence
  Description of Artificial Sequence Synthetic oligonucleotide
gaattcatatatttggcaac

SEQ ID NO: 251 - DNA, 20 bp, Artificial Sequence
  Description of Artificial Sequence Synthetic oligonucleotide
ctagaattcatatatttggc

SEQ ID NO: 252 - DNA, 20 bp, Artificial Sequence
  Description of Artificial Sequence Synthetic oligonucleotide
tactttctactagtgacttt

SEQ ID NO: 253 - DNA, 20 bp, Artificial Sequence
  Description of Artificial Sequence Synthetic oligonucleotide
atactttctactagtgactt

SEQ ID NO: 254 - DNA, 20 bp, Artificial Sequence
  Description of Artificial Sequence Synthetic oligonucleotide
tatactttctactagtgact

SEQ ID NO: 255 - DNA, 20 bp, Artificial Sequence
  Description of Artificial Sequence Synthetic oligonucleotide
ttatactttctactagtgac

SEQ ID NO: 256 - DNA, 20 bp, Artificial Sequence
  Description of Artificial Sequence Synthetic oligonucleotide
aatacatataaactgctagc

SEQ ID NO: 257 - DNA, 20 bp, Artificial Sequence
  Description of Artificial Sequence Synthetic oligonucleotide
catgaatacatataaactgc

SEQ ID NO: 258 - DNA, 20 bp, Artificial Sequence
  Description of Artificial Sequence Synthetic oligonucleotide
actcattcctccttccttcc

SEQ ID NO: 259 - DNA, 20 bp, Artificial Sequence
  Description of Artificial Sequence Synthetic oligonucleotide
cactcattcctccttccttc

SEQ ID NO: 260 - DNA, 20 bp, Artificial Sequence
  Description of Artificial Sequence Synthetic oligonucleotide
tcactcattcctccttcctt

SEQ ID NO: 261 - DNA, 20 bp, Artificial Sequence
  Description of Artificial Sequence Synthetic oligonucleotide
tagtctctcttcaattaggt

SEQ ID NO: 262 - DNA, 20 bp, Artificial Sequence
  Description of Artificial Sequence Synthetic oligonucleotide
actctgtagtagtctctctt

SEQ ID NO: 263 - DNA, 20 bp, Artificial Sequence
  Description of Artificial Sequence Synthetic oligonucleotide
atatcctaaccgccactttc

SEQ ID NO: 264 - DNA, 20 bp, Artificial Sequence
  Description of Artificial Sequence Synthetic oligonucleotide
atatatcctaaccgccactt

SEQ ID NO: 265 - DNA, 20 bp, Artificial Sequence
  Description of Artificial Sequence Synthetic oligonucleotide
aatatatcctaaccgccact

SEQ ID NO: 266 - DNA, 20 bp, Artificial Sequence
  Description of Artificial Sequence Synthetic oligonucleotide
aaatatatcctaaccgccac

SEQ ID NO: 267 - DNA, 20 bp, Artificial Sequence
  Description of Artificial Sequence Synthetic oligonucleotide
agttctgtcctctatttctt

SEQ ID NO: 268 - DNA, 20 bp, Artificial Sequence
  Description of Artificial Sequence Synthetic oligonucleotide
tagttctgtcctctatttct

SEQ ID NO: 269 - DNA, 20 bp, Artificial Sequence
  Description of Artificial Sequence Synthetic oligonucleotide
ctagttctgtcctctatttc

SEQ ID NO: 270 - DNA, 20 bp, Artificial Sequence
  Description of Artificial Sequence Synthetic oligonucleotide
tctagttctgtcctctattt

SEQ ID NO: 271 - DNA, 20 bp, Artificial Sequence
  Description of Artificial Sequence Synthetic oligonucleotide
tcagtctagttctgtcctct

SEQ ID NO: 272 - DNA, 20 bp, Artificial Sequence
  Description of Artificial Sequence Synthetic oligonucleotide
tatcagtctagttctgtcct

SEQ ID NO: 273 - DNA, 20 bp, Artificial Sequence
  Description of Artificial Sequence Synthetic oligonucleotide
ctatcagtctagttctgtcc

SEQ ID NO: 274 - DNA, 20 bp, Artificial Sequence
  Description of Artificial Sequence Synthetic oligonucleotide
ttgttctaggtcactgctat

SEQ ID NO: 275 - DNA, 20 bp, Artificial Sequence
  Description of Artificial Sequence Synthetic oligonucleotide
aattgttctaggtcactgct

SEQ ID NO: 276 - DNA, 20 bp, Artificial Sequence
  Description of Artificial Sequence Synthetic oligonucleotide
aaattgttctaggtcactgc

SEQ ID NO: 277 - DNA, 20 bp, Artificial Sequence
  Description of Artificial Sequence Synthetic oligonucleotide
ttgtatccacataaatcctt

SEQ ID NO: 278 - DNA, 20 bp, Artificial Sequence
  Description of Artificial Sequence Synthetic oligonucleotide
tttgtatccacataaatcct

SEQ ID NO: 279 - DNA, 20 bp, Artificial Sequence
  Description of Artificial Sequence Synthetic oligonucleotide
tataaagtaattcactgctc

SEQ ID NO: 280 - DNA, 20 bp, Artificial Sequence
  Description of Artificial Sequence Synthetic oligonucleotide
ttgtttaagtgtttggtccc

SEQ ID NO: 281 - DNA, 20 bp, Artificial Sequence
  Description of Artificial Sequence Synthetic oligonucleotide
tttgtttaagtgtttggtcc

SEQ ID NO: 282 - DNA, 20 bp, Artificial Sequence
  Description of Artificial Sequence Synthetic oligonucleotide
tgaatatgagacaagcttcc

SEQ ID NO: 283 - DNA, 20 bp, Artificial Sequence
  Description of Artificial Sequence Synthetic oligonucleotide
gtgaatatgagacaagcttc

SEQ ID NO: 284 - DNA, 20 bp, Artificial Sequence
  Description of Artificial Sequence Synthetic oligonucleotide
agtgaatatgagacaagctt

SEQ ID NO: 285 - DNA, 20 bp, Artificial Sequence
  Description of Artificial Sequence Synthetic oligonucleotide
ggagtgaatatgagacaagc

SEQ ID NO: 286 - DNA, 20 bp, Artificial Sequence
  Description of Artificial Sequence Synthetic oligonucleotide
tgaatgtctcgggagtgaat

SEQ ID NO: 287 - DNA, 20 bp, Artificial Sequence
  Description of Artificial Sequence Synthetic oligonucleotide
aataaacctaggactggatt

SEQ ID NO: 288 - DNA, 20 bp, Artificial Sequence
  Description of Artificial Sequence Synthetic oligonucleotide
aaataaacctaggactggat

SEQ ID NO: 289 - DNA, 20 bp, Artificial Sequence
  Description of Artificial Sequence Synthetic oligonucleotide
cattagattgttctgttccc

SEQ ID NO: 290 - DNA, 20 bp, Artificial Sequence
  Description of Artificial Sequence Synthetic oligonucleotide
acattagattgttctgttcc

SEQ ID NO: 291 - DNA, 20 bp, Artificial Sequence
  Description of Artificial Sequence Synthetic oligonucleotide
cacattagattgttctgttc

SEQ ID NO: 292 - DNA, 20 bp, Artificial Sequence
  Description of Artificial Sequence Synthetic oligonucleotide
tagctacatactggataagc

SEQ ID NO: 293 - DNA, 19 bp, Artificial Sequence
  Description of Artificial Sequence Synthetic oligonucleotide
acactgtcgtcgaatggcc

SEQ ID NO: 294 - DNA, 19 bp, Artificial Sequence
  Description of Artificial Sequence Synthetic oligonucleotide
cacactgtcgtcgaatggc

SEQ ID NO: 295 - DNA, 19 bp, Artificial Sequence
  Description of Artificial Sequence Synthetic oligonucleotide
taatgaattcctttacacc

SEQ ID NO: 296 - DNA, 19 bp, Artificial Sequence
  Description of Artificial Sequence Synthetic oligonucleotide
acaactccctccttggcct

SEQ ID NO: 297 - DNA, 19 bp, Artificial Sequence
  Description of Artificial Sequence Synthetic oligonucleotide
tgtctttcctgctgcttct

SEQ ID NO: 298 - DNA, 19 bp, Artificial Sequence
  Description of Artificial Sequence Synthetic oligonucleotide
ttgtctttcctgctgcttc

SEQ ID NO: 299 - DNA, 19 bp, Artificial Sequence
  Description of Artificial Sequence Synthetic oligonucleotide
tttgtctttcctgctgctt

SEQ ID NO: 300 - DNA, 19 bp, Artificial Sequence
  Description of Artificial Sequence Synthetic oligonucleotide
cttctcagccactgttgcc

SEQ ID NO: 301 - DNA, 19 bp, Artificial Sequence
  Description of Artificial Sequence Synthetic oligonucleotide
tcttctcagccactgttgc

SEQ ID NO: 302 - DNA, 19 bp, Artificial Sequence
  Description of Artificial Sequence Synthetic oligonucleotide
ctctttggtcttctcagcc

SEQ ID NO: 303 - DNA, 19 bp, Artificial Sequence
  Description of Artificial Sequence Synthetic oligonucleotide
gctctttggtcttctcagc

SEQ ID NO: 304 - DNA, 19 bp, Artificial Sequence
  Description of Artificial Sequence Synthetic oligonucleotide
tgtcacttgctctttggtc

SEQ ID NO: 305 - DNA, 19 bp, Artificial Sequence
  Description of Artificial Sequence Synthetic oligonucleotide
ttgtcacttgctctttggt

SEQ ID NO: 306 - DNA, 19 bp, Artificial Sequence
  Description of Artificial Sequence Synthetic oligonucleotide
acatttgtcacttgctctt

SEQ ID NO: 307 - DNA, 19 bp, Artificial Sequence
  Description of Artificial Sequence Synthetic oligonucleotide
aacatttgtcacttgctct

SEQ ID NO: 308 - DNA, 19 bp, Artificial Sequence
  Description of Artificial Sequence Synthetic oligonucleotide
caacatttgtcacttgctc

SEQ ID NO: 309 - DNA, 19 bp, Artificial Sequence
  Description of Artificial Sequence Synthetic oligonucleotide
tcttctgggctactgctgt

SEQ ID NO: 310 - DNA, 19 bp, Artificial Sequence
  Description of Artificial Sequence Synthetic oligonucleotide
tgtcttctgggctactgct

SEQ ID NO: 311 - DNA, 19 bp, Artificial Sequence
  Description of Artificial Sequence Synthetic oligonucleotide
ctgtcttctgggctactgc

SEQ ID NO: 312 - DNA, 19 bp, Artificial Sequence
  Description of Artificial Sequence Synthetic oligonucleotide
ccagtggctgctgcaatgc

SEQ ID NO: 313 - DNA, 19 bp, Artificial Sequence
  Description of Artificial Sequence Synthetic oligonucleotide
tatcttccagaattccttc

SEQ ID NO: 314 - DNA, 19 bp, Artificial Sequence
  Description of Artificial Sequence Synthetic oligonucleotide
atatcttccagaattcctt

SEQ ID NO: 315 - DNA, 19 bp, Artificial Sequence
  Description of Artificial Sequence Synthetic oligonucleotide
gcatatcttccagaattcc

SEQ ID NO: 316 - DNA, 19 bp, Artificial Sequence
  Description of Artificial Sequence Synthetic oligonucleotide
attgtcaggatccacaggc

SEQ ID NO: 317 - DNA, 19 bp, Artificial Sequence
  Description of Artificial Sequence Synthetic oligonucleotide
tagtcttgatacccttcct

SEQ ID NO: 318 - DNA, 19 bp, Artificial Sequence
  Description of Artificial Sequence Synthetic oligonucleotide
gtagtcttgatacccttcc

SEQ ID NO: 319 - DNA, 19 bp, Artificial Sequence
  Description of Artificial Sequence Synthetic oligonucleotide
cgtagtcttgatacccttc

SEQ ID NO: 320 - DNA, 19 bp, Artificial Sequence
  Description of Artificial Sequence Synthetic oligonucleotide
tcgtagtcttgataccctt

SEQ ID NO: 321 - DNA, 19 bp, Artificial Sequence
  Description of Artificial Sequence Synthetic oligonucleotide
tcttaggcttcaggttcgt

SEQ ID NO: 322 - DNA, 19 bp, Artificial Sequence
  Description of Artificial Sequence Synthetic oligonucleotide
tttcttaggcttcaggttc

SEQ ID NO: 323 - DNA, 19 bp, Artificial Sequence
  Description of Artificial Sequence Synthetic oligonucleotide
atttcttaggcttcaggtt

SEQ ID NO: 324 - DNA, 19 bp, Artificial Sequence
  Description of Artificial Sequence Synthetic oligonucleotide
tatttcttaggcttcaggt

SEQ ID NO: 325 - DNA, 19 bp, Artificial Sequence
  Description of Artificial Sequence Synthetic oligonucleotide
atctcaagaaactgggagc

SEQ ID NO: 326 - DNA, 19 bp, Artificial Sequence
  Description of Artificial Sequence Synthetic oligonucleotide
cacttgtacaggatggaac

SEQ ID NO: 327 - DNA, 19 bp, Artificial Sequence
  Description of Artificial Sequence Synthetic oligonucleotide
tgactgggcacattggaac

SEQ ID NO: 328 - DNA, 19 bp, Artificial Sequence
  Description of Artificial Sequence Synthetic oligonucleotide
agacttcgagatacactgt

SEQ ID NO: 329 - DNA, 19 bp, Artificial Sequence
  Description of Artificial Sequence Synthetic oligonucleotide
agatacttcaatcactgct

SEQ ID NO: 330 - DNA, 19 bp, Artificial Sequence
  Description of Artificial Sequence Synthetic oligonucleotide
cagatacttcaatcactgc

SEQ ID NO: 331 - DNA, 19 bp, Artificial Sequence
  Description of Artificial Sequence Synthetic oligonucleotide
gtacagatacttcaatcac

SEQ ID NO: 332 - DNA, 19 bp, Artificial Sequence
  Description of Artificial Sequence Synthetic oligonucleotide
tcagtgaaagggaagcacc

SEQ ID NO: 333 - DNA, 19 bp, Artificial Sequence
  Description of Artificial Sequence Synthetic oligonucleotide
taacatcgtagattgaagc

SEQ ID NO: 334 - DNA, 19 bp, Artificial Sequence
  Description of Artificial Sequence Synthetic oligonucleotide
tagtttcatgctcacatat

SEQ ID NO: 335 - DNA, 19 bp, Artificial Sequence
  Description of Artificial Sequence Synthetic oligonucleotide
tataggtgcatagtttcat

SEQ ID NO: 336 - DNA, 19 bp, Artificial Sequence
  Description of Artificial Sequence Synthetic oligonucleotide
tttataggtgcatagtttc

SEQ ID NO: 337 - DNA, 19 bp, Artificial Sequence
  Description of Artificial Sequence Synthetic oligonucleotide
tattaaagtgagatgggat

SEQ ID NO: 338 - DNA, 19 bp, Artificial Sequence
  Description of Artificial Sequence Synthetic oligonucleotide
agttcttaattcatgttgc

SEQ ID NO: 339 - DNA, 19 bp, Artificial Sequence
  Description of Artificial Sequence Synthetic oligonucleotide
agtgtagggttaatgttcc

SEQ ID NO: 340 - DNA, 19 bp, Artificial Sequence
  Description of Artificial Sequence Synthetic oligonucleotide
gagtgtagggttaatgttc

SEQ ID NO: 341 - DNA, 19 bp, Artificial Sequence
  Description of Artificial Sequence Synthetic oligonucleotide
agtgttgcttcagggaatt

SEQ ID NO: 342 - DNA, 19 bp, Artificial Sequence
  Description of Artificial Sequence Synthetic oligonucleotide
acttctggcagtgttgctt

SEQ ID NO: 343 - DNA, 19 bp, Artificial Sequence
  Description of Artificial Sequence Synthetic oligonucleotide
acttaaggaaccagtgcat

SEQ ID NO: 344 - DNA, 19 bp, Artificial Sequence
  Description of Artificial Sequence Synthetic oligonucleotide
tcacagccacttaaggaac

SEQ ID NO: 345 - DNA, 19 bp, Artificial Sequence
  Description of Artificial Sequence Synthetic oligonucleotide
taattaatcacagccactt

SEQ ID NO: 346 - DNA, 19 bp, Artificial Sequence
  Description of Artificial Sequence Synthetic oligonucleotide
ataattaatcacagccact

SEQ ID NO: 347 - DNA, 19 bp, Artificial Sequence
  Description of Artificial Sequence Synthetic oligonucleotide
aataattaatcacagccac

SEQ ID NO: 348 - DNA, 19 bp, Artificial Sequence
  Description of Artificial Sequence Synthetic oligonucleotide
tcaataattaatcacagcc

SEQ ID NO: 349 - DNA, 19 bp, Artificial Sequence
  Description of Artificial Sequence Synthetic oligonucleotide
aatagtagttggggtcttc

SEQ ID NO: 350 - DNA, 19 bp, Artificial Sequence
  Description of Artificial Sequence Synthetic oligonucleotide
tacaatagtagttggggtc

SEQ ID NO: 351 - DNA, 19 bp, Artificial Sequence
  Description of Artificial Sequence Synthetic oligonucleotide
tcagaaaggtacagcattc

SEQ ID NO: 352 - DNA, 19 bp, Artificial Sequence
  Description of Artificial Sequence Synthetic oligonucleotide
tgtcagaaaggtacagcat

SEQ ID NO: 353 - DNA, 19 bp, Artificial Sequence
  Description of Artificial Sequence Synthetic oligonucleotide
attgtcagaaaggtacagc

SEQ ID NO: 354 - DNA, 19 bp, Artificial Sequence
  Description of Artificial Sequence Synthetic oligonucleotide
tctacactgcttagttccc

SEQ ID NO: 355 - DNA, 19 bp, Artificial Sequence
  Description of Artificial Sequence Synthetic oligonucleotide
ttctacactgcttagttcc

SEQ ID NO: 356 - DNA, 19 bp, Artificial Sequence
  Description of Artificial Sequence Synthetic oligonucleotide
atcatcttctacactgctt

SEQ ID NO: 357 - DNA, 19 bp, Artificial Sequence
  Description of Artificial Sequence Synthetic oligonucleotide
aatcatcttctacactgct

SEQ ID NO: 358 - DNA, 19 bp, Artificial Sequence
  Description of Artificial Sequence Synthetic oligonucleotide
aaatcatcttctacactgc

SEQ ID NO: 359 - DNA, 19 bp, Artificial Sequence
  Description of Artificial Sequence Synthetic oligonucleotide
tgaatgtgctaatatgtgc

SEQ ID NO: 360 - DNA, 19 bp, Artificial Sequence
  Description of Artificial Sequence Synthetic oligonucleotide
tcagagccttgaatgtgct

SEQ ID NO: 361 - DNA, 19 bp, Artificial Sequence
  Description of Artificial Sequence Synthetic oligonucleotide
ctcagagccttgaatgtgc

SEQ ID NO: 362 - DNA, 19 bp, Artificial Sequence
  Description of Artificial Sequence Synthetic oligonucleotide
atgtttaaaggcatttcct

SEQ ID NO: 363 - DNA, 19 bp, Artificial Sequence
  Description of Artificial Sequence Synthetic oligonucleotide
gatgtttaaaggcatttcc

SEQ ID NO: 364 - DNA, 19 bp, Artificial Sequence
  Description of Artificial Sequence Synthetic oligonucleotide
tgactctggtagttccaac

SEQ ID NO: 365 - DNA, 19 bp, Artificial Sequence
  Description of Artificial Sequence Synthetic oligonucleotide
aacatttaaaggaggccat

SEQ ID NO: 366 - DNA, 19 bp, Artificial Sequence
  Description of Artificial Sequence Synthetic oligonucleotide
ggagttaatagatcttccc

SEQ ID NO: 367 - DNA, 19 bp, Artificial Sequence
  Description of Artificial Sequence Synthetic oligonucleotide
tttagaaatgactatgccc

SEQ ID NO: 368 - DNA, 19 bp, Artificial Sequence
  Description of Artificial Sequence Synthetic oligonucleotide
tactttctactagtgactt

SEQ ID NO: 369 - DNA, 19 bp, Artificial Sequence
  Description of Artificial Sequence Synthetic oligonucleotide
atactttctactagtgact

SEQ ID NO: 370 - DNA, 19 bp, Artificial Sequence
  Description of Artificial Sequence Synthetic oligonucleotide
tatactttctactagtgac

SEQ ID NO: 371 - DNA, 19 bp, Artificial Sequence
  Description of Artificial Sequence Synthetic oligonucleotide
ctcattcctccttccttcc

SEQ ID NO: 372 - DNA, 19 bp, Artificial Sequence
  Description of Artificial Sequence Synthetic oligonucleotide
actcattcctccttccttc

SEQ ID NO: 373 - DNA, 19 bp, Artificial Sequence
  Description of Artificial Sequence Synthetic oligonucleotide
tcactcattcctccttcct

SEQ ID NO: 374 - DNA, 19 bp, Artificial Sequence
  Description of Artificial Sequence Synthetic oligonucleotide
gtcactcattcctccttcc

SEQ ID NO: 375 - DNA, 19 bp, Artificial Sequence
  Description of Artificial Sequence Synthetic oligonucleotide
agtctctcttcaattaggt

SEQ ID NO: 376 - DNA, 19 bp, Artificial Sequence
  Description of Artificial Sequence Synthetic oligonucleotide
actctgtagtagtctctct

SEQ ID NO: 377 - DNA, 19 bp, Artificial Sequence
  Description of Artificial Sequence Synthetic oligonucleotide
cactctgtagtagtctctc

SEQ ID NO: 378 - DNA, 19 bp, Artificial Sequence
  Description of Artificial Sequence Synthetic oligonucleotide
atatcctaaccgccacttt

SEQ ID NO: 379 - DNA, 19 bp, Artificial Sequence
  Description of Artificial Sequence Synthetic oligonucleotide
atatatcctaaccgccact

SEQ ID NO: 380 - DNA, 19 bp, Artificial Sequence
  Description of Artificial Sequence Synthetic oligonucleotide
aatatatcctaaccgccac

SEQ ID NO: 381 - DNA, 19 bp, Artificial Sequence
  Description of Artificial Sequence Synthetic oligonucleotide
agttctgtcctctatttct

SEQ ID NO: 382 - DNA, 19 bp, Artificial Sequence
  Description of Artificial Sequence Synthetic oligonucleotide
tagttctgtcctctatttc

SEQ ID NO: 383 - DNA, 19 bp, Artificial Sequence
  Description of Artificial Sequence Synthetic oligonucleotide
tctagttctgtcctctatt

SEQ ID NO: 384 - DNA, 19 bp, Artificial Sequence
  Description of Artificial Sequence Synthetic oligonucleotide
tcagtctagttctgtcctc

SEQ ID NO: 385 - DNA, 19 bp, Artificial Sequence
  Description of Artificial Sequence Synthetic oligonucleotide
tatcagtctagttctgtcc

SEQ ID NO: 386 - DNA, 19 bp, Artificial Sequence
  Description of Artificial Sequence Synthetic oligonucleotide
ctatcagtctagttctgtc

SEQ ID NO: 387 - DNA, 19 bp, Artificial Sequence
  Description of Artificial Sequence Synthetic oligonucleotide
tgttctaggtcactgctat

SEQ ID NO: 388 - DNA, 19 bp, Artificial Sequence
  Description of Artificial Sequence Synthetic oligonucleotide
attgttctaggtcactgct

SEQ ID NO: 389 - DNA, 19 bp, Artificial Sequence
  Description of Artificial Sequence Synthetic oligonucleotide
aattgttctaggtcactgc

SEQ ID NO: 390 - DNA, 19 bp, Artificial Sequence
  Description of Artificial Sequence Synthetic oligonucleotide
tgtatccacataaatcctt

SEQ ID NO: 391 - DNA, 19 bp, Artificial Sequence
  Description of Artificial Sequence Synthetic oligonucleotide
ttgtatccacataaatcct

SEQ ID NO: 392 - DNA, 19 bp, Artificial Sequence
  Description of Artificial Sequence Synthetic oligonucleotide
tttgtatccacataaatcc

SEQ ID NO: 393 - DNA, 19 bp, Artificial Sequence
  Description of Artificial Sequence Synthetic oligonucleotide
ataaagtaattcactgctc

SEQ ID NO: 394 - DNA, 19 bp, Artificial Sequence
  Description of Artificial Sequence Synthetic oligonucleotide
ttgtttaagtgtttggtcc

SEQ ID NO: 395 - DNA, 19 bp, Artificial Sequence
  Description of Artificial Sequence Synthetic oligonucleotide
tttgtttaagtgtttggtc

SEQ ID NO: 396 - DNA, 19 bp, Artificial Sequence
  Description of Artificial Sequence Synthetic oligonucleotide
aatatgagacaagcttcct

SEQ ID NO: 397 - DNA, 19 bp, Artificial Sequence
  Description of Artificial Sequence Synthetic oligonucleotide
tgaatatgagacaagcttc

SEQ ID NO: 398 - DNA, 19 bp, Artificial Sequence
  Description of Artificial Sequence Synthetic oligonucleotide
agtgaatatgagacaagct

SEQ ID NO: 399 - DNA, 19 bp, Artificial Sequence
  Description of Artificial Sequence Synthetic oligonucleotide
gagtgaatatgagacaagc

SEQ ID NO: 400 - DNA, 19 bp, Artificial Sequence
  Description of Artificial Sequence Synthetic oligonucleotide
aataaacctaggactggat

SEQ ID NO: 401 - DNA, 19 bp, Artificial Sequence
  Description of Artificial Sequence Synthetic oligonucleotide
atatgaggctgaataactt

SEQ ID NO: 402 - DNA, 19 bp, Artificial Sequence
  Description of Artificial Sequence Synthetic oligonucleotide
attagattgttctgttccc

SEQ ID NO: 403 - DNA, 19 bp, Artificial Sequence
  Description of Artificial Sequence Synthetic oligonucleotide
cattagattgttctgttcc

SEQ ID NO: 404 - DNA, 19 bp, Artificial Sequence
  Description of Artificial Sequence Synthetic oligonucleotide
acattagattgttctgttc

SEQ ID NO: 405 - DNA, 19 bp, Artificial Sequence
  Description of Artificial Sequence Synthetic oligonucleotide
gctacatactggataagcc

SEQ ID NO: 406 - DNA, 19 bp, Artificial Sequence
  Description of Artificial Sequence Synthetic oligonucleotide
aaatagctacatactggat

SEQ ID NO: 407 - DNA, 18 bp, Artificial Sequence
  Description of Artificial Sequence Synthetic oligonucleotide
ccactcccagttctccgc

SEQ ID NO: 408 - DNA, 18 bp, Artificial Sequence
  Description of Artificial Sequence Synthetic oligonucleotide
cactgtcgtcgaatggcc

SEQ ID NO: 409 - DNA, 18 bp, Artificial Sequence
  Description of Artificial Sequence Synthetic oligonucleotide
acactgtcgtcgaatggc

SEQ ID NO: 410 - DNA, 18 bp, Artificial Sequence
  Description of Artificial Sequence Synthetic oligonucleotide
aatgaattcctttacacc

SEQ ID NO: 411 - DNA, 18 bp, Artificial Sequence
  Description of Artificial Sequence Synthetic oligonucleotide
aatacatccatggctaat

SEQ ID NO: 412 - DNA, 18 bp, Artificial Sequence
  Description of Artificial Sequence Synthetic oligonucleotide
acaactccctccttggcc

SEQ ID NO: 413 - DNA, 18 bp, Artificial Sequence
  Description of Artificial Sequence Synthetic oligonucleotide
tttctcagcagcagccac

SEQ ID NO: 414 - DNA, 18 bp, Artificial Sequence
  Description of Artificial Sequence Synthetic oligonucleotide
tgtctttcctgctgcttc

SEQ ID NO: 415 - DNA, 18 bp, Artificial Sequence
  Description of Artificial Sequence Synthetic oligonucleotide
ttgtctttcctgctgctt

SEQ ID NO: 416 - DNA, 18 bp, Artificial Sequence
  Description of Artificial Sequence Synthetic oligonucleotide
tttgtctttcctgctgct

SEQ ID NO: 417 - DNA, 18 bp, Artificial Sequence
  Description of Artificial Sequence Synthetic oligonucleotide
ttctcagccactgttgcc

SEQ ID NO: 418 - DNA, 18 bp, Artificial Sequence
  Description of Artificial Sequence Synthetic oligonucleotide
cttctcagccactgttgc

SEQ ID NO: 419 - DNA, 18 bp, Artificial Sequence
  Description of Artificial Sequence Synthetic oligonucleotide
ctctttggtcttctcagc

SEQ ID NO: 420 - DNA, 18 bp, Artificial Sequence
  Description of Artificial Sequence Synthetic oligonucleotide
tcacttgctctttggtct

SEQ ID NO: 421 - DNA, 18 bp, Artificial Sequence
  Description of Artificial Sequence Synthetic oligonucleotide
gtcacttgctctttggtc

SEQ ID NO: 422 - DNA, 18 bp, Artificial Sequence
  Description of Artificial Sequence Synthetic oligonucleotide
tgtcacttgctctttggt

SEQ ID NO: 423 - DNA, 18 bp, Artificial Sequence
  Description of Artificial Sequence Synthetic oligonucleotide
acatttgtcacttgctct

SEQ ID NO: 424 - DNA, 18 bp, Artificial Sequence
  Description of Artificial Sequence Synthetic oligonucleotide
aacatttgtcacttgctc

SEQ ID NO: 425 - DNA, 18 bp, Artificial Sequence
  Description of Artificial Sequence Synthetic oligonucleotide
tgtcttctgggctactgc

SEQ ID NO: 426 - DNA, 18 bp, Artificial Sequence
  Description of Artificial Sequence Synthetic oligonucleotide
tatcttccagaattcctt

SEQ ID NO: 427 - DNA, 18 bp, Artificial Sequence
  Description of Artificial Sequence Synthetic oligonucleotide
atatcttccagaattcct

SEQ ID NO: 428 - DNA, 18 bp, Artificial Sequence
  Description of Artificial Sequence Synthetic oligonucleotide
catatcttccagaattcc

SEQ ID NO: 429 - DNA, 18 bp, Artificial Sequence
  Description of Artificial Sequence Synthetic oligonucleotide
ttgtcaggatccacaggc

SEQ ID NO: 430 - DNA, 18 bp, Artificial Sequence
  Description of Artificial Sequence Synthetic oligonucleotide
ctcattgtcaggatccac

SEQ ID NO: 431 - DNA, 18 bp, Artificial Sequence
  Description of Artificial Sequence Synthetic oligonucleotide
gtcttgatacccttcctc

SEQ ID NO: 432 - DNA, 18 bp, Artificial Sequence
  Description of Artificial Sequence Synthetic oligonucleotide
tagtcttgatacccttcc

SEQ ID NO: 433 - DNA, 18 bp, Artificial Sequence
  Description of Artificial Sequence Synthetic oligonucleotide
gtagtcttgatacccttc

SEQ ID NO: 434 - DNA, 18 bp, Artificial Sequence
  Description of Artificial Sequence Synthetic oligonucleotide
tcgtagtcttgataccct

SEQ ID NO: 435 - DNA, 18 bp, Artificial Sequence
  Description of Artificial Sequence Synthetic oligonucleotide
ttcttaggcttcaggttc

SEQ ID NO: 436 - DNA, 18 bp, Artificial Sequence
  Description of Artificial Sequence Synthetic oligonucleotide
tttcttaggcttcaggtt

SEQ ID NO: 437 - DNA, 18 bp, Artificial Sequence
  Description of Artificial Sequence Synthetic oligonucleotide
atttcttaggcttcaggt

SEQ ID NO: 438 - DNA, 18 bp, Artificial Sequence
  Description of Artificial Sequence Synthetic oligonucleotide
agatatttcttaggcttc

SEQ ID NO: 439 - DNA, 18 bp, Artificial Sequence
  Description of Artificial Sequence Synthetic oligonucleotide
tctcaagaaactgggagc

SEQ ID NO: 440 - DNA, 18 bp, Artificial Sequence
  Description of Artificial Sequence Synthetic oligonucleotide
ttgtacaggatggaacat

SEQ ID NO: 441 - DNA, 18 bp, Artificial Sequence
  Description of Artificial Sequence Synthetic oligonucleotide
acttgtacaggatggaac

SEQ ID NO: 442 - DNA, 18 bp, Artificial Sequence
  Description of Artificial Sequence Synthetic oligonucleotide
cacattggaactgagcac

SEQ ID NO: 443 - DNA, 18 bp, Artificial Sequence
  Description of Artificial Sequence Synthetic oligonucleotide
tcactgctgatggaagac

SEQ ID NO: 444 - DNA, 18 bp, Artificial Sequence
  Description of Artificial Sequence Synthetic oligonucleotide
agatacttcaatcactgc

SEQ ID NO: 445 - DNA, 18 bp, Artificial Sequence
  Description of Artificial Sequence Synthetic oligonucleotide
acagatacttcaatcact

SEQ ID NO: 446 - DNA, 18 bp, Artificial Sequence
  Description of Artificial Sequence Synthetic oligonucleotide
ggtacagatacttcaatc

SEQ ID NO: 447 - DNA, 18 bp, Artificial Sequence
  Description of Artificial Sequence Synthetic oligonucleotide
cagtgaaagggaagcacc

SEQ ID NO: 448 - DNA, 18 bp, Artificial Sequence
  Description of Artificial Sequence Synthetic oligonucleotide
tcagtgaaagggaagcac

SEQ ID NO: 449 - DNA, 18 bp, Artificial Sequence
  Description of Artificial Sequence Synthetic oligonucleotide
tggtagtcacttaggtgt

SEQ ID NO: 450 - DNA, 18 bp, Artificial Sequence
  Description of Artificial Sequence Synthetic oligonucleotide
atagtttcatgctcacat

SEQ ID NO: 451 - DNA, 18 bp, Artificial Sequence
  Description of Artificial Sequence Synthetic oligonucleotide
ttataggtgcatagtttc

SEQ ID NO: 452 - DNA, 18 bp, Artificial Sequence
  Description of Artificial Sequence Synthetic oligonucleotide
attaaagtgagatgggat

SEQ ID NO: 453 - DNA, 18 bp, Artificial Sequence
  Description of Artificial Sequence Synthetic oligonucleotide
gttcttaattcatgttgc

SEQ ID NO: 454 - DNA, 18 bp, Artificial Sequence
  Description of Artificial Sequence Synthetic oligonucleotide
gtgtagggttaatgttcc

SEQ ID NO: 455 - DNA, 18 bp, Artificial Sequence
  Description of Artificial Sequence Synthetic oligonucleotide
agtgtagggttaatgttc

SEQ ID NO: 456 - DNA, 18 bp, Artificial Sequence
  Description of Artificial Sequence Synthetic oligonucleotide
agtgttgcttcagggaat

SEQ ID NO: 457 - DNA, 18 bp, Artificial Sequence
  Description of Artificial Sequence Synthetic oligonucleotide
acttctggcagtgttgct

SEQ ID NO: 458 - DNA, 18 bp, Artificial Sequence
  Description of Artificial Sequence Synthetic oligonucleotide
taattaatcacagccact

SEQ ID NO: 459 - DNA, 18 bp, Artificial Sequence
  Description of Artificial Sequence Synthetic oligonucleotide
ataattaatcacagccac

SEQ ID NO: 460 - DNA, 18 bp, Artificial Sequence
  Description of Artificial Sequence Synthetic oligonucleotide
caataattaatcacagcc

SEQ ID NO: 461 - DNA, 18 bp, Artificial Sequence
  Description of Artificial Sequence Synthetic oligonucleotide
atagtagttggggtcttc

SEQ ID NO: 462 - DNA, 18 bp, Artificial Sequence
  Description of Artificial Sequence Synthetic oligonucleotide
aatagtagttggggtctt

SEQ ID NO: 463 - DNA, 18 bp, Artificial Sequence
  Description of Artificial Sequence Synthetic oligonucleotide
tacaatagtagttggggt

SEQ ID NO: 464 - DNA, 18 bp, Artificial Sequence
  Description of Artificial Sequence Synthetic oligonucleotide
tcagaaaggtacagcatt

SEQ ID NO: 465 - DNA, 18 bp, Artificial Sequence
  Description of Artificial Sequence Synthetic oligonucleotide
ttgtcagaaaggtacagc

SEQ ID NO: 466 - DNA, 18 bp, Artificial Sequence
  Description of Artificial Sequence Synthetic oligonucleotide
ctacactgcttagttccc

SEQ ID NO: 467 - DNA, 18 bp, Artificial Sequence
  Description of Artificial Sequence Synthetic oligonucleotide
tctacactgcttagttcc

SEQ ID NO: 468 - DNA, 18 bp, Artificial Sequence
  Description of Artificial Sequence Synthetic oligonucleotide
ttctacactgcttagttc

SEQ ID NO: 469 - DNA, 18 bp, Artificial Sequence
  Description of Artificial Sequence Synthetic oligonucleotide
tcatcttctacactgctt

SEQ ID NO: 470 - DNA, 18 bp, Artificial Sequence
  Description of Artificial Sequence Synthetic oligonucleotide
atcatcttctacactgct

SEQ ID NO: 471 - DNA, 18 bp, Artificial Sequence
  Description of Artificial Sequence Synthetic oligonucleotide
aatcatcttctacactgc

SEQ ID NO: 472 - DNA, 18 bp, Artificial Sequence
  Description of Artificial Sequence Synthetic oligonucleotide
gaatgtgctaatatgtgc

SEQ ID NO: 473 - DNA, 18 bp, Artificial Sequence
  Description of Artificial Sequence Synthetic oligonucleotide
tcagagccttgaatgtgc

SEQ ID NO: 474 - DNA, 18 bp, Artificial Sequence
  Description of Artificial Sequence Synthetic oligonucleotide
tttaaaggcatttcctgt

SEQ ID NO: 475 - DNA, 18 bp, Artificial Sequence
  Description of Artificial Sequence Synthetic oligonucleotide
tgtttaaaggcatttcct

SEQ ID NO: 476 - DNA, 18 bp, Artificial Sequence
  Description of Artificial Sequence Synthetic oligonucleotide
atgtttaaaggcatttcc

SEQ ID NO: 477 - DNA, 18 bp, Artificial Sequence
  Description of Artificial Sequence Synthetic oligonucleotide
gatgtttaaaggcatttc

SEQ ID NO: 478 - DNA, 18 bp, Artificial Sequence
  Description of Artificial Sequence Synthetic oligonucleotide
gactctggtagttccaac

SEQ ID NO: 479 - DNA, 18 bp, Artificial Sequence
  Description of Artificial Sequence Synthetic oligonucleotide
agtctagagaattgatct

SEQ ID NO: 480 - DNA, 18 bp, Artificial Sequence
  Description of Artificial Sequence Synthetic oligonucleotide
cagtctagagaattgatc

SEQ ID NO: 481 - DNA, 18 bp, Artificial Sequence
  Description of Artificial Sequence Synthetic oligonucleotide
acatttaaaggaggccat

SEQ ID NO: 482 - DNA, 18 bp, Artificial Sequence
  Description of Artificial Sequence Synthetic oligonucleotide
ggagttaatagatcttcc

SEQ ID NO: 483 - DNA, 18 bp, Artificial Sequence
  Description of Artificial Sequence Synthetic oligonucleotide
ttagaaatgactatgccc

SEQ ID NO: 484 - DNA, 18 bp, Artificial Sequence
  Description of Artificial Sequence Synthetic oligonucleotide
tttagaaatgactatgcc

SEQ ID NO: 485 - DNA, 18 bp, Artificial Sequence
  Description of Artificial Sequence Synthetic oligonucleotide
tactttctactagtgact

SEQ ID NO: 486 - DNA, 18 bp, Artificial Sequence
  Description of Artificial Sequence Synthetic oligonucleotide
atactttctactagtgac

SEQ ID NO: 487 - DNA, 18 bp, Artificial Sequence
  Description of Artificial Sequence Synthetic oligonucleotide
ctcattcctccttccttc

SEQ ID NO: 488 - DNA, 18 bp, Artificial Sequence
  Description of Artificial Sequence Synthetic oligonucleotide
actcattcctccttcctt

SEQ ID NO: 489 - DNA, 18 bp, Artificial Sequence
  Description of Artificial Sequence Synthetic oligonucleotide
tcactcattcctccttcc

SEQ ID NO: 490 - DNA, 18 bp, Artificial Sequence
  Description of Artificial Sequence Synthetic oligonucleotide
gtcactcattcctccttc

SEQ ID NO: 491 - DNA, 18 bp, Artificial Sequence
  Description of Artificial Sequence Synthetic oligonucleotide
gaagtttctatggtaacc

SEQ ID NO: 492 - DNA, 18 bp, Artificial Sequence
  Description of Artificial Sequence Synthetic oligonucleotide
actctgtagtagtctctc

SEQ ID NO: 493 - DNA, 18 bp, Artificial Sequence
  Description of Artificial Sequence Synthetic oligonucleotide
ttctaaccttcctgaaat

SEQ ID NO: 494 - DNA, 18 bp, Artificial Sequence
  Description of Artificial Sequence Synthetic oligonucleotide
atatcctaaccgccactt

SEQ ID NO: 495 - DNA, 18 bp, Artificial Sequence
  Description of Artificial Sequence Synthetic oligonucleotide
atatatcctaaccgccac

SEQ ID NO: 496 - DNA, 18 bp, Artificial Sequence
  Description of Artificial Sequence Synthetic oligonucleotide
aaatatatcctaaccgcc

SEQ ID NO: 497 - DNA, 18 bp, Artificial Sequence
  Description of Artificial Sequence Synthetic oligonucleotide
aaatatgctgctttaggt

SEQ ID NO: 498 - DNA, 18 bp, Artificial Sequence
  Description of Artificial Sequence Synthetic oligonucleotide
ttctgtcctctatttctt

SEQ ID NO: 499 - DNA, 18 bp, Artificial Sequence
  Description of Artificial Sequence Synthetic oligonucleotide
agttctgtcctctatttc

SEQ ID NO: 500 - DNA, 18 bp, Artificial Sequence
  Description of Artificial Sequence Synthetic oligonucleotide
tagttctgtcctctattt

SEQ ID NO: 501 - DNA, 18 bp, Artificial Sequence
  Description of Artificial Sequence Synthetic oligonucleotide
agtctagttctgtcctct

SEQ ID NO: 502 - DNA, 18 bp, Artificial Sequence
  Description of Artificial Sequence Synthetic oligonucleotide
cagtctagttctgtcctc

SEQ ID NO: 503 - DNA, 18 bp, Artificial Sequence
  Description of Artificial Sequence Synthetic oligonucleotide
tcagtctagttctgtcct

SEQ ID NO: 504 - DNA, 18 bp, Artificial Sequence
  Description of Artificial Sequence Synthetic oligonucleotide
tatcagtctagttctgtc

SEQ ID NO: 505 - DNA, 18 bp, Artificial Sequence
  Description of Artificial Sequence Synthetic oligonucleotide
ttgttctaggtcactgct

SEQ ID NO: 506 - DNA, 18 bp, Artificial Sequence
  Description of Artificial Sequence Synthetic oligonucleotide
attgttctaggtcactgc

SEQ ID NO: 507 - DNA, 18 bp, Artificial Sequence
  Description of Artificial Sequence Synthetic oligonucleotide
tgtatccacataaatcct

SEQ ID NO: 508 - DNA, 18 bp, Artificial Sequence
  Description of Artificial Sequence Synthetic oligonucleotide
ttgtatccacataaatcc

SEQ ID NO: 509 - DNA, 18 bp, Artificial Sequence
  Description of Artificial Sequence Synthetic oligonucleotide
aggagaatttgtatccac

SEQ ID NO: 510 - DNA, 18 bp, Artificial Sequence
  Description of Artificial Sequence Synthetic oligonucleotide
taaagtaattcactgctc

SEQ ID NO: 511 - DNA, 18 bp, Artificial Sequence
  Description of Artificial Sequence Synthetic oligonucleotide
ttgtttaagtgtttggtc

SEQ ID NO: 512 - DNA, 18 bp, Artificial Sequence
  Description of Artificial Sequence Synthetic oligonucleotide
aatatgagacaagcttcc

SEQ ID NO: 513 - DNA, 18 bp, Artificial Sequence
  Description of Artificial Sequence Synthetic oligonucleotide
tgaatatgagacaagctt

SEQ ID NO: 514 - DNA, 18 bp, Artificial Sequence
  Description of Artificial Sequence Synthetic oligonucleotide
agtgaatatgagacaagc

SEQ ID NO: 515 - DNA, 18 bp, Artificial Sequence
  Description of Artificial Sequence Synthetic oligonucleotide
aatgtctcgggagtgaat

SEQ ID NO: 516 - DNA, 18 bp, Artificial Sequence
  Description of Artificial Sequence Synthetic oligonucleotide
attgatcctcaggccact

SEQ ID NO: 517 - DNA, 18 bp, Artificial Sequence
  Description of Artificial Sequence Synthetic oligonucleotide
gattgatcctcaggccac

SEQ ID NO: 518 - DNA, 18 bp, Artificial Sequence
  Description of Artificial Sequence Synthetic oligonucleotide
aataacttgggagaatgt

SEQ ID NO: 519 - DNA, 18 bp, Artificial Sequence
  Description of Artificial Sequence Synthetic oligonucleotide
ttagattgttctgttccc

SEQ ID NO: 520 - DNA, 18 bp, Artificial Sequence
  Description of Artificial Sequence Synthetic oligonucleotide
attagattgttctgttcc

SEQ ID NO: 521 - DNA, 18 bp, Artificial Sequence
  Description of Artificial Sequence Synthetic oligonucleotide
cattagattgttctgttc

SEQ ID NO: 522 - DNA, 18 bp, Artificial Sequence
  Description of Artificial Sequence Synthetic oligonucleotide
ctacatactggataagcc

SEQ ID NO: 523 - DNA, 18 bp, Artificial Sequence
  Description of Artificial Sequence Synthetic oligonucleotide
gctacatactggataagc

SEQ ID NO: 524 - DNA, 18 bp, Artificial Sequence
  Description of Artificial Sequence Synthetic oligonucleotide
aatagctacatactggat

SEQ ID NO: 525 - DNA, 21 bp, Artificial Sequence
  Description of Artificial Sequence Synthetic oligonucleotide
  misc_feature (1)..(21): Internucleoside linkage including a phosphorothioate linkage
  modified_base (1)..(6): Fluoro-modified nucleotide
  modified_base (16)..(21): Fluoro-modified nucleotide
uauauucccagctcccuccac

SEQ ID NO: 526 - DNA, 21 bp, Artificial Sequence
  Description of Artificial Sequence Synthetic oligonucleotide
  misc_feature (1)..(21): Internucleoside linkage including a phosphorothioate linkage
  modified_base (1)..(6): Fluoro-modified nucleotide
  modified_base (16)..(21): Fluoro-modified nucleotide
acaauutaagtgtgaagccac

SEQ ID NO: 527 - DNA, 21 bp, Artificial Sequence
  Description of Artificial Sequence Synthetic oligonucleotide
  misc_feature (1)..(21): Internucleoside linkage including a phosphorothioate linkage
  modified_base (1)..(6): Fluoro-modified nucleotide
  modified_base (16)..(21): Fluoro-modified nucleotide
aaccuacatagaggacucccu

SEQ ID NO: 528 - DNA, 21 bp, Artificial Sequence
  Description of Artificial Sequence Synthetic oligonucleotide
  misc_feature (1)..(21): Internucleoside linkage including a phosphorothioate linkage
  modified_base (1)..(6): Fluoro-modified nucleotide
  modified_base (16)..(21): Fluoro-modified nucleotide
uauauucccagctcccuccac

SEQ ID NO: 529 - DNA, 21 bp, Artificial Sequence
  Description of Artificial Sequence Synthetic oligonucleotide
  misc_feature (1)..(21): Internucleoside linkage including a phosphorothioate linkage
  modified_base (1)..(6): Fluoro-modified nucleotide
  modified_base (16)..(21): Fluoro-modified nucleotide
cuuucatgaacacatccaugg

SEQ ID NO: 530 - DNA, 23 bp, Artificial Sequence
  Description of Artificial Sequence Synthetic oligonucleotide
  misc_feature (1)..(23): Internucleoside linkage including a phosphorothioate linkage
  modified_base (1)..(6): Fluoro-modified nucleotide
  modified_base (18)..(23): Fluoro-modified nucleotide
ccuuucatgaacacatccauggc

SEQ ID NO: 531 - DNA, 21 bp, Artificial Sequence
  Description of Artificial Sequence Synthetic oligonucleotide
tatattcccagctccctccac

SEQ ID NO: 532 - DNA, 21 bp, Artificial Sequence
  Description of Artificial Sequence Synthetic oligonucleotide
acaatttaagtgtgaagccac

SEQ ID NO: 533 - DNA, 21 bp, Artificial Sequence
  Description of Artificial Sequence Synthetic oligonucleotide
aacctacatagaggactccct

SEQ ID NO: 534 - DNA, 21 bp, Artificial Sequence
  Description of Artificial Sequence Synthetic oligonucleotide
tatattcccagctccctccac

SEQ ID NO: 535 - DNA, 21 bp, Artificial Sequence
  Description of Artificial Sequence Synthetic oligonucleotide
ctttcatgaacacatccatgg

SEQ ID NO: 536 - DNA, 23 bp, Artificial Sequence
  Description of Artificial Sequence Synthetic oligonucleotide
cctttcatgaacacatccatggc

(19) United States
(12) Patent Application Publication
(10) Pub. No.: US 2024/0191228 A1
(43) Pub. Date: Jun. 13, 2024

(54) ANTISENSE OLIGONUCLEOTIDES FOR TREATMENT OF NEUROLOGICAL DISORDERS

(71) Applicants: AUM LifeTech, Inc., Philadelphia, PA (US); The Trustees of the University of Pennsylvania, Philadelphia, PA (US)

(72) Inventors: Veenu Aishwarya, Philadelphia, PA (US); Anna Caputo, Philadelphia, PA (US); Kelvin Cheuk Wai Luk, Philadelphia, PA (US)

(21) Appl. No.: 17/789,750

(22) PCT Filed: Jan. 5, 2021
(86) PCT No.: PCT/US2021/012208

§ 371 Date: Jun. 28, 2022

Related U.S. Application Data
(60) Provisional application No. 62/957,636, filed on Jan. 6, 2020.

(51) Int. Cl. C12N 15/113; A61P 25/00

(52) U.S. Cl. CPC C12N 15/113; A61P 25/00; C12N 2310/11; C12N 2310/322

(57) ABSTRACT

Parkinson's Disease (PD) is the second most common neurodegenerative disorder. Essentially all PD patients accumulate misfolded forms of alpha-synuclein (α-Syn) in their neurons, while mutations in the α-synuclein gene (SNCA) cause familial PD, suggesting that abnormal α-synuclein plays a central role in PD. The present invention is based on the seminal discovery that FANA antisense oligonucleotides targeting α-synuclein are effective at treating and/or preventing Parkinson's Disease. Specifically. FANA antisense oligonucleotides targeting α-synuclein decrease the expression of α-synuclein in neurons and decrease Lewy body and Lewy neurite pathology.

### CROSS-REFERENCE TO RELATED APPLICATIONS

[This application claims benefit of priority under 35 U.S.C. § 119(e) of U.S. Ser. No. 62/957,636, filed Jan. 6, 2020, the entire contents of which is incorporated herein by reference in its entirety.

### INCORPORATION OF SEQUENCE LISTING

The material in the accompanying sequence listing is hereby incorporated by reference into this application. The accompanying sequence listing text file, name AUM1230_1WO_Sequence_Listing.txt, was created on Dec. 29, 2020, and is 126 kb. The file can be accessed using Microsoft Word on a computer that uses Windows OS.

### BACKGROUND OF THE INVENTION

### Field of the Invention

The present invention relates generally to the prevention and treatment of neurological diseases and more specifically to the use of antisense oligonucleotides to target intracellular α-synuclein.

### Background Information

Parkinson's Disease (PD) is the second most common neurodegenerative disorder that affects approximately 1% of the >60-year old population and for which there is no disease-modifying therapy. Characterized mainly by motor symptoms (bradykinesia, tremor, rigidity, and postural instability) that occur mostly due to the degeneration of substantia nigra pars compacta (SNpc) dopaminergic (DA) neurons, selective cell loss in other CNS regions (e.g. locus coeruleus, dorsal Raphe nucleus, vagal dorsal motor nucleus) also occurs in PD, giving rise to a variety of non-motor symptoms (e.g. hyposmia, autonomic dysfunction, depression, hallucinations, and sleep disturbances). Up to 40% of PD patients also develop cognitive impairments and those with late-stage disease show a high prevalence (>80%) of dementia (designated as PDD) although this may also be associated with co-morbid Alzheimer's disease (AD) in about a third of cases. However, the mechanisms that lead to this non-uniform pattern of cell loss and characteristic symptomology are poorly understood.

Lewy bodies (LBs) and Lewy neurites (LNs) are the neuropathological hallmarks of PD, PDD and dementia with Lewy bodies (DLB), a related disorder distinguished by the onset of dementia prior to classical Parkinsonism. These intraneuronal inclusions are comprised of aggregated α-synuclein, a heat-stable 140 amino acid long protein expressed ubiquitously in a variety of tissues including neurons and erythrocytes. Importantly, point mutations or amplification of the gene encoding α-synuclein (SNCA) cause autosomal dominant forms of familial PD. Moreover, α-synuclein also forms glial cell inclusions within oligodendrocytes of patients with multiple systems atrophy (MSA). Thus, histological and genetic evidence collectively point to the accumulation of abnormal α-synuclein as a central step in the pathogenesis of these neurodegenerative disorders (NDDs). Indeed, LBs/LNs are present in the brains of nearly all patients with sporadic and/or familial PD. The function of α-synuclein is not fully known, but its enrichment at presynaptic terminals points to a role in regulating synaptic vesicle formation and neurotransmitter release. In contrast to its highly soluble state in healthy brains, α-synuclein in LBs/LNs exist as β-sheet-rich amyloid fibrils, an ultrastructural arrangement shared by proteins that accumulate in several other major NDDs including AD, polyglutamine-expansion diseases, and transmissible spongiform encephalopathies (i.e. prion diseases). Recombinant α-synuclein, which has no native secondary structure, also assembles into fibrils at micromolar concentrations. α-synuclein recovered from PD brains is further characterized by insolubility to detergents, and various post-translational modifications including proteolytic cleavage, hyperphosphorylation (e.g., Ser129), ubiquitination, nitration and oxidation. Thus, histological and genetic evidence strongly point to the accumulation of abnormal α-synuclein as a central step in the pathogenesis of multiple disorders (PD, PDD, DLB, MSA, and AD) and point to α-synuclein as a potential target for novel disease-modifying therapies.

The current therapies in clinical trials for PD include antibody and small molecule approaches targeting both toxic and non-toxic forms of α-synuclein. Such approaches mainly target these proteins at the extracellular level and thus may have limited therapeutic benefits. Reduction of α-synuclein expression is neuroprotective in multiple experimental models of PD, indicating its potential as a disease-modifying therapy. Gene silencing antisense oligonucleotide (ASO) therapy may overcome these limitations by directly targeting intracellular α-synuclein and thus reducing formation of pathological α-synuclein species. A gene silencing therapy was developed that utilizes self-deliverable 2′-deoxy-2′-fluoro-D-arabinonucleic acid antisense oligonucleotides (FANA-ASOs) which can be effectively delivered in vivo and selectively inhibit production of α-synuclein by knocking down SNCA gene.

### SUMMARY OF THE INVENTION

The present invention is based on the seminal discovery that 2′-deoxy-2′-fluoro-D-arabinonucleic acid antisense oligonucleotides (FANA-ASOs) targeting α-synuclein are effective at decreasing the expression of α-synuclein. Specifically, FANA-ASO oligonucleotides targeting α-synuclein decrease the expression of α-synuclein in neurons and decrease Lewy body (LB) and Lewy neurite (LN) pathology and may be effective for treating α-synucleinpathies such as Parkinson's Disease.

As described herein FANA-ASOs may be useful for the prevention and/or treatment of Parkinson's Disease by decreasing the expression of α-synuclein in neurons and decreasing Lewy body (LB) and Lewy neurite (LN) pathology. By effectively reducing the levels of α-synuclein it is expected that there will be a reduction of α-synuclein pathology formation and improved neuronal function; prevention of dopaminergic cell loss and dysfunction; improvement in survival of glutamatergic, serotonergic and cholinergic neurons; and extended inhibition of SNCA to reduce established aggregate pathology and prevents dopamine neuron loss, for example.

In one embodiment, the present invention provides a composition with an α-synuclein targeting FANA-ASO oligonucleotide. In one aspect, the α-synuclein targeting FANA-ASO oligonucleotide has a nucleic acid sequence selected from SEQ ID NOs: 1-536 or a combination thereof. In an additional aspect, the α-synuclein targeting FANA-ASO oligonucleotide has at least one 2′FANA modified nucleotide. In a further aspect, the at least one 2′FANA modified nucleotide is positioned within the oligonucleotide according to any of Formula 1-16.

In an additional embodiment, the present invention provides a pharmaceutical composition with an α-synuclein targeting FANA-ASO oligonucleotide and a pharmaceutically acceptable carrier. In one aspect, the α-synuclein targeting FANA-ASO oligonucleotide has a nucleic acid sequence of SEQ ID NOs: 1-536 or a combination thereof. In an additional aspect, the α-synuclein targeting FANA-ASO oligonucleotide has at least one 2′FANA modified nucleotide. In a further aspect, the at least one 2′FANA modified nucleotide is positioned within the oligonucleotide according to any of Formula 1-16. In certain aspects, the pharmaceutically acceptable carrier is phosphate buffer; citrate buffer; ascorbic acid; methionine; octadecyldimethylbenzyl ammonium chloride; hexamethonium chloride; benzalkonium chloride; benzethonium chloride; phenol alcohol; butyl alcohol; benzyl alcohol; methyl paraben; propyl paraben; catechol; resorcinol; cyclohexanol; 3-pentanol; m-cresol; low molecular weight (less than about 10 residues) polypeptides; serum albumin; gelatin; immunoglobulins; polyvinylpyrrolidone glycine; glutamine; asparagine; histidine; arginine; lysine; monosaccharides; disaccharides; glucose; mannose; dextrins; EDTA; sucrose; mannitol; trehalose; sorbitol; sodium; saline; metal surfactants; non-ionic surfactants; polyethylene glycol (PEG); magnesium stearate; water; alcohol; saline solution; glycol; mineral oil or dimethyl sulfoxide (DMSO).

In a further embodiment, the present invention provides a method of decreasing α-synuclein expression by administering an α-synuclein targeting FANA-ASO oligonucleotide to a subject in need thereof, thereby reducing α-synuclein expression. In one aspect, the α-synuclein expression is decreased in neurons, oligodendrocytes and/or astrocytes. In an additional aspect, the α-synuclein targeting FANA-ASO oligonucleotide has at least one 2′FANA modified nucleotide. In certain aspects, the at least one 2′FANA modified nucleotide is positioned within the oligonucleotide according to any of Formula 1-16. In various aspects, the α-synuclein targeting FANA-ASO oligonucleotide has a nucleic acid sequence of SEQ ID NOs: 1-536 or a combination thereof. In a further aspect, the α-synuclein targeting FANA-ASO oligonucleotide has the nucleic acid sequence of SEQ ID NOs:525 or 527. In certain aspects, the α-synuclein targeting FANA-ASO oligonucleotide is administered by intracutaneous, subcutaneous, intravenous, intraperitoneal, intraarterial, intrathecal, intracapsular, intraorbital, intracardiac, intradermal, transdermal, transtracheal, subcuticular, intraarticular, intracerebroventricular, subcapsular, subarachnoid, intraspinal, intrasternal, oral, sublingual buccal, rectal, vaginal, ocular, inhalation, or nebulization.

In another embodiment, the present invention provides a method of reducing Lewy body and/or Lewy neurite pathology by administering an α-synuclein targeting FANA-ASO oligonucleotide to a subject in need thereof, thereby decreasing Lewy body and/or Lewy neurite pathology. In one aspect, the reduction of the Lewy body and/or Lewy neurite pathology is in neurons, oligodendrocytes and/or astrocytes. In an additional aspect, the α-synuclein targeting FANA-ASO oligonucleotide has at least one 2′FANA modified nucleotide. In certain aspects, the at least 2′FANA modified nucleotide is positioned within the oligonucleotide according to any of Formula 1-16. In various aspects, the α-synuclein targeting FANA-ASO oligonucleotide has a nucleic acid sequence of SEQ ID NOs:1-536 or a combination thereof. In a further aspect, the α-synuclein targeting FANA-ASO oligonucleotide has the nucleic acid sequence of SEQ ID NOs: 525 or 527.

In one embodiment, the present invention provides a method of preventing and/or treating Parkinson's Disease or symptoms thereof, by administering an α-synuclein targeting FANA-ASO oligonucleotide to a subject in need thereof, thereby preventing and/or treating Parkinson's Disease. In one aspect, the administration of the α-synuclein targeting FANA-ASO oligonucleotide decreases expression of α-synuclein in cells. In certain aspects, the cells are neurons, oligodendrocytes and/or astrocytes. In various aspects, the α-synuclein targeting FANA-ASO oligonucleotide is administered by intracutaneous, subcutaneous, intravenous, intraperitoneal, intraarterial, intrathecal, intracapsular, intraorbital, intracardiac, intradermal, transdermal, transtracheal, subcuticular, intraarticular, intracerebroventricular, subcapsular, subarachnoid, intraspinal, intrasternal, oral, sublingual buccal, rectal, vaginal, ocular, infusion, inhalation, or nebulization. In one aspect, the subject is human. In an additional aspect, the α-synuclein targeting FANA-ASO oligonucleotide has at least one 2′FANA modified nucleotide. In a further aspect, the at least one 2′FANA modified nucleotide is positioned within the oligonucleotide according to any of Formula 1-16. In certain aspects, the α-synuclein targeting FANA-ASO oligonucleotide has a nucleic acid sequence of SEQ ID NOs:1-536 or a combination thereof. In a further aspect, the α-synuclein targeting FANA-ASO oligonucleotide has the nucleic acid sequence of SEQ ID NOs:525 or 527. In another aspect, Lewy body and/or Lewy neurite pathology is reduced. In an additional aspect, a therapeutic agent is administered. In a further aspect, the therapeutic agent is administered prior to, simultaneously with, or following administration of the α-synuclein targeting FANA-ASO oligonucleotide. In a specific aspect, the therapeutic agent is Levodopa.

### BRIEF DESCRIPTION OF THE DRAWINGS

FIGS. 1A-1C show FANA-ASO mediated knockdown of α-synuclein-GFP in mouse neurons. FIG. 1A. Fluorescence image of α-synuclein-GFP levels in neurons treated with FANA-ASO sequences. FIG. 1B. Quantification of fluorescence levels of α-synuclein-GFP in neurons treated with different FANA-ASO sequences. FIG. 1C. Western blot quantification of α-synuclein-GFP levels in neurons treated with the indicated FANA-ASO sequences.

FIGS. 2A-2E show the distribution of FANA-ASO in mouse brain after intracerebroventral injection (i.c.v.). FIGS. 2A and 2B. Fluorescence images of the distribution of FANA-ASO sequences mouse brain. FIG. 2C. lack of signal in un-injected mouse brain. FIG. 2D. Distribution of FANA-ASO in the cerebral cortex and striatum of injected mice, as highlighted by white box in panel B. FIG. 2E. High power micrographs showing FANA-ASO within the cell bodies of NeuN-labeled neurons and also non-neuronal cells in the cerebral cortex.

FIGS. 3A-3B show FANA-ASO mediated knockdown of α-synuclein reduces fibril-induced Lewy-like pathology in neurons. FIG. 3A. Fluorescence image of cells treated with FANA-ASO sequences that show reduce fibril induced Lewy-like pathology. FIG. 3B. Quantification of α-synuclein levels in neurons co-treated with PFFs and either Syn3 or scrambled FANA-ASO.

FIG. 4 shows α-synuclein levels in mice following administration of FANA-ASO (syn3) targeting α-synuclein.

### DETAILED DESCRIPTION OF THE INVENTION

The present invention is based on the seminal discovery that 2′-deoxy-2′-fluoro-D-arabinonucleic acid antisense oligonucleotides (FANA-ASOs) targeting α-synuclein are effective at decreasing the expression of α-synuclein. Specifically, FANA-ASO oligonucleotides targeting α-synuclein decrease the expression of α-synuclein in neurons and decrease Lewy body (LB) and Lewy neurite (LN) pathology and may be effective for treating α-synuclein pathologies such as Parkinson's Disease.

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

All publications, patents, and patent applications mentioned in this specification are herein incorporated by reference to the same extent as if each individual publication, patent, or patent application was specifically and individually indicated to be incorporated by reference.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, it will be understood that modifications and variations are encompassed within the spirit and scope of the instant disclosure. The preferred methods and materials are now described.

In one embodiment, the present invention provides a composition with an α-synuclein targeting FANA-ASO oligonucleotide. In one aspect, the α-synuclein targeting FANA-ASO oligonucleotide has a nucleic acid sequence selected from SEQ ID NOs: 1-536 or a combination thereof. In an additional aspect, the α-synuclein targeting FANA-ASO oligonucleotide has at least one 2′FANA modified nucleotide. In a further aspect, the at least one 2′FANA modified nucleotide is positioned within the oligonucleotide according to any of Formula 1-16.

Alpha-synuclein (α-synuclein) is a protein that, in humans, is encoded by the SNCA gene that is abundant in the brain, while smaller amounts are found in the heart, muscle and other tissues. In the brain, α-synuclein is found mainly in neurons within presynaptic terminals. Although the function of alpha-synuclein is not well understood, studies suggest that it plays a role in restricting the mobility of synaptic vesicles, consequently attenuating synaptic vesicle recycling and neurotransmitter release. Human α-synuclein protein is made of 140 amino acids.

Antisense oligonucleotides (ASOs) are short synthetic oligonucleotides that inhibit or modulate expression of a specific gene by Watson-Crick binding to cellular RNA targets. ASOs act through a number of different mechanisms. Some ASOs bind to an mRNA of a gene of interest, inhibiting expression either by blocking access (steric blocker) of the cellular translation machinery, or by inducing its enzymatic degradation (RNAse-H, RNAse-P). Alternatively, ASOs can target a complementary region of a specific pre-mRNA and modulate its splicing, typically to correct a dysfunctional protein.

FANA (2′-Deoxy-2′-Fluoro-β-D-Arabinonucleic Acid) antisense oligonucleotides are nucleic acids with a phosphorothioate backbone and modified flanking nucleotides, in which the 2′-OH group of the ribose sugar was substituted by a fluorine atom. The flank modifications increase the resistance of the ASOs to degradation and enhance binding to targeted mRNA. The FANA/RNA duplex is recognized by ribonuclease H (RNase H), an enzyme that catalyzes the degradation of duplexed mRNA.

Antisense oligonucleotides of the present invention are single-stranded deoxyribonucleotides complementary to a targeted mRNA or DNA. Hybridization of an ASO to its target mRNA via Watson-Crick base pairing can result in specific inhibition of gene expression by various mechanisms, depending on the chemical make-up of the ASO and location of hybridization, resulting in reduced levels of translation of the target transcript (Crooke 2004). ASOs of the present invention typically encompass oligonucleotides having at least one sugar-modified nucleoside (e.g., 2′FANA) as well as naturally-occurring 2′-deoxy-nucleosides (see, e.g., U.S. Pat. No. 8,278,103 which is specifically incorporated by reference). ASO-induced protein knockdown is usually achieved by induction of RNase H endonuclease activity. When activated, the RNAse H cleaves the RNA-DNA heteroduplex leading to the degradation of the target mRNA. This leaves the ASO intact so that it can function again.

While there are many types of ASO's, the main discoveries in ASO development included two main chemical modifications. These modifications include the 2′-fluoro (2′-F) substitutions and the phosphorothioate chemistry. These two modifications constitute synthetic analogs of naturally occurring nucleic acids, but which have greater stability and activity. Thus, some embodiments of the present invention use 2′-F substitutions, and modification of the sugar backbone with phosphorothioate chemistry to produce ASOs containing 2′-deoxy-2′-fluoro-β-D-arabinonucleic acid (2′F-ANA), termed “FANA antisense oligonucleotides” (FANA-ASO).

FANA-ASOs are chemically modified single stranded synthetic nucleic acids with a phosphorothioate (PS) backbone and a 2′-fluorine that substitutes the hydroxyl group on the ribose sugar. The chemical modifications on the FANA-ASOs provide resistance to nucleases, increase target binding affinity, enhance the ASOs pharmacokinetic properties, and reduce immune response in vivo. The PS modification facilitated cellular uptake by increasing hydrophobicity and its high affinity for plasma proteins. This allows for the modified ASOs to slowly cross the lipid bilayer into the cytoplasm and nucleus, while escaping endosomes. In addition, this feature gives a key advantage to FANA-ASOs to be self-derivable.

Self-delivery is an important characteristic for a therapeutic agent because it avoids the need for additional formulations or delivery agents that can increase toxicity and manufacturing costs. FANA-ASOs can be delivered in animals by multiple modes of administration without the need of additional delivery agents. It has been shown that FANA-ASOs can be used to target genes across a wide spectrum of biological models. For example, FANAs have been delivered to T cells, neurons, and stem cells both in vitro and in vivo without triggering toxicity or an immune response. In addition to self-delivery ability of FANA-ASOs, these studies have shown potent and effective knockdown of a range of RNA targets; for example, mRNA, microRNA, and long non-coding RNA.

FANA-ASOs can also comprise a DNA segment flanked by FANA segments. When targeting RNA, these segments are arranged as either a ‘gapmer’ (F-DNA-F) or ‘altimer’ (F-DNA-F-DNA-F) configuration. Depending on their design, FANA-ASOs are made to be complementary to their RNA target and modulate RNA function by either tightly binding to RNA directly (steric blockers) or associating with an endonuclease (RNase H) to cleave RNA. FANA single-stranded antisense oligonucleotides can elicit RNase H to mediate RNA cleavage as opposed to the RNAi pathway that involves the RISC complex. The FANA-ASO first binds to the RNA target using highly specific Watson-Crick base pairing. RNase H then recognizes the RNA/DNA hybrid and cleaves the RNA within the hybrid. Following cleavage, the fragmented RNA is further degraded by nucleases and FANA-ASOs are recycled. One FANA-ASO can degrade many copies of RNA; thus, increasing efficiency and lowering the dosage requirement. The dual modification system of FANA-ASOs ensures that there is no non-specific hybridization. The dual modification system includes (1) backbone modification and (2) FANA modification on the sugar. This allows the Watson-crick base paring of FANA-ASOs with the target to be highly sequence specific. To this end, even if FANA-ASOs enter non-specific cells, they will cause no harm to those cells as they will not hybridize with any of the human endogenous genes and will eventually degrade.

### FANA Antisense Oligonucleotides

The chemistry and construction of 2′F-ANA oligonucleotides (also termed FANA or FANA-ASO) has been described elsewhere in detail (See, e.g., U.S. Pat. Nos. 8,278,103 and 9,902,953). The FANA-ASOs and methods of using them disclosed herein contemplate any FANA chemistries known in the art. In some embodiments, a FANA-ASO includes an internucleoside linkage including a phosphate, thereby being an oligonucleotide. In some embodiments, the sugar-modified nucleosides and/or 2′-deoxynucleosides include a phosphate, thereby being sugar-modified nucleotides and/or 2′-deoxynucleotides. In some embodiments, a FANA-ASO includes an internucleoside linkage including a phosphorothioate. In some embodiments, the internucleoside linkage is selected from phosphorothioate, phosphorodithioate, methylphosphorothioate, Rp-phosphorothioate, Sp-phosphorothioate. In some embodiments, the a FANA-ASO includes one or more internucleotide linkages selected from: (a) phosphodiester; (b) phosphotriester; (c) phosphorothioate; (d) phosphorodithioate; (e) Rp-phosphorothioate; (f) Sp-phosphorothioate; (g) boranophosphate; (h) methylene (methylimino) (3′CH₂—N(CH₃)—O5′); (i) 3′-thioformacetal (3′S—CH₂—O5′); (j) amide (3′CH₂—C(O)NH—O5′); (k) methylphosphonate; (l) phosphoramidate (3′-OP(O₂)—N5′); and (m) any combination of (a) to (1).

In certain embodiments, the FANA-ASOs can include 2′FANA modified nucleotides at any position within the oligonucleotide. In some embodiments, FANA-ASOs including alternating segments or units of sugar-modified nucleotides (e.g., arabinonucleotide analogues [e.g., 2′F-ANA]) and 2′-deoxyribonucleotides (DNA) are utilized. In some embodiments, a FANA-ASO disclosed herein includes at least 2 of each of sugar-modified nucleotide and 2′-deoxynucleotide segments, thereby having at least 4 alternating segments overall. Each alternating segment or unit may independently contain 1 or a plurality of nucleotides. In some embodiments, each alternating segment or unit may independently contain 1 or 2 nucleotides. In some embodiments, the segments each include 1 nucleotide. In some embodiments, the segments each include 2 nucleotides. In some embodiments, the plurality of nucleotides may consist of 2, 3, 4, 5 or 6 nucleotides. A FANA-ASO may contain an odd or even number of alternating segments or units and may commence and/or terminate with a segment containing sugar-modified nucleotide residues or DNA residues. Thus, a FANA-ASO may be represented as follows:

A₁-D₁-A₂-D₂-A₃-D₃ . . . A_{z}-D_{z}

Where each of A₁, A₂, etc. represents a unit of one or more (e.g., 1 or 2) sugar-modified nucleotide residues (e.g., 2′F-ANA) and each of D₁, D₂, etc. represents a unit of one or more (e.g., 1 or 2) DNA residues. The number of residues within each unit may be the same or variable from one unit to another. The oligonucleotide may have an odd or an even number of units. The oligonucleotide may start (i.e. at its 5′ end) with either a sugar-modified nucleotide-containing unit (e.g., a 2′F-ANA-containing unit) or a DNA-containing unit. The oligonucleotide may terminate (i.e. at its 3′ end) with either a sugar-modified nucleotide-containing unit or a DNA-containing unit. The total number of units may be as few as 4 (i.e. at least 2 of each type).

In some embodiments, a FANA-ASO disclosed herein includes alternating segments or units of arabinonucleotides and 2′-deoxynucleotides, wherein the segments or units each independently include at least one arabinonucleotide or 2′-deoxynucleotide, respectively. In some embodiments, the segments each independently include 1 to 2 arabinonucleotides or 2′-deoxynucleotides. In some embodiments, the segments each independently include 2 to 5 or 3 to 4 arabinonucleotides or 2′-deoxynucleotides. In some embodiments, a FANA-ASO disclosed herein includes alternating segments or units of arabinonucleotides and 2′-deoxynucleotides, wherein the segments or units each include one arabinonucleotide or 2′-deoxynucleotide, respectively. In some embodiments, the segments each independently include about 3 arabinonucleotides or 2′-deoxynucleotides. In some embodiments, a FANA-ASO disclosed herein includes alternating segments or units of arabinonucleotides and 2′-deoxynucleotides, wherein the segments or units each include one arabinonucleotide or 2′-deoxynucleotide, respectively. In some embodiments, a FANA-ASO disclosed herein includes alternating segments or units of arabinonucleotides and 2′-deoxynucleotides, wherein said segments or units each include two arabinonucleotides or 2′-deoxynucleotides, respectively.

In some embodiments, a FANA-ASO disclosed herein has a structure selected from:
a) (Aₓ-D_{y})ₙ I
b) (D_{y}-Aₓ)ₙ II
c) (Aₓ-D_{y})ₘ-Aₓ-D_{y}-Aₓ III
d) (D_{y}-Aₓ)ₘ-D_{y}-Aₓ-D_{y} IV
   wherein each of m, x and y are each independently an integer greater than or equal to 1, n is an integer greater than or equal to 2, A is a sugar-modified nucleotide and D is a 2′-deoxyribonucleotide. For example, a FANA-ASO disclosed herein has structure I wherein x=1, y=1 and n=10, thereby having a structure:

A-D-A-D-A-D-A-D-A-D-A-D-A-D-A-D-A-D-A-D.

In another example, a FANA-ASO disclosed herein has structure II wherein x=1, y=1 and n=10, thereby having a structure:

D-A-D-A-D-A-D-A-D-A-D-A-D-A-D-A-D-A-D-A.

In another example, a FANA-ASO disclosed herein has structure III wherein x=1, y=1 and n=9, thereby having a structure:

A-D-A-D-A-D-A-D-A-D-A-D-A-D-A-D-A-D-A-D-A.

In another example, a FANA-ASO disclosed herein has structure IV wherein x=1, y=1 and n=9, thereby having a structure:

D-A-D-A-D-A-D-A-D-A-D-A-D-A-D-A-D-A-D-A-D.

In another example, a FANA-ASO disclosed herein has structure I wherein x=2, y=2 and n=5, thereby having a structure:

A-A-D-D-A-A-D-D-A-A-D-D-A-A-D-D-A-A-D-D.

In another example, a FANA-ASO disclosed herein has structure II wherein x=2, y=2 and n=5, thereby having a structure:

D-D-A-A-D-D-A-A-D-D-A-A-D-D-A-A-D-D-A-A.

In another example, a FANA-ASO disclosed herein has structure III wherein x=2, y=2 and m=4, thereby having a structure:

A-A-D-D-A-A-D-D-A-A-D-D-A-A-D-D-A-A-D-D-A-A.

In another example, a FANA-ASO disclosed herein has structure IV wherein x=2, y=2 and m=4, thereby having a structure:

D-D-A-A-D-D-A-A-D-D-A-A-D-D-A-A-D-D-A-A-D-D.

The formulas shown in Table 1 may be applied to any sequence, or a portion thereof, wherein X represents a nucleotide (A, C, G, T, or U), and wherein bold and underlined nucleotides represent sugar-modified or 2′F-ANA-modified nucleotide with backbone phosphorothioate linkages.

TABLE 1
Exemplary 2′FANA nucleoside
placement within 21-mer FANA-ASOs

| 21 nucleotides | Formula |
|---|---|
| Formula 1 | XXXXXXXXXXXXXXXXXXXXX |
| Formula 2 | XXXXXXXXXXXXXXXXXXXXX |
| Formula 3 | XXXXXXXXXXXXXXXXXXXXX |
| Formula 4 | XXXXXXXXXXXXXXXXXXXXX |
| Formula 5 | XXXXXXXXXXXXXXXXXXXXX |
| Formula 6 | XXXXXXXXXXXXXXXXXXXXX |
| Formula 7 | XXXXXXXXXXXXXXXXXXXXX |
| Formula 8 | XXXXXXXXXXXXXXXXXXXXX |
| Formula 9 | XXXXXXXXXXXXXXXXXXXXX |
| Formula 10 | XXXXXXXXXXXXXXXXXXXXX |
| Formula 11 | XXXXXXXXXXXXXXXXXXXXX |
| Formula 12 | XXXXXXXXXXXXXXXXXXXXX |
| Formula 13 | XXXXXXXXXXXXXXXXXXXXX |
| Formula 14 | XXXXXXXXXXXXXXXXXXXXX |
| Formula 15 | XXXXXXXXXXXXXXXXXXXXX |
| Formula 16 | XXXXXXXXXXXXXXXXXXXXX |

Specific examples of FANA-ASO molecules and sequences are shown in SEQ ID NOs: 1-536 in Table 2.

TABLE 2

| SEQ ID NO | Name | Sequence |
|---|---|---|
| SEQ ID NO: 1 | AUM-A34-001 | ACACTGTCGTCGAATGGCCACT |
| SEQ ID NO: 2 | AUM-A34-002 | CACACTGTCGTCGAATGGCCAC |
| SEQ ID NO: 3 | AUM-A34-003 | ATACATCCATGGCTAATGAATT |
| SEQ ID NO: 4 | AUM-A34-004 | AATACATCCATGGCTAATGAAT |
| SEQ ID NO: 5 | AUM-A34-005 | TGTCTTTCCTGCTGCTTCTGCC |
| SEQ ID NO: 6 | AUM-A34-006 | TTGTCTTTCCTGCTGCTTCTGC |
| SEQ ID NO: 7 | AUM-A34-007 | TCTTCTCAGCCACTGTTGCCAC |
| SEQ ID NO: 8 | AUM-A34-008 | GGTCTTCTCAGCCACTGTTGCC |
| SEQ ID NO: 9 | AUM-A34-009 | GCTCTTTGGTCTTCTCAGCCAC |
| SEQ ID NO: 10 | AUM-A34-010 | TCACTTGCTCTTTGGTCTTCTC |
| SEQ ID NO: 11 | AUM-A34-011 | TTTGTCACTTGCTCTTTGGTCT |
| SEQ ID NO: 12 | AUM-A34-012 | TGTCTTCTGGGCTACTGCTGTC |
| SEQ ID NO: 13 | AUM-A34-013 | CCAGTGGCTGCTGCAATGCTCC |
| SEQ ID NO: 14 | AUM-A34-014 | AGAATTCCTTCCTGTGGGGCTC |
| SEQ ID NO: 15 | AUM-A34-015 | ATATCTTCCAGAATTCCTTCCT |
| SEQ ID NO: 16 | AUM-A34-016 | CATATCTTCCAGAATTCCTTCC |
| SEQ ID NO: 17 | AUM-A34-017 | GCATATCTTCCAGAATTCCTTC |
| SEQ ID NO: 18 | AUM-A34-018 | AGGATCCACAGGCATATCTTCC |
| SEQ ID NO: 19 | AUM-A34-019 | CTCATTGTCAGGATCCACAGGC |
| SEQ ID NO: 20 | AUM-A34-020 | CATAAGCCTCATTGTCAGGATC |
| SEQ ID NO: 21 | AUM-A34-021 | TCATAAGCCTCATTGTCAGGAT |
| SEQ ID NO: 22 | AUM-A34-022 | CTCAGAAGGCATTTCATAAGCC |
| SEQ ID NO: 23 | AUM-A34-023 | CGTAGTCTTGATACCCTTCCTC |
| SEQ ID NO: 24 | AUM-A34-024 | TCGTAGTCTTGATACCCTTCCT |
| SEQ ID NO: 25 | AUM-A34-025 | CAGGTTCGTAGTCTTGATACCC |
| SEQ ID NO: 26 | AUM-A34-026 | ATTTCTTAGGCTTCAGGTTCGT |
| SEQ ID NO: 27 | AUM-A34-027 | ATATTTCTTAGGCTTCAGGTTC |
| SEQ ID NO: 28 | AUM-A34-028 | AGATATTTCTTAGGCTTCAGGT |
| SEQ ID NO: 29 | AUM-A34-029 | CAGATCTCAAGAAACTGGGAGC |
| SEQ ID NO: 30 | AUM-A34-030 | CTGTCAGCAGATCTCAAGAAAC |
| SEQ ID NO: 31 | AUM-A34-031 | TCATGACTGGGCACATTGGAAC |
| SEQ ID NO: 32 | AUM-A34-032 | GTACAGATACTTCAATCACTGC |
| SEQ ID NO: 33 | AUM-A34-033 | AGTGAAAGGGAAGCACCGAAAT |
| SEQ ID NO: 34 | AUM-A34-034 | TAACATCGTAGATTGAAGCCAC |
| SEQ ID NO: 35 | AUM-A34-035 | TTTAACATCGTAGATTGAAGCC |
| SEQ ID NO: 36 | AUM-A34-036 | TCAGTTCTTAATTCATGTTGCT |
| SEQ ID NO: 37 | AUM-A34-037 | GTCAGTTCTTAATTCATGTTGC |
| SEQ ID NO: 38 | AUM-A34-038 | ACTTAAGGAACCAGTGCATACC |
| SEQ ID NO: 39 | AUM-A34-039 | AATCACAGCCACTTAAGGAACC |
| SEQ ID NO: 40 | AUM-A34-040 | TAATCACAGCCACTTAAGGAAC |
| SEQ ID NO: 41 | AUM-A34-041 | TCAATAATTAATCACAGCCACT |
| SEQ ID NO: 42 | AUM-A34-042 | TTCAATAATTAATCACAGCCAC |
| SEQ ID NO: 43 | AUM-A34-043 | CTTTCAATAATTAATCACAGCC |
| SEQ ID NO: 44 | AUM-A34-044 | TACAATAGTAGTTGGGGTCTTC |
| SEQ ID NO: 45 | AUM-A34-045 | ATTGAAGGGAGAAATAGACCAC |
| SEQ ID NO: 46 | AUM-A34-046 | TGACAGGATTGAAGGGAGAAAT |
| SEQ ID NO: 47 | AUM-A34-047 | GTCAGAAAGGTACAGCATTCAC |
| SEQ ID NO: 48 | AUM-A34-048 | TTGTCAGAAAGGTACAGCATTC |
| SEQ ID NO: 49 | AUM-A34-049 | ATTGTCAGAAAGGTACAGCATT |
| SEQ ID NO: 50 | AUM-A34-050 | TATTGTCAGAAAGGTACAGCAT |
| SEQ ID NO: 51 | AUM-A34-051 | TCTTCTACACTGCTTAGTTCCC |
| SEQ ID NO: 52 | AUM-A34-052 | TGACTCTGGTAGTTCCAACGAT |
| SEQ ID NO: 53 | AUM-A34-053 | AGGTGACTCTGGTAGTTCCAAC |
| SEQ ID NO: 54 | AUM-A34-054 | TTAAGGTGACTCTGGTAGTTCC |
| SEQ ID NO: 55 | AUM-A34-055 | TTTAAAGGAGGCCATGAAATTT |
| SEQ ID NO: 56 | AUM-A34-056 | TCCTAGAATTCATATATTTGGC |
| SEQ ID NO: 57 | AUM-A34-057 | TGAATACATATAAACTGCTAGC |
| SEQ ID NO: 58 | AUM-A34-058 | CTCATGAATACATATAAACTGC |
| SEQ ID NO: 59 | AUM-A34-059 | ACTCATTCCTCCTTCCTTCCTC |
| SEQ ID NO: 60 | AUM-A34-060 | TCACTCATTCCTCCTTCCTTCC |
| SEQ ID NO: 61 | AUM-A34-061 | GTCACTCATTCCTCCTTCCTTC |
| SEQ ID NO: 62 | AUM-A34-062 | CACTCTGTAGTAGTCTCTCTTC |
| SEQ ID NO: 63 | AUM-A34-063 | CTTAGCACTCTGTAGTAGTCTC |
| SEQ ID NO: 64 | AUM-A34-064 | ACTTACCATTTCTCTCTAGTGT |
| SEQ ID NO: 65 | AUM-A34-065 | TCCTAACCGCCACTTTCTAACC |
| SEQ ID NO: 66 | AUM-A34-066 | ATATATCCTAACCGCCACTTTC |
| SEQ ID NO: 67 | AUM-A34-067 | AATATATCCTAACCGCCACTTT |
| SEQ ID NO: 68 | AUM-A34-068 | AAATATATCCTAACCGCCACTT |
| SEQ ID NO: 69 | AUM-A34-069 | AATATGCTGCTTTAGGTAGATT |
| SEQ ID NO: 70 | AUM-A34-070 | TCTAGTTCTGTCCTCTATTTCT |
| SEQ ID NO: 71 | AUM-A34-071 | GTCTAGTTCTGTCCTCTATTTC |
| SEQ ID NO: 72 | AUM-A34-072 | AGTCTAGTTCTGTCCTCTATTT |
| SEQ ID NO: 73 | AUM-A34-073 | TATCAGTCTAGTTCTGTCCTCT |
| SEQ ID NO: 74 | AUM-A34-074 | CTATCAGTCTAGTTCTGTCCTC |
| SEQ ID NO: 75 | AUM-A34-075 | TGCTATCAGTCTAGTTCTGTCC |
| SEQ ID NO: 76 | AUM-A34-076 | AATTGTTCTAGGTCACTGCTAT |
| SEQ ID NO: 77 | AUM-A34-077 | AGTGAATATGAGACAAGCTTCC |
| SEQ ID NO: 78 | AUM-A34-078 | GAGTGAATATGAGACAAGCTTC |
| SEQ ID NO: 79 | AUM-A34-079 | CACATTAGATTGTTCTGTTCCC |
| SEQ ID NO: 80 | AUM-A34-080 | ATAGCTACATACTGGATAAGCC |
| SEQ ID NO: 81 | AUM-A34-081 | AATAGCTACATACTGGATAAGC |
| SEQ ID NO: 82 | AUM-A34-082 | ACACTGTCGTCGAATGGCCAC |
| SEQ ID NO: 83 | AUM-A34-083 | GCTAATGAATTCCTTTACACC |
| SEQ ID NO: 84 | AUM-A34-084 | ATACATCCATGGCTAATGAAT |
| SEQ ID NO: 85 | AUM-A34-085 | ACAACTCCCTCCTTGGCCTTT |
| SEQ ID NO: 86 | AUM-A34-086 | GTCTTTCCTGCTGCTTCTGCC |
| SEQ ID NO: 87 | AUM-A34-087 | TGTCTTTCCTGCTGCTTCTGC |
| SEQ ID NO: 88 | AUM-A34-088 | CTTCTCAGCCACTGTTGCCAC |
| SEQ ID NO: 89 | AUM-A34-089 | GTCTTCTCAGCCACTGTTGCC |
| SEQ ID NO: 90 | AUM-A34-090 | GGTCTTCTCAGCCACTGTTGC |
| SEQ ID NO: 91 | AUM-A34-091 | CTCTTTGGTCTTCTCAGCCAC |
| SEQ ID NO: 92 | AUM-A34-092 | TCACTTGCTCTTTGGTCTTCT |
| SEQ ID NO: 93 | AUM-A34-093 | GTCACTTGCTCTTTGGTCTTC |
| SEQ ID NO: 94 | AUM-A34-094 | TTGTCACTTGCTCTTTGGTCT |
| SEQ ID NO: 95 | AUM-A34-095 | TTTGTCACTTGCTCTTTGGTC |
| SEQ ID NO: 96 | AUM-A34-096 | AACATTTGTCACTTGCTCTTT |
| SEQ ID NO: 97 | AUM-A34-097 | GTCTTCTGGGCTACTGCTGTC |
| SEQ ID NO: 98 | AUM-A34-098 | TGTCTTCTGGGCTACTGCTGT |
| SEQ ID NO: 99 | AUM-A34-099 | ACTGTCTTCTGGGCTACTGCT |
| SEQ ID NO: 100 | AUM-A34-100 | CCAGTGGCTGCTGCAATGCTC |
| SEQ ID NO: 101 | AUM-A34-101 | AGAATTCCTTCCTGTGGGGCT |
| SEQ ID NO: 102 | AUM-A34-102 | CAGAATTCCTTCCTGTGGGGC |
| SEQ ID NO: 103 | AUM-A34-103 | TATCTTCCAGAATTCCTTCCT |
| SEQ ID NO: 104 | AUM-A34-104 | ATATCTTCCAGAATTCCTTCC |
| SEQ ID NO: 105 | AUM-A34-105 | CATATCTTCCAGAATTCCTTC |
| SEQ ID NO: 106 | AUM-A34-106 | ATTGTCAGGATCCACAGGCAT |
| SEQ ID NO: 107 | AUM-A34-107 | TCATTGTCAGGATCCACAGGC |
| SEQ ID NO: 108 | AUM-A34-108 | TCAGAAGGCATTTCATAAGCC |
| SEQ ID NO: 109 | AUM-A34-109 | CTCAGAAGGCATTTCATAAGC |
| SEQ ID NO: 110 | AUM-A34-110 | GTAGTCTTGATACCCTTCCTC |
| SEQ ID NO: 111 | AUM-A34-111 | TCGTAGTCTTGATACCCTTCC |
| SEQ ID NO: 112 | AUM-A34-112 | TTTCTTAGGCTTCAGGTTCGT |
| SEQ ID NO: 113 | AUM-A34-113 | TATTTCTTAGGCTTCAGGTTC |
| SEQ ID NO: 114 | AUM-A34-114 | ATATTTCTTAGGCTTCAGGTT |
| SEQ ID NO: 115 | AUM-A34-115 | AGATCTCAAGAAACTGGGAGC |
| SEQ ID NO: 116 | AUM-A34-116 | AGCACTTGTACAGGATGGAAC |
| SEQ ID NO: 117 | AUM-A34-117 | AACTGAGCACTTGTACAGGAT |
| SEQ ID NO: 118 | AUM-A34-118 | ATGACTGGGCACATTGGAACT |
| SEQ ID NO: 119 | AUM-A34-119 | CATGACTGGGCACATTGGAAC |
| SEQ ID NO: 120 | AUM-A34-120 | ACAGATACTTCAATCACTGCT |
| SEQ ID NO: 121 | AUM-A34-121 | TTAACATCGTAGATTGAAGCC |
| SEQ ID NO: 122 | AUM-A34-122 | TTTAACATCGTAGATTGAAGC |
| SEQ ID NO: 123 | AUM-A34-123 | TTAGAAATAAGTGGTAGTCAC |
| SEQ ID NO: 124 | AUM-A34-124 | TCAGTTCTTAATTCATGTTGC |
| SEQ ID NO: 125 | AUM-A34-125 | AGTGTAGGGTTAATGTTCCAT |
| SEQ ID NO: 126 | AUM-A34-126 | CAGTGTTGCTTCAGGGAATTC |
| SEQ ID NO: 127 | AUM-A34-127 | CTTAAGGAACCAGTGCATACC |
| SEQ ID NO: 128 | AUM-A34-128 | ATCACAGCCACTTAAGGAACC |
| SEQ ID NO: 129 | AUM-A34-129 | AATCACAGCCACTTAAGGAAC |
| SEQ ID NO: 130 | AUM-A34-130 | TCAATAATTAATCACAGCCAC |
| SEQ ID NO: 131 | AUM-A34-131 | TACAATAGTAGTTGGGGTCTT |
| SEQ ID NO: 132 | AUM-A34-132 | GATTGAAGGGAGAAATAGACC |
| SEQ ID NO: 133 | AUM-A34-133 | TCAGAAAGGTACAGCATTCAC |
| SEQ ID NO: 134 | AUM-A34-134 | TGTCAGAAAGGTACAGCATTC |
| SEQ ID NO: 135 | AUM-A34-135 | TTGTCAGAAAGGTACAGCATT |
| SEQ ID NO: 136 | AUM-A34-136 | ATTGTCAGAAAGGTACAGCAT |
| SEQ ID NO: 137 | AUM-A34-137 | CTTCTACACTGCTTAGTTCCC |
| SEQ ID NO: 138 | AUM-A34-138 | TCTTCTACACTGCTTAGTTCC |
| SEQ ID NO: 139 | AUM-A34-139 | AAATCATCTTCTACACTGCTT |
| SEQ ID NO: 140 | AUM-A34-140 | TTGAATGTGCTAATATGTGCT |
| SEQ ID NO: 141 | AUM-A34-141 | CTTGAATGTGCTAATATGTGC |
| SEQ ID NO: 142 | AUM-A34-142 | TCTCAGAGCCTTGAATGTGCT |
| SEQ ID NO: 143 | AUM-A34-143 | ATGTTTAAAGGCATTTCCTGT |
| SEQ ID NO: 144 | AUM-A34-144 | GGTGACTCTGGTAGTTCCAAC |
| SEQ ID NO: 145 | AUM-A34-145 | TAAGGTGACTCTGGTAGTTCC |
| SEQ ID NO: 146 | AUM-A34-146 | TTAAGGTGACTCTGGTAGTTC |
| SEQ ID NO: 147 | AUM-A34-147 | TTTAAAGGAGGCCATGAAATT |
| SEQ ID NO: 148 | AUM-A34-148 | CCTAGAATTCATATATTTGGC |
| SEQ ID NO: 149 | AUM-A34-149 | CTCATTCCTCCTTCCTTCCTC |
| SEQ ID NO: 150 | AUM-A34-150 | ACTCATTCCTCCTTCCTTCCT |
| SEQ ID NO: 151 | AUM-A34-151 | CACTCATTCCTCCTTCCTTCC |
| SEQ ID NO: 152 | AUM-A34-152 | TCACTCATTCCTCCTTCCTTC |
| SEQ ID NO: 153 | AUM-A34-153 | ACTCTGTAGTAGTCTCTCTTC |
| SEQ ID NO: 154 | AUM-A34-154 | ATATCCTAACCGCCACTTTCT |
| SEQ ID NO: 155 | AUM-A34-155 | ATATATCCTAACCGCCACTTT |
| SEQ ID NO: 156 | AUM-A34-156 | AATATATCCTAACCGCCACTT |
| SEQ ID NO: 157 | AUM-A34-157 | AAATATATCCTAACCGCCACT |
| SEQ ID NO: 158 | AUM-A34-158 | TAGTTCTGTCCTCTATTTCTT |
| SEQ ID NO: 159 | AUM-A34-159 | TCTAGTTCTGTCCTCTATTTC |
| SEQ ID NO: 160 | AUM-A34-160 | TATCAGTCTAGTTCTGTCCTC |
| SEQ ID NO: 161 | AUM-A34-161 | GCTATCAGTCTAGTTCTGTCC |
| SEQ ID NO: 162 | AUM-A34-162 | ATTGTTCTAGGTCACTGCTAT |
| SEQ ID NO: 163 | AUM-A34-163 | AAATTGTTCTAGGTCACTGCT |
| SEQ ID NO: 164 | AUM-A34-164 | TTTGTTTAAGTGTTTGGTCCC |
| SEQ ID NO: 165 | AUM-A34-165 | TGAATATGAGACAAGCTTCCT |
| SEQ ID NO: 166 | AUM-A34-166 | GTGAATATGAGACAAGCTTCC |
| SEQ ID NO: 167 | AUM-A34-167 | AGTGAATATGAGACAAGCTTC |
| SEQ ID NO: 168 | AUM-A34-168 | AAATAAACCTAGGACTGGATT |
| SEQ ID NO: 169 | AUM-A34-169 | TGGTAAAGCCGACCGTGGAGT |
| SEQ ID NO: 170 | AUM-A34-170 | ACATTAGATTGTTCTGTTCCC |
| SEQ ID NO: 171 | AUM-A34-171 | CACATTAGATTGTTCTGTTCC |
| SEQ ID NO: 172 | AUM-A34-172 | TAGCTACATACTGGATAAGCC |
| SEQ ID NO: 173 | AUM-A34-173 | ATAGCTACATACTGGATAAGC |
| SEQ ID NO: 174 | AUM-A34-174 | AGTTCTCCGCTCACGAGGGT |
| SEQ ID NO: 175 | AUM-A34-175 | CCACTCCCAGTTCTCCGCTC |
| SEQ ID NO: 176 | AUM-A34-176 | CACTGTCGTCGAATGGCCAC |
| SEQ ID NO: 177 | AUM-A34-177 | CACACTGTCGTCGAATGGCC |
| SEQ ID NO: 178 | AUM-A34-178 | CTAATGAATTCCTTTACACC |
| SEQ ID NO: 179 | AUM-A34-179 | ACAACTCCCTCCTTGGCCTT |
| SEQ ID NO: 180 | AUM-A34-180 | GTCTTTCCTGCTGCTTCTGC |
| SEQ ID NO: 181 | AUM-A34-181 | TTGTCTTTCCTGCTGCTTCT |
| SEQ ID NO: 182 | AUM-A34-182 | TTTGTCTTTCCTGCTGCTTC |
| SEQ ID NO: 183 | AUM-A34-183 | TTCTCAGCCACTGTTGCCAC |
| SEQ ID NO: 184 | AUM-A34-184 | TCTTCTCAGCCACTGTTGCC |
| SEQ ID NO: 185 | AUM-A34-185 | GTCTTCTCAGCCACTGTTGC |
| SEQ ID NO: 186 | AUM-A34-186 | GCTCTTTGGTCTTCTCAGCC |
| SEQ ID NO: 187 | AUM-A34-187 | TCACTTGCTCTTTGGTCTTC |
| SEQ ID NO: 188 | AUM-A34-188 | TGTCACTTGCTCTTTGGTCT |
| SEQ ID NO: 189 | AUM-A34-189 | TTGTCACTTGCTCTTTGGTC |
| SEQ ID NO: 190 | AUM-A34-190 | TTTGTCACTTGCTCTTTGGT |
| SEQ ID NO: 191 | AUM-A34-191 | ACATTTGTCACTTGCTCTTT |
| SEQ ID NO: 192 | AUM-A34-192 | AACATTTGTCACTTGCTCTT |
| SEQ ID NO: 193 | AUM-A34-193 | ACTGTCTTCTGGGCTACTGC |
| SEQ ID NO: 194 | AUM-A34-194 | AGAATTCCTTCCTGTGGGGC |
| SEQ ID NO: 195 | AUM-A34-195 | TATCTTCCAGAATTCCTTCC |
| SEQ ID NO: 196 | AUM-A34-196 | ATATCTTCCAGAATTCCTTC |
| SEQ ID NO: 197 | AUM-A34-197 | TTGTCAGGATCCACAGGCAT |
| SEQ ID NO: 198 | AUM-A34-198 | CATTGTCAGGATCCACAGGC |
| SEQ ID NO: 199 | AUM-A34-199 | TCAGAAGGCATTTCATAAGC |
| SEQ ID NO: 200 | AUM-A34-200 | TAGTCTTGATACCCTTCCTC |
| SEQ ID NO: 201 | AUM-A34-201 | CGTAGTCTTGATACCCTTCC |
| SEQ ID NO: 202 | AUM-A34-202 | TCGTAGTCTTGATACCCTTC |
| SEQ ID NO: 203 | AUM-A34-203 | TTCTTAGGCTTCAGGTTCGT |
| SEQ ID NO: 204 | AUM-A34-204 | ATTTCTTAGGCTTCAGGTTC |
| SEQ ID NO: 205 | AUM-A34-205 | TATTTCTTAGGCTTCAGGTT |
| SEQ ID NO: 206 | AUM-A34-206 | ATATTTCTTAGGCTTCAGGT |
| SEQ ID NO: 207 | AUM-A34-207 | GATCTCAAGAAACTGGGAGC |
| SEQ ID NO: 208 | AUM-A34-208 | TGACTGGGCACATTGGAACT |
| SEQ ID NO: 209 | AUM-A34-209 | ATGACTGGGCACATTGGAAC |
| SEQ ID NO: 210 | AUM-A34-210 | TCACTGCTGATGGAAGACTT |
| SEQ ID NO: 211 | AUM-A34-211 | ACAGATACTTCAATCACTGC |
| SEQ ID NO: 212 | AUM-A34-212 | TAACATCGTAGATTGAAGCC |
| SEQ ID NO: 213 | AUM-A34-213 | TTAACATCGTAGATTGAAGC |
| SEQ ID NO: 214 | AUM-A34-214 | TAGAAATAAGTGGTAGTCAC |
| SEQ ID NO: 215 | AUM-A34-215 | TAGTTTCATGCTCACATATT |
| SEQ ID NO: 216 | AUM-A34-216 | ATAGTTTCATGCTCACATAT |
| SEQ ID NO: 217 | AUM-A34-217 | TTATAGGTGCATAGTTTCAT |
| SEQ ID NO: 218 | AUM-A34-218 | ATTTATAGGTGCATAGTTTC |
| SEQ ID NO: 219 | AUM-A34-219 | AGTATTTATAGGTGCATAGT |
| SEQ ID NO: 220 | AUM-A34-220 | TTATTAAAGTGAGATGGGAT |
| SEQ ID NO: 221 | AUM-A34-221 | AGTTCTTAATTCATGTTGCT |
| SEQ ID NO: 222 | AUM-A34-222 | CAGTTCTTAATTCATGTTGC |
| SEQ ID NO: 223 | AUM-A34-223 | GAGTGTAGGGTTAATGTTCC |
| SEQ ID NO: 224 | AUM-A34-224 | AGTGTTGCTTCAGGGAATTC |
| SEQ ID NO: 225 | AUM-A34-225 | ACTTCTGGCAGTGTTGCTTC |
| SEQ ID NO: 226 | AUM-A34-226 | TCACAGCCACTTAAGGAACC |
| SEQ ID NO: 227 | AUM-A34-227 | ATCACAGCCACTTAAGGAAC |
| SEQ ID NO: 228 | AUM-A34-228 | ATAATTAATCACAGCCACTT |
| SEQ ID NO: 229 | AUM-A34-229 | AATAATTAATCACAGCCACT |
| SEQ ID NO: 230 | AUM-A34-230 | CAATAATTAATCACAGCCAC |
| SEQ ID NO: 231 | AUM-A34-231 | TTCAATAATTAATCACAGCC |
| SEQ ID NO: 232 | AUM-A34-232 | TACAATAGTAGTTGGGGTCT |
| SEQ ID NO: 233 | AUM-A34-233 | ATTGAAGGGAGAAATAGACC |
| SEQ ID NO: 234 | AUM-A34-234 | GTCAGAAAGGTACAGCATTC |
| SEQ ID NO: 235 | AUM-A34-235 | TGTCAGAAAGGTACAGCATT |
| SEQ ID NO: 236 | AUM-A34-236 | TTGTCAGAAAGGTACAGCAT |
| SEQ ID NO: 237 | AUM-A34-237 | TATTGTCAGAAAGGTACAGC |
| SEQ ID NO: 238 | AUM-A34-238 | ATTTATTGTCAGAAAGGTAC |
| SEQ ID NO: 239 | AUM-A34-239 | TTCTACACTGCTTAGTTCCC |
| SEQ ID NO: 240 | AUM-A34-240 | CTTCTACACTGCTTAGTTCC |
| SEQ ID NO: 241 | AUM-A34-241 | AATCATCTTCTACACTGCTT |
| SEQ ID NO: 242 | AUM-A34-242 | AAATCATCTTCTACACTGCT |
| SEQ ID NO: 243 | AUM-A34-243 | TGAATGTGCTAATATGTGCT |
| SEQ ID NO: 244 | AUM-A34-244 | TTGAATGTGCTAATATGTGC |
| SEQ ID NO: 245 | AUM-A34-245 | TCTCAGAGCCTTGAATGTGC |
| SEQ ID NO: 246 | AUM-A34-246 | TGTTTAAAGGCATTTCCTGT |
| SEQ ID NO: 247 | AUM-A34-247 | GTGACTCTGGTAGTTCCAAC |
| SEQ ID NO: 248 | AUM-A34-248 | AAGGTGACTCTGGTAGTTCC |
| SEQ ID NO: 249 | AUM-A34-249 | TTTAAAGGAGGCCATGAAAT |
| SEQ ID NO: 250 | AUM-A34-250 | GAATTCATATATTTGGCAAC |
| SEQ ID NO: 251 | AUM-A34-251 | CTAGAATTCATATATTTGGC |
| SEQ ID NO: 252 | AUM-A34-252 | TACTTTCTACTAGTGACTTT |
| SEQ ID NO: 253 | AUM-A34-253 | ATACTTTCTACTAGTGACTT |
| SEQ ID NO: 254 | AUM-A34-254 | TATACTTTCTACTAGTGACT |
| SEQ ID NO: 255 | AUM-A34-255 | TTATACTTTCTACTAGTGAC |
| SEQ ID NO: 256 | AUM-A34-256 | AATACATATAAACTGCTAGC |
| SEQ ID NO: 257 | AUM-A34-257 | CATGAATACATATAAACTGC |
| SEQ ID NO: 258 | AUM-A34-258 | ACTCATTCCTCCTTCCTTCC |
| SEQ ID NO: 259 | AUM-A34-259 | CACTCATTCCTCCTTCCTTC |
| SEQ ID NO: 260 | AUM-A34-260 | TCACTCATTCCTCCTTCCTT |
| SEQ ID NO: 261 | AUM-A34-261 | TAGTCTCTCTTCAATTAGGT |
| SEQ ID NO: 262 | AUM-A34-262 | ACTCTGTAGTAGTCTCTCTT |
| SEQ ID NO: 263 | AUM-A34-263 | ATATCCTAACCGCCACTTTC |
| SEQ ID NO: 264 | AUM-A34-264 | ATATATCCTAACCGCCACTT |
| SEQ ID NO: 265 | AUM-A34-265 | AATATATCCTAACCGCCACT |
| SEQ ID NO: 266 | AUM-A34-266 | AAATATATCCTAACCGCCAC |
| SEQ ID NO: 267 | AUM-A34-267 | AGTTCTGTCCTCTATTTCTT |
| SEQ ID NO: 268 | AUM-A34-268 | TAGTTCTGTCCTCTATTTCT |
| SEQ ID NO: 269 | AUM-A34-269 | CTAGTTCTGTCCTCTATTTC |
| SEQ ID NO: 270 | AUM-A34-270 | TCTAGTTCTGTCCTCTATTT |
| SEQ ID NO: 271 | AUM-A34-271 | TCAGTCTAGTTCTGTCCTCT |
| SEQ ID NO: 272 | AUM-A34-272 | TATCAGTCTAGTTCTGTCCT |
| SEQ ID NO: 273 | AUM-A34-273 | CTATCAGTCTAGTTCTGTCC |
| SEQ ID NO: 274 | AUM-A34-274 | TTGTTCTAGGTCACTGCTAT |
| SEQ ID NO: 275 | AUM-A34-275 | AATTGTTCTAGGTCACTGCT |
| SEQ ID NO: 276 | AUM-A34-276 | AAATTGTTCTAGGTCACTGC |
| SEQ ID NO: 277 | AUM-A34-277 | TTGTATCCACATAAATCCTT |
| SEQ ID NO: 278 | AUM-A34-278 | TTTGTATCCACATAAATCCT |
| SEQ ID NO: 279 | AUM-A34-279 | TATAAAGTAATTCACTGCTC |
| SEQ ID NO: 280 | AUM-A34-280 | TTGTTTAAGTGTTTGGTCCC |
| SEQ ID NO: 281 | AUM-A34-281 | TTTGTTTAAGTGTTTGGTCC |
| SEQ ID NO: 282 | AUM-A34-282 | TGAATATGAGACAAGCTTCC |
| SEQ ID NO: 283 | AUM-A34-283 | GTGAATATGAGACAAGCTTC |
| SEQ ID NO: 284 | AUM-A34-284 | AGTGAATATGAGACAAGCTT |
| SEQ ID NO: 285 | AUM-A34-285 | GGAGTGAATATGAGACAAGC |
| SEQ ID NO: 286 | AUM-A34-286 | TGAATGTCTCGGGAGTGAAT |
| SEQ ID NO: 287 | AUM-A34-287 | AATAAACCTAGGACTGGATT |
| SEQ ID NO: 288 | AUM-A34-288 | AAATAAACCTAGGACTGGAT |
| SEQ ID NO: 289 | AUM-A34-289 | CATTAGATTGTTCTGTTCCC |
| SEQ ID NO: 290 | AUM-A34-290 | ACATTAGATTGTTCTGTTCC |
| SEQ ID NO: 291 | AUM-A34-291 | CACATTAGATTGTTCTGTTC |
| SEQ ID NO: 292 | AUM-A34-292 | TAGCTACATACTGGATAAGC |
| SEQ ID NO: 293 | AUM-A34-293 | ACACTGTCGTCGAATGGCC |
| SEQ ID NO: 294 | AUM-A34-294 | CACACTGTCGTCGAATGGC |
| SEQ ID NO: 295 | AUM-A34-295 | TAATGAATTCCTTTACACC |
| SEQ ID NO: 296 | AUM-A34-296 | ACAACTCCCTCCTTGGCCT |
| SEQ ID NO: 297 | AUM-A34-297 | TGTCTTTCCTGCTGCTTCT |
| SEQ ID NO: 298 | AUM-A34-298 | TTGTCTTTCCTGCTGCTTC |
| SEQ ID NO: 299 | AUM-A34-299 | TTTGTCTTTCCTGCTGCTT |
| SEQ ID NO: 300 | AUM-A34-300 | CTTCTCAGCCACTGTTGCC |
| SEQ ID NO: 301 | AUM-A34-301 | TCTTCTCAGCCACTGTTGC |
| SEQ ID NO: 302 | AUM-A34-302 | CTCTTTGGTCTTCTCAGCC |
| SEQ ID NO: 303 | AUM-A34-303 | GCTCTTTGGTCTTCTCAGC |
| SEQ ID NO: 304 | AUM-A34-304 | TGTCACTTGCTCTTTGGTC |
| SEQ ID NO: 305 | AUM-A34-305 | TTGTCACTTGCTCTTTGGT |
| SEQ ID NO: 306 | AUM-A34-306 | ACATTTGTCACTTGCTCTT |
| SEQ ID NO: 307 | AUM-A34-307 | AACATTTGTCACTTGCTCT |
| SEQ ID NO: 308 | AUM-A34-308 | CAACATTTGTCACTTGCTC |
| SEQ ID NO: 309 | AUM-A34-309 | TCTTCTGGGCTACTGCTGT |
| SEQ ID NO: 310 | AUM-A34-310 | TGTCTTCTGGGCTACTGCT |
| SEQ ID NO: 311 | AUM-A34-311 | CTGTCTTCTGGGCTACTGC |
| SEQ ID NO: 312 | AUM-A34-312 | CCAGTGGCTGCTGCAATGC |
| SEQ ID NO: 313 | AUM-A34-313 | TATCTTCCAGAATTCCTTC |
| SEQ ID NO: 314 | AUM-A34-314 | ATATCTTCCAGAATTCCTT |
| SEQ ID NO: 315 | AUM-A34-315 | GCATATCTTCCAGAATTCC |
| SEQ ID NO: 316 | AUM-A34-316 | ATTGTCAGGATCCACAGGC |
| SEQ ID NO: 317 | AUM-A34-317 | TAGTCTTGATACCCTTCCT |
| SEQ ID NO: 318 | AUM-A34-318 | GTAGTCTTGATACCCTTCC |
| SEQ ID NO: 319 | AUM-A34-319 | CGTAGTCTTGATACCCTTC |
| SEQ ID NO: 320 | AUM-A34-320 | TCGTAGTCTTGATACCCTT |
| SEQ ID NO: 321 | AUM-A34-321 | TCTTAGGCTTCAGGTTCGT |
| SEQ ID NO: 322 | AUM-A34-322 | TTTCTTAGGCTTCAGGTTC |
| SEQ ID NO: 323 | AUM-A34-323 | ATTTCTTAGGCTTCAGGTT |
| SEQ ID NO: 324 | AUM-A34-324 | TATTTCTTAGGCTTCAGGT |
| SEQ ID NO: 325 | AUM-A34-325 | ATCTCAAGAAACTGGGAGC |
| SEQ ID NO: 326 | AUM-A34-326 | CACTTGTACAGGATGGAAC |
| SEQ ID NO: 327 | AUM-A34-327 | TGACTGGGCACATTGGAAC |
| SEQ ID NO: 328 | AUM-A34-328 | AGACTTCGAGATACACTGT |
| SEQ ID NO: 329 | AUM-A34-329 | AGATACTTCAATCACTGCT |
| SEQ ID NO: 330 | AUM-A34-330 | CAGATACTTCAATCACTGC |
| SEQ ID NO: 331 | AUM-A34-331 | GTACAGATACTTCAATCAC |
| SEQ ID NO: 332 | AUM-A34-332 | TCAGTGAAAGGGAAGCACC |
| SEQ ID NO: 333 | AUM-A34-333 | TAACATCGTAGATTGAAGC |
| SEQ ID NO: 334 | AUM-A34-334 | TAGTTTCATGCTCACATAT |
| SEQ ID NO: 335 | AUM-A34-335 | TATAGGTGCATAGTTTCAT |
| SEQ ID NO: 336 | AUM-A34-336 | TTTATAGGTGCATAGTTTC |
| SEQ ID NO: 337 | AUM-A34-337 | TATTAAAGTGAGATGGGAT |
| SEQ ID NO: 338 | AUM-A34-338 | AGTTCTTAATTCATGTTGC |
| SEQ ID NO: 339 | AUM-A34-339 | AGTGTAGGGTTAATGTTCC |
| SEQ ID NO: 340 | AUM-A34-340 | GAGTGTAGGGTTAATGTTC |
| SEQ ID NO: 341 | AUM-A34-341 | AGTGTTGCTTCAGGGAATT |
| SEQ ID NO: 342 | AUM-A34-342 | ACTTCTGGCAGTGTTGCTT |
| SEQ ID NO: 343 | AUM-A34-343 | ACTTAAGGAACCAGTGCAT |
| SEQ ID NO: 344 | AUM-A34-344 | TCACAGCCACTTAAGGAAC |
| SEQ ID NO: 345 | AUM-A34-345 | TAATTAATCACAGCCACTT |
| SEQ ID NO: 346 | AUM-A34-346 | ATAATTAATCACAGCCACT |
| SEQ ID NO: 347 | AUM-A34-347 | AATAATTAATCACAGCCAC |
| SEQ ID NO: 348 | AUM-A34-348 | TCAATAATTAATCACAGCC |
| SEQ ID NO: 349 | AUM-A34-349 | AATAGTAGTTGGGGTCTTC |
| SEQ ID NO: 350 | AUM-A34-350 | TACAATAGTAGTTGGGGTC |
| SEQ ID NO: 351 | AUM-A34-351 | TCAGAAAGGTACAGCATTC |
| SEQ ID NO: 352 | AUM-A34-352 | TGTCAGAAAGGTACAGCAT |
| SEQ ID NO: 353 | AUM-A34-353 | ATTGTCAGAAAGGTACAGC |
| SEQ ID NO: 354 | AUM-A34-354 | TCTACACTGCTTAGTTCCC |
| SEQ ID NO: 355 | AUM-A34-355 | TTCTACACTGCTTAGTTCC |
| SEQ ID NO: 356 | AUM-A34-356 | ATCATCTTCTACACTGCTT |
| SEQ ID NO: 357 | AUM-A34-357 | AATCATCTTCTACACTGCT |
| SEQ ID NO: 358 | AUM-A34-358 | AAATCATCTTCTACACTGC |
| SEQ ID NO: 359 | AUM-A34-359 | TGAATGTGCTAATATGTGC |
| SEQ ID NO: 360 | AUM-A34-360 | TCAGAGCCTTGAATGTGCT |
| SEQ ID NO: 361 | AUM-A34-361 | CTCAGAGCCTTGAATGTGC |
| SEQ ID NO: 362 | AUM-A34-362 | ATGTTTAAAGGCATTTCCT |
| SEQ ID NO: 363 | AUM-A34-363 | GATGTTTAAAGGCATTTCC |
| SEQ ID NO: 364 | AUM-A34-364 | TGACTCTGGTAGTTCCAAC |
| SEQ ID NO: 365 | AUM-A34-365 | AACATTTAAAGGAGGCCAT |
| SEQ ID NO: 366 | AUM-A34-366 | GGAGTTAATAGATCTTCCC |
| SEQ ID NO: 367 | AUM-A34-367 | TTTAGAAATGACTATGCCC |
| SEQ ID NO: 368 | AUM-A34-368 | TACTTTCTACTAGTGACTT |
| SEQ ID NO: 369 | AUM-A34-369 | ATACTTTCTACTAGTGACT |
| SEQ ID NO: 370 | AUM-A34-370 | TATACTTTCTACTAGTGAC |
| SEQ ID NO: 371 | AUM-A34-371 | CTCATTCCTCCTTCCTTCC |
| SEQ ID NO: 372 | AUM-A34-372 | ACTCATTCCTCCTTCCTTC |
| SEQ ID NO: 373 | AUM-A34-373 | TCACTCATTCCTCCTTCCT |
| SEQ ID NO: 374 | AUM-A34-374 | GTCACTCATTCCTCCTTCC |
| SEQ ID NO: 375 | AUM-A34-375 | AGTCTCTCTTCAATTAGGT |
| SEQ ID NO: 376 | AUM-A34-376 | ACTCTGTAGTAGTCTCTCT |
| SEQ ID NO: 377 | AUM-A34-377 | CACTCTGTAGTAGTCTCTC |
| SEQ ID NO: 378 | AUM-A34-378 | ATATCCTAACCGCCACTTT |
| SEQ ID NO: 379 | AUM-A34-379 | ATATATCCTAACCGCCACT |
| SEQ ID NO: 380 | AUM-A34-380 | AATATATCCTAACCGCCAC |
| SEQ ID NO: 381 | AUM-A34-381 | AGTTCTGTCCTCTATTTCT |
| SEQ ID NO: 382 | AUM-A34-382 | TAGTTCTGTCCTCTATTTC |
| SEQ ID NO: 383 | AUM-A34-383 | TCTAGTTCTGTCCTCTATT |
| SEQ ID NO: 384 | AUM-A34-384 | TCAGTCTAGTTCTGTCCTC |
| SEQ ID NO: 385 | AUM-A34-385 | TATCAGTCTAGTTCTGTCC |
| SEQ ID NO: 386 | AUM-A34-386 | CTATCAGTCTAGTTCTGTC |
| SEQ ID NO: 387 | AUM-A34-387 | TGTTCTAGGTCACTGCTAT |
| SEQ ID NO: 388 | AUM-A34-388 | ATTGTTCTAGGTCACTGCT |
| SEQ ID NO: 389 | AUM-A34-389 | AATTGTTCTAGGTCACTGC |
| SEQ ID NO: 390 | AUM-A34-390 | TGTATCCACATAAATCCTT |
| SEQ ID NO: 391 | AUM-A34-391 | TTGTATCCACATAAATCCT |
| SEQ ID NO: 392 | AUM-A34-392 | TTTGTATCCACATAAATCC |
| SEQ ID NO: 393 | AUM-A34-393 | ATAAAGTAATTCACTGCTC |
| SEQ ID NO: 394 | AUM-A34-394 | TTGTTTAAGTGTTTGGTCC |
| SEQ ID NO: 395 | AUM-A34-395 | TTTGTTTAAGTGTTTGGTC |
| SEQ ID NO: 396 | AUM-A34-396 | AATATGAGACAAGCTTCCT |
| SEQ ID NO: 397 | AUM-A34-397 | TGAATATGAGACAAGCTTC |
| SEQ ID NO: 398 | AUM-A34-398 | AGTGAATATGAGACAAGCT |
| SEQ ID NO: 399 | AUM-A34-399 | GAGTGAATATGAGACAAGC |
| SEQ ID NO: 400 | AUM-A34-400 | AATAAACCTAGGACTGGAT |
| SEQ ID NO: 401 | AUM-A34-401 | ATATGAGGCTGAATAACTT |
| SEQ ID NO: 402 | AUM-A34-402 | ATTAGATTGTTCTGTTCCC |
| SEQ ID NO: 403 | AUM-A34-403 | CATTAGATTGTTCTGTTCC |
| SEQ ID NO: 404 | AUM-A34-404 | ACATTAGATTGTTCTGTTC |
| SEQ ID NO: 405 | AUM-A34-405 | GCTACATACTGGATAAGCC |
| SEQ ID NO: 406 | AUM-A34-406 | AAATAGCTACATACTGGAT |
| SEQ ID NO: 407 | AUM-A34-407 | CCACTCCCAGTTCTCCGC |
| SEQ ID NO: 408 | AUM-A34-408 | CACTGTCGTCGAATGGCC |
| SEQ ID NO: 409 | AUM-A34-409 | ACACTGTCGTCGAATGGC |
| SEQ ID NO: 410 | AUM-A34-410 | AATGAATTCCTTTACACC |
| SEQ ID NO: 411 | AUM-A34-411 | AATACATCCATGGCTAAT |
| SEQ ID NO: 412 | AUM-A34-412 | ACAACTCCCTCCTTGGCC |
| SEQ ID NO: 413 | AUM-A34-413 | TTTCTCAGCAGCAGCCAC |
| SEQ ID NO: 414 | AUM-A34-414 | TGTCTTTCCTGCTGCTTC |
| SEQ ID NO: 415 | AUM-A34-415 | TTGTCTTTCCTGCTGCTT |
| SEQ ID NO: 416 | AUM-A34-416 | TTTGTCTTTCCTGCTGCT |
| SEQ ID NO: 417 | AUM-A34-417 | TTCTCAGCCACTGTTGCC |
| SEQ ID NO: 418 | AUM-A34-418 | CTTCTCAGCCACTGTTGC |
| SEQ ID NO: 419 | AUM-A34-419 | CTCTTTGGTCTTCTCAGC |
| SEQ ID NO: 420 | AUM-A34-420 | TCACTTGCTCTTTGGTCT |
| SEQ ID NO: 421 | AUM-A34-421 | GTCACTTGCTCTTTGGTC |
| SEQ ID NO: 422 | AUM-A34-422 | TGTCACTTGCTCTTTGGT |
| SEQ ID NO: 423 | AUM-A34-423 | ACATTTGTCACTTGCTCT |
| SEQ ID NO: 424 | AUM-A34-424 | AACATTTGTCACTTGCTC |
| SEQ ID NO: 425 | AUM-A34-425 | TGTCTTCTGGGCTACTGC |
| SEQ ID NO: 426 | AUM-A34-426 | TATCTTCCAGAATTCCTT |
| SEQ ID NO: 427 | AUM-A34-427 | ATATCTTCCAGAATTCCT |
| SEQ ID NO: 428 | AUM-A34-428 | CATATCTTCCAGAATTCC |
| SEQ ID NO: 429 | AUM-A34-429 | TTGTCAGGATCCACAGGC |
| SEQ ID NO: 430 | AUM-A34-430 | CTCATTGTCAGGATCCAC |
| SEQ ID NO: 431 | AUM-A34-431 | GTCTTGATACCCTTCCTC |
| SEQ ID NO: 432 | AUM-A34-432 | TAGTCTTGATACCCTTCC |
| SEQ ID NO: 433 | AUM-A34-433 | GTAGTCTTGATACCCTTC |
| SEQ ID NO: 434 | AUM-A34-434 | TCGTAGTCTTGATACCCT |
| SEQ ID NO: 435 | AUM-A34-435 | TTCTTAGGCTTCAGGTTC |
| SEQ ID NO: 436 | AUM-A34-436 | TTTCTTAGGCTTCAGGTT |
| SEQ ID NO: 437 | AUM-A34-437 | ATTTCTTAGGCTTCAGGT |
| SEQ ID NO: 438 | AUM-A34-438 | AGATATTTCTTAGGCTTC |
| SEQ ID NO: 439 | AUM-A34-439 | TCTCAAGAAACTGGGAGC |
| SEQ ID NO: 440 | AUM-A34-440 | TTGTACAGGATGGAACAT |
| SEQ ID NO: 441 | AUM-A34-441 | ACTTGTACAGGATGGAAC |
| SEQ ID NO: 442 | AUM-A34-442 | CACATTGGAACTGAGCAC |
| SEQ ID NO: 443 | AUM-A34-443 | TCACTGCTGATGGAAGAC |
| SEQ ID NO: 444 | AUM-A34-444 | AGATACTTCAATCACTGC |
| SEQ ID NO: 445 | AUM-A34-445 | ACAGATACTTCAATCACT |
| SEQ ID NO: 446 | AUM-A34-446 | GGTACAGATACTTCAATC |
| SEQ ID NO: 447 | AUM-A34-447 | CAGTGAAAGGGAAGCACC |
| SEQ ID NO: 448 | AUM-A34-448 | TCAGTGAAAGGGAAGCAC |
| SEQ ID NO: 449 | AUM-A34-449 | TGGTAGTCACTTAGGTGT |
| SEQ ID NO: 450 | AUM-A34-450 | ATAGTTTCATGCTCACAT |
| SEQ ID NO: 451 | AUM-A34-451 | TTATAGGTGCATAGTTTC |
| SEQ ID NO: 452 | AUM-A34-452 | ATTAAAGTGAGATGGGAT |
| SEQ ID NO: 453 | AUM-A34-453 | GTTCTTAATTCATGTTGC |
| SEQ ID NO: 454 | AUM-A34-454 | GTGTAGGGTTAATGTTCC |
| SEQ ID NO: 455 | AUM-A34-455 | AGTGTAGGGTTAATGTTC |
| SEQ ID NO: 456 | AUM-A34-456 | AGTGTTGCTTCAGGGAAT |
| SEQ ID NO: 457 | AUM-A34-457 | ACTTCTGGCAGTGTTGCT |
| SEQ ID NO: 458 | AUM-A34-458 | TAATTAATCACAGCCACT |
| SEQ ID NO: 459 | AUM-A34-459 | ATAATTAATCACAGCCAC |
| SEQ ID NO: 460 | AUM-A34-460 | CAATAATTAATCACAGCC |
| SEQ ID NO: 461 | AUM-A34-461 | ATAGTAGTTGGGGTCTTC |
| SEQ ID NO: 462 | AUM-A34-462 | AATAGTAGTTGGGGTCTT |
| SEQ ID NO: 463 | AUM-A34-463 | TACAATAGTAGTTGGGGT |
| SEQ ID NO: 464 | AUM-A34-464 | TCAGAAAGGTACAGCATT |
| SEQ ID NO: 465 | AUM-A34-465 | TTGTCAGAAAGGTACAGC |
| SEQ ID NO: 466 | AUM-A34-466 | CTACACTGCTTAGTTCCC |
| SEQ ID NO: 467 | AUM-A34-467 | TCTACACTGCTTAGTTCC |
| SEQ ID NO: 468 | AUM-A34-468 | TTCTACACTGCTTAGTTC |
| SEQ ID NO: 469 | AUM-A34-469 | TCATCTTCTACACTGCTT |
| SEQ ID NO: 470 | AUM-A34-470 | ATCATCTTCTACACTGCT |
| SEQ ID NO: 471 | AUM-A34-471 | AATCATCTTCTACACTGC |
| SEQ ID NO: 472 | AUM-A34-472 | GAATGTGCTAATATGTGC |
| SEQ ID NO: 473 | AUM-A34-473 | TCAGAGCCTTGAATGTGC |
| SEQ ID NO: 474 | AUM-A34-474 | TTTAAAGGCATTTCCTGT |
| SEQ ID NO: 475 | AUM-A34-475 | TGTTTAAAGGCATTTCCT |
| SEQ ID NO: 476 | AUM-A34-476 | ATGTTTAAAGGCATTTCC |
| SEQ ID NO: 477 | AUM-A34-477 | GATGTTTAAAGGCATTTC |
| SEQ ID NO: 478 | AUM-A34-478 | GACTCTGGTAGTTCCAAC |
| SEQ ID NO: 479 | AUM-A34-479 | AGTCTAGAGAATTGATCT |
| SEQ ID NO: 480 | AUM-A34-480 | CAGTCTAGAGAATTGATC |
| SEQ ID NO: 481 | AUM-A34-481 | ACATTTAAAGGAGGCCAT |
| SEQ ID NO: 482 | AUM-A34-482 | GGAGTTAATAGATCTTCC |
| SEQ ID NO: 483 | AUM-A34-483 | TTAGAAATGACTATGCCC |
| SEQ ID NO: 484 | AUM-A34-484 | TTTAGAAATGACTATGCC |
| SEQ ID NO: 485 | AUM-A34-485 | TACTTTCTACTAGTGACT |
| SEQ ID NO: 486 | AUM-A34-486 | ATACTTTCTACTAGTGAC |
| SEQ ID NO: 487 | AUM-A34-487 | CTCATTCCTCCTTCCTTC |
| SEQ ID NO: 488 | AUM-A34-488 | ACTCATTCCTCCTTCCTT |
| SEQ ID NO: 489 | AUM-A34-489 | TCACTCATTCCTCCTTCC |
| SEQ ID NO: 490 | AUM-A34-490 | GTCACTCATTCCTCCTTC |
| SEQ ID NO: 491 | AUM-A34-491 | GAAGTTTCTATGGTAACC |
| SEQ ID NO: 492 | AUM-A34-492 | ACTCTGTAGTAGTCTCTC |
| SEQ ID NO: 493 | AUM-A34-493 | TTCTAACCTTCCTGAAAT |
| SEQ ID NO: 494 | AUM-A34-494 | ATATCCTAACCGCCACTT |
| SEQ ID NO: 495 | AUM-A34-495 | ATATATCCTAACCGCCAC |
| SEQ ID NO: 496 | AUM-A34-496 | AAATATATCCTAACCGCC |
| SEQ ID NO: 497 | AUM-A34-497 | AAATATGCTGCTTTAGGT |
| SEQ ID NO: 498 | AUM-A34-498 | TTCTGTCCTCTATTTCTT |
| SEQ ID NO: 499 | AUM-A34-499 | AGTTCTGTCCTCTATTTC |
| SEQ ID NO: 500 | AUM-A34-500 | TAGTTCTGTCCTCTATTT |
| SEQ ID NO: 501 | AUM-A34-501 | AGTCTAGTTCTGTCCTCT |
| SEQ ID NO: 502 | AUM-A34-502 | CAGTCTAGTTCTGTCCTC |
| SEQ ID NO: 503 | AUM-A34-503 | TCAGTCTAGTTCTGTCCT |
| SEQ ID NO: 504 | AUM-A34-504 | TATCAGTCTAGTTCTGTC |
| SEQ ID NO: 505 | AUM-A34-505 | TTGTTCTAGGTCACTGCT |
| SEQ ID NO: 506 | AUM-A34-506 | ATTGTTCTAGGTCACTGC |
| SEQ ID NO: 507 | AUM-A34-507 | TGTATCCACATAAATCCT |
| SEQ ID NO: 508 | AUM-A34-508 | TTGTATCCACATAAATCC |
| SEQ ID NO: 509 | AUM-A34-509 | AGGAGAATTTGTATCCAC |
| SEQ ID NO: 510 | AUM-A34-510 | TAAAGTAATTCACTGCTC |
| SEQ ID NO: 511 | AUM-A34-511 | TTGTTTAAGTGTTTGGTC |
| SEQ ID NO: 512 | AUM-A34-512 | AATATGAGACAAGCTTCC |
| SEQ ID NO: 513 | AUM-A34-513 | TGAATATGAGACAAGCTT |
| SEQ ID NO: 514 | AUM-A34-514 | AGTGAATATGAGACAAGC |
| SEQ ID NO: 515 | AUM-A34-515 | AATGTCTCGGGAGTGAAT |
| SEQ ID NO: 516 | AUM-A34-516 | ATTGATCCTCAGGCCACT |
| SEQ ID NO: 517 | AUM-A34-517 | GATTGATCCTCAGGCCAC |
| SEQ ID NO: 518 | AUM-A34-518 | AATAACTTGGGAGAATGT |
| SEQ ID NO: 519 | AUM-A34-519 | TTAGATTGTTCTGTTCCC |
| SEQ ID NO: 520 | AUM-A34-520 | ATTAGATTGTTCTGTTCC |
| SEQ ID NO: 521 | AUM-A34-521 | CATTAGATTGTTCTGTTC |
| SEQ ID NO: 522 | AUM-A34-522 | CTACATACTGGATAAGCC |
| SEQ ID NO: 523 | AUM-A34-523 | GCTACATACTGGATAAGC |
| SEQ ID NO: 524 | AUM-A34-524 | AATAGCTACATACTGGAT |
| SEQ ID NO: 525 | AUM-PD-F- | FU*FA*FU*FA*FU*FU*C* |
| | 001 | C*C*A*G*C*T*C*C*FC* |
| | | FU*FC*FC*FA*FC |
| SEQ ID NO: 526 | AUM-PD-F- | FA*FC*FA*FA*FU*FU*T* |
| | 001 | A*A*G*T*G*T*G*A*FA* |
| | | FG*FC*FC*FA*FC |
| SEQ ID NO: 527 | AUM-PD-F- | FA*FA*FC*FC*FU*FA*C* |
| | 001 | A*T*A*G*A*G*G*A*FC* |
| | | FU*FC*FC*FC*FU |
| SEQ ID NO: 528 | AUM-PD-F- | FU*FA*FU*FA*FU*FU*C* |
| | 001 | C*C*A*G*C*T*C*C*FC* |
| | | FU*FC*FC*FA*FC |
| SEQ ID NO: 529 | AUM-PD-F- | FC*FU*FU*FU*FC*FA*T* |
| | 001 | G*A*A*C*A*C*A*T*FC* |
| | | FC*FA*FU*FG*FG |
| SEQ ID NO: 530 | AUM-PD-F- | FC*FC*FU*FU*FU*FC*A* |
| | 001 | T*G*A*A*C*A*C*A*T*C* |
| | | FC*FA*FU*FG*FG*FC |
| SEQ ID NO: 531 | AUM-PD-001 | TATATTCCCAGCTCCCTCCAC |
| SEQ ID NO: 532 | AUM-PD-002 | ACAATTTAAGTGTGAAGCCAC |
| SEQ ID NO: 533 | AUM-PD-003 | AACCTACATAGAGGACTCCCT |
| SEQ ID NO: 534 | AUM-PD-004 | TATATTCCCAGCTCCCTCCAC |
| SEQ ID NO: 535 | AUM-PD-005 | CTTTCATGAACACATCCATGG |
| SEQ ID NO: 536 | AUM-PD-006 | CCTTTCATGAACACATCCATG |
| | | GC |

In one aspect, the α-synuclein targeting FANA-ASO oligonucleotide has the nucleic acid sequence of SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 2, 30, 31, 32, 33, 33, 34, 35, 36, 37, 38, 39, 40, 41, 42, 43, 44, 45, 46, 47, 4, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 492, 493, 493, 495, 496, 497, 498, 499, 500, 501, 502, 503, 504 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536 or a combination thereof.

### FANA-ASO Compositions

In some aspects, the oligonucleotide sequence is a complement to the sequence of the RNA, and the oligonucleotide sequence has at least 80%, 85%, 90%, 95%, 98%, 99%, or more sequence identity to the complementary sequence of the target RNA.

As used herein, the terms “complementary” or “complementarity” are used in reference to polynucleotides (i.e., a sequence of nucleotides) related by the base-pairing rules. For example, the sequence 5′-A-G-T-3′, is complementary to the sequence ″′-T-C-A-5′. Complementarity may be “partial”, in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. As such, a “complement” sequence, as used herein refers to an oligonucleotide sequence have some complementarity to a target RNA or DNA sequence. The complementarity between the target RNA or DNA and the oligonucleotide can be at least 80%, 81%, 82%, 83%, 84%, 85%, 86%, 87%, 88%, 89%, 90%, 91%, 92%, 93%, 94%, 95%, 96%, 97%, 98%, 99%, or 100%. In certain aspects the target RNA or DNA is RNA or DNA encodes α-synuclein.

For the purpose of the invention, the “complement of a nucleotide sequence X” is the nucleotide sequence which would be capable of forming a double-stranded DNA or RNA molecule with the represented nucleotide sequence, and which can be derived from the represented nucleotide sequence by replacing the nucleotides by their complementary nucleotide according to Chargaff's rules (A<>T; G<>C; A<>U) and reading in the 5′ to 3′ direction, i.e., in opposite direction of the represented nucleotide sequence. In the context of the present disclosure, this term also includes synthetic analogs of DNA/RNA (e.g., 2′F-ANA oligos).

The term “homology” or “identity” refers to a degree of complementarity. There may be partial homology or complete sequence identity between the oligonucleotide sequence and the complement sequence of the target RNA or DNA. A partially identical sequence is an oligonucleotide that at least partially hybrids to the target RNA or DNA, leading to the formation of partial heteroduplex, and to partial or total degradation of the target RNA or DNA. A completely identical sequence is an oligonucleotide that completely hybrids to the target RNA or DNA, leading to the formation of complete heteroduplex, and to partial or total degradation of the target RNA or DNA.

In various aspects, the target RNA or DNA is selected from the group consisting of messenger RNA (mRNA), microRNA (miRNA), small interfering (siRNA), antisense RNA (aRNA), short hairpin RNA (shRNA), transfer RNA (tRNA), ribosomal RNA (rRNA), small nuclear RNA (snRNA), double-stranded RNA (dsRNA), locked nucleic acid (LNA), Transfer-messenger RNA (tmRNA), viral RNA, viral DNA, polynucleic acids circular ssDNA, and circular DNA.

As used herein, the term “nucleic acid” refers to polynucleotides such as deoxyribonucleic acid (DNA) or ribonucleic acid (RNA), with RNA being prepared or obtained by the transcription a DNA template. According to the invention, a nucleic acid may be present as a single-stranded or double-stranded and linear or covalently circularly closed molecule.

In other aspects, the oligonucleotide sequence has at least 80%, 85%, 90%, 95%, 98%, 99%, or more sequence identity to the complementary RNA or DNA sequence such as an RNA or DNA sequence encoding α-synuclein.

In an additional embodiment, the present invention provides a pharmaceutical composition with an α-synuclein targeting FANA-ASO oligonucleotide and a pharmaceutically acceptable carrier. In one aspect, the α-synuclein targeting FANA-ASO oligonucleotide has a nucleic acid sequence selected from the group consisting of SEQ ID NOs: 1-536 or a combination thereof. In an additional aspect, the α-synuclein targeting FANA-ASO oligonucleotide has at least one 2′FANA modified nucleotide. In a further aspect, the at least one 2′FANA modified nucleotide is positioned within the oligonucleotide according to any of Formula 1-16. In certain aspects, the pharmaceutically acceptable carrier is phosphate buffer; citrate buffer; ascorbic acid; methionine; octadecyldimethylbenzyl ammonium chloride; hexamethonium chloride; benzalkonium chloride; benzethonium chloride; phenol alcohol; butyl alcohol; benzyl alcohol; methyl paraben; propyl paraben; catechol; resorcinol; cyclohexanol; 3-pentanol; m-cresol; low molecular weight (less than about 10 residues) polypeptides; serum albumin; gelatin; immunoglobulins; polyvinylpyrrolidone glycine; glutamine; asparagine; histidine; arginine; lysine; monosaccharides; disaccharides; glucose; mannose; dextrins; EDTA; sucrose; mannitol; trehalose; sorbitol; sodium; saline; metal surfactants; non-ionic surfactants; polyethylene glycol (PEG); magnesium stearate; water; alcohol; saline solution; glycol; mineral oil or dimethyl sulfoxide (DMSO).

As used herein, “pharmaceutical composition” refers to a formulation comprising an active ingredient, and optionally a pharmaceutically acceptable carrier, diluent or excipient. The term “active ingredient” can interchangeably refer to an “effective ingredient”, and is meant to refer to any agent that is capable of inducing a sought-after effect upon administration. Examples of active ingredient include, but are not limited to, chemical compound, drug, therapeutic agent, small molecule, etc.

By “pharmaceutically acceptable” it is meant the carrier, diluent or excipient must be compatible with the other ingredients of the formulation and not deleterious to the recipient thereof, nor to the activity of the active ingredient of the formulation. Pharmaceutically acceptable carriers, excipients or stabilizers are well known in the art, for example Remington's Pharmaceutical Sciences, 16th edition, Osol, A. Ed. (1980). Pharmaceutically acceptable carriers, excipients, or stabilizers are nontoxic to recipients at the dosages and concentrations employed, and may include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (for example, Zn-protein complexes); and/or non-ionic surfactants such as TWEEN™, PLURONICS™ or polyethylene glycol (PEG). Examples of carrier include, but are not limited to, liposome, nanoparticles, ointment, micelles, microsphere, microparticle, cream, emulsion, and gel. Examples of excipient include, but are not limited to, anti-adherents such as magnesium stearate, binders such as saccharides and their derivatives (sucrose, lactose, starches, cellulose, sugar alcohols and the like) protein like gelatin and synthetic polymers, lubricants such as talc and silica, and preservatives such as antioxidants, vitamin A, vitamin E, vitamin C, retinyl palmitate, selenium, cysteine, methionine, citric acid, sodium sulfate and parabens. Examples of diluent include, but are not limited to, water, alcohol, saline solution, glycol, mineral oil and dimethyl sulfoxide (DMSO).

In one aspect, the α-synuclein targeting FANA-ASO oligonucleotide has the nucleic acid sequence of SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 2, 30, 31, 32, 33, 33, 34, 35, 36, 37, 38, 39, 40, 41, 42, 43, 44, 45, 46, 47, 4, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 492, 493, 493, 495, 496, 497, 498, 499, 500, 501, 502, 503, 504 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536 or a combination thereof.

In a further embodiment, the present invention provides a method of decreasing α-synuclein expression by administering an α-synuclein targeting FANA-ASO oligonucleotide to a subject in need thereof, thereby reducing α-synuclein expression. In one aspect, the α-synuclein expression is decreased in neurons, oligodendrocytes and/or astrocytes. In an additional aspect, the α-synuclein targeting FANA-ASO oligonucleotide has at least one 2′FANA modified nucleotide. In certain aspects, the at least one 2′FANA modified nucleotide is positioned within the oligonucleotide according to any of Formula 1-16. In various aspects, the α-synuclein targeting FANA-ASO oligonucleotide has a nucleic acid sequence of SEQ ID NOs: 1-536 or a combination thereof. In a further aspect, the α-synuclein targeting FANA-ASO oligonucleotide has the nucleic acid sequence of SEQ ID NOs:525 or 527. In certain aspects, the α-synuclein targeting FANA-ASO oligonucleotide is administered by intracutaneous, subcutaneous, intravenous, intraperitoneal, intraarterial, intrathecal, intracapsular, intraorbital, intracardiac, intradermal, transdermal, transtracheal, subcuticular, intraarticular, intracerebroventricular, subcapsular, subarachnoid, intraspinal, intrasternal, oral, sublingual buccal, rectal, vaginal, ocular, inhalation, or nebulization.

In one aspect, the α-synuclein targeting FANA-ASO oligonucleotide has the nucleic acid sequence of SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 2, 30, 31, 32, 33, 33, 34, 35, 36, 37, 38, 39, 40, 41, 42, 43, 44, 45, 46, 47, 4, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 492, 493, 493, 495, 496, 497, 498, 499, 500, 501, 502, 503, 504 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536 or a combination thereof.

Alpha synuclein expression levels can be determine by any method known in the art including western blot assay, ELISA assay, flow cytometry or other fluorescence-based assays.

In another embodiment, the present invention provides a method of reducing Lewy body and/or Lewy neurite pathology by administering an α-synuclein targeting FANA-ASO oligonucleotide to a subject in need thereof, thereby decreasing Lewy body and/or Lewy neurite pathology. In one aspect, the reduction of the Lewy body and/or Lewy neurite pathology is in neurons, oligodendrocytes and/or astrocytes. In an additional aspect, the α-synuclein targeting FANA-ASO oligonucleotide has at least one 2′FANA modified nucleotide. In certain aspects, the at least 2′FANA modified nucleotide is positioned within the oligonucleotide according to any of Formula 1-16. In various aspects, the α-synuclein targeting FANA-ASO oligonucleotide has a nucleic acid sequence of SEQ ID NOs:1-536 or a combination thereof. In a further aspect, the α-synuclein targeting FANA-ASO oligonucleotide has the nucleic acid sequence of SEQ ID NO: 525 or 527.

In one aspect, the α-synuclein targeting FANA-ASO oligonucleotide has the nucleic acid sequence of SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 2, 30, 31, 32, 33, 33, 34, 35, 36, 37, 38, 39, 40, 41, 42, 43, 44, 45, 46, 47, 4, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 492, 493, 493, 495, 496, 497, 498, 499, 500, 501, 502, 503, 504 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536 or a combination thereof.

As used herein the terms “α-Synucleinopathies” and “α-synuclein pathologies” are used interchangeably and refer to neurodegenerative diseases characterized by the abnormal accumulation of aggregates of alpha-synuclein protein in neurons, nerve fibers or glial cells. There are three main types of α-synuclein pathologies: Parkinson's disease (PD), dementia with Lewy bodies (DLB), Alzheimer's Disease and multiple system atrophy (MSA).

Parkinson's Disease is characterized by α-synuclein pathology—Lewy bodies (LBs) and Lewy neurites (LNs) are the neuropathological hallmarks of PD, PDD and DLB, a related disorder distinguished by the onset of dementia prior to classical Parkinsonism. These intraneuronal inclusions are comprised of aggregated α-synuclein, a heat-stable 140 amino acid long protein expressed ubiquitously in a variety of tissues including neurons and erythrocytes. Importantly, point mutations or amplification of the SNCA locus cause autosomal dominant forms of familial PD.

The role that LBs/LNs play in synucleinopathies remains unclear. However, extensive post-mortem studies on the neuroanatomical distribution of LBs/LNs in PD/PDD/DLB have revealed several important concepts. Firstly, LBs/LNs affect multiple CNS regions that vary with different synuclein pathologies and even within one disorder such as PD, although significant overlaps exist. Secondly, motor and non-motor symptoms strongly correlate with the extent of α-synuclein pathology and the function of these affected areas. Thirdly, α-synuclein pathology progressively accumulates, affecting new CNS regions over time, while pathology in previously affected areas increases in severity. For example, in PD LBs/LNs first develop in lower brainstem nuclei, olfactory nuclei, and peripheral neurons of the skin and gut coinciding with prodromal symptoms that are mainly gastrointestinal, sensory and sleep related. LBs/LNs in the midtemporal cortex are associated with hallucinations, while the appearance of midbrain LBs coincides with the start of classical motor symptoms, followed by neocortical involvement which typically occurs last. Although some patients deviate from this pattern, the majority of patients appear to exhibit this stereotypic progression of α-synuclein pathology.

Alpha-synuclein pathology propagates in PD. The progressive and sequential spread of LBs/LNs from affected to unaffected CNS regions over time is consistent with the transmission of a pathogenic agent or process from diseased to healthy neurons. In fact, LBs/LNs are frequently detected in gastrointestinal, cardiac, as well as olfactory neurons during early stages of PD, suggesting that spread might occur over long distances and that the initiating pathogenic event may be environmental in origin. Brainstem nuclei, such as the dorsal motor nucleus of the vagus (DMV), might then serve as intermediary sites for the progression of this pathogenic process and LBs/LNs to higher regions like mesencephalon and neocortex. Indeed, vagotomy appears to be protective against PD in humans. One of the first clues that the transmissible agent in PD might be α-synuclein itself comes from post-mortem studies showing the time-dependent formation of LBs in mesencephalic neurons grafted into PD patients. More recently it was demonstrated that synthetic α-synuclein PFFs seeded the formation of insoluble PD-like LBs/LNs in α-synuclein-expressing cells, including cultured neurons. Congruent with LBs/LNs being detrimental, this PD-like α-synuclein pathology induces synaptic dysfunction and ultimately cell death in cultured hippocampal neuron. It has been shown that intracerebral injection of mouse (Mse) α-synuclein PFFs into wild type mice from a variety of genetic backgrounds induces formation of abundant LBs/LNs in multiple connected regions, including SNpc, which progressively degenerates as LBs/LNs accumulate, resulting in loss of striatal DA and impaired motor function. Biochemical analysis shows that α-synuclein PFFs trigger the pathological conversion of host-expressed α-synuclein, whereas PFFs are non-toxic and do not induce pathology in the absence of α-synuclein expression in Snca^{-/-} mice.

As opposed to cell-surface or secreted proteins, propagation along axons is a logical candidate for intracellular proteins such as α-synuclein and tau. Examination of brains from mice following α-synuclein PFF injections showed that LB/LN formation occurs initially at the site of injection, but quickly disseminates to additional afferent and efferent neurons connected to the injection site. In transgenic mice overexpressing A53T human α-synuclein (M83 line), PFFs injected into the striatum and cortex develop considerable pathology in thalamus, brain stem, but also in frontal cortical regions, where pathology is scant or absent in non-injected symptomatic M83 animals. These animals also showed LBs/LNs in multiple nuclei located at considerable distances from and contralateral to the injection sites, including those lacking direct input/output projections (e.g., spinal cord and deep cerebellar nuclei), consistent with cell-to-cell spread of pathological α-synuclein. Abundant α-synuclein deposits also developed along intermediary white matter tracts, suggesting that pathology propagated along axonal pathways, and possibly across synapses. The observation that pathology preferentially formed in neurons projecting either to or from the injection site also applied to wild type mice. For example, dorsal striatal PFF injections produced prominent pathology in SNpc (unilateral), cortical layers 4/5 (bilateral), and amygdala (bilateral), in agreement with established nigrostriatal, corticostriatal, and amygdalostriatal pathways. Inclusions were also detected in some neurons lacking direct connections with the injection site (e.g. olfactory mitral cells), suggestive of α-synuclein pathology spread across multiple synapses. Moreover, PFF injections into hippocampus resulted in LB/LN formation in multiple cortical regions and amygdala, while sparing most subcortical, midbrain and brainstem structures. Thus, α-synuclein PFFs exhibit all the key features of transmissible self-propagating agents that induce toxicity through LB/LN formation. Indeed, misfolded α-synuclein displays elements characteristic of prions with the notable exception of infectivity.

The terms “therapeutically effective amount”, “effective dose,” “therapeutically effective dose”, “effective amount,” or the like refer to that amount of the subject compound that will elicit the biological or medical response of a tissue, system, animal or human that is being sought by the researcher, veterinarian, medical doctor or other clinician. Generally, the response is either amelioration of symptoms in a patient or a desired biological outcome.

The terms “administration of” and or “administering” should be understood to mean providing a pharmaceutical composition in a therapeutically effective amount to the subject in need of treatment. Administration routes can be enteral, topical or parenteral. As such, administration routes include but are not limited to intracutaneous, subcutaneous, intravenous, intraperitoneal, intraarterial, intrathecal, intracapsular, intraorbital, intracardiac, intradermal, transdermal, transtracheal, subcuticular, intraarticular, intracerebroventricular, subcapsular, subarachnoid, intraspinal, intrasternal, oral, sublingual buccal, rectal, vaginal, ocular, inhalation, or nebulization. The phrases “parenteral administration” and “administered parenterally” as used herein means modes of administration other than enteral and topical administration.

In one embodiment, the present invention provides a method of preventing and/or treating Parkinson's Disease or symptoms thereof, by administering an α-synuclein targeting FANA-ASO oligonucleotide to a subject in need thereof, thereby preventing and/or treating Parkinson's Disease. In one aspect, the administration of the α-synuclein targeting FANA-ASO oligonucleotide decreases expression of α-synuclein in cells. In certain aspects, the cells are neurons; oligodendrocytes and/or astrocytes. In various aspects, the α-synuclein targeting FANA-ASO oligonucleotide is administered by intracutaneous, subcutaneous, intravenous, intraperitoneal, intraarterial, intrathecal, intracapsular, intraorbital, intracardiac, intradermal, transdermal, transtracheal, subcuticular, intraarticular, intracerebroventricular, subcapsular, subarachnoid, intraspinal, intrasternal, oral, sublingual buccal, rectal, vaginal, ocular, infusion, inhalation, or nebulization. In one aspect, the subject is human. In an additional aspect, the α-synuclein targeting FANA-ASO oligonucleotide has at least one 2′FANA modified nucleotide. In a further aspect, the at least one 2′FANA modified nucleotide is positioned within the oligonucleotide according to any of Formula 1-16. In certain aspects, the α-synuclein targeting FANA-ASO oligonucleotide has a nucleic acid sequence of SEQ ID NOs:1-536. In a further aspect, the α-synuclein targeting FANA-ASO oligonucleotide has the nucleic acid sequence of SEQ ID NO: 525 or 527. In another aspect, Lewy body and/or Lewy neurite pathology is reduced. In an additional aspect, a therapeutic agent is administered. In a further aspect, the therapeutic agent is administered prior to, simultaneously with, or following administration of the α-synuclein targeting FANA-ASO oligonucleotide. In a specific aspect, the therapeutic agent is Levodopa.

In one aspect, the α-synuclein targeting FANA-ASO oligonucleotide has the nucleic acid sequence of SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 2, 30, 31, 32, 33, 33, 34, 35, 36, 37, 38, 39, 40, 41, 42, 43, 44, 45, 46, 47, 4, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 492, 493, 493, 495, 496, 497, 498, 499, 500, 501, 502, 503, 504 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536 or a combination thereof.

The term “effective amount” of a composition provided herein refers to the amount of the composition capable of performing the specified function for which an effective amount is expressed. The exact amount required can vary from composition to composition and from function to function, depending on recognized variables such as the compositions and processes involved. An effective amount can be delivered in one or more applications. Thus, it is not possible to specify an exact amount, however, an appropriate “effective amount” can be determined by the skilled artisan via routine experimentation.

As used herein, “preventing” a disease refers to inhibiting the full development of a disease.

The term “treatment” is used interchangeably herein with the term “therapeutic method” and refers to both 1) therapeutic treatments or measures that cure, slow down, lessen symptoms of, and/or halt progression of a diagnosed pathologic conditions or disorder, and 2) and prophylactic/preventative measures. Those in need of treatment may include individuals already having a particular medical disorder as well as those who may ultimately acquire the disorder (i.e., those needing preventive measures).

In some aspects administration can be in combination with one or more additional therapeutic agents. The phrases “combination therapy”, “combined with” and the like refer to the use of more than one medication or treatment simultaneously to increase the response. The composition of the present invention might for example be used in combination with other drugs or treatment in use to treat Parkinson's Disease.

The following examples are provided to further illustrate the embodiments of the present invention but are not intended to limit the scope of the invention.

### EXAMPLES

### Example 1

### FANA-ASO Mediated Knockdown of α-Synuclein-GFP in Mouse Neurons

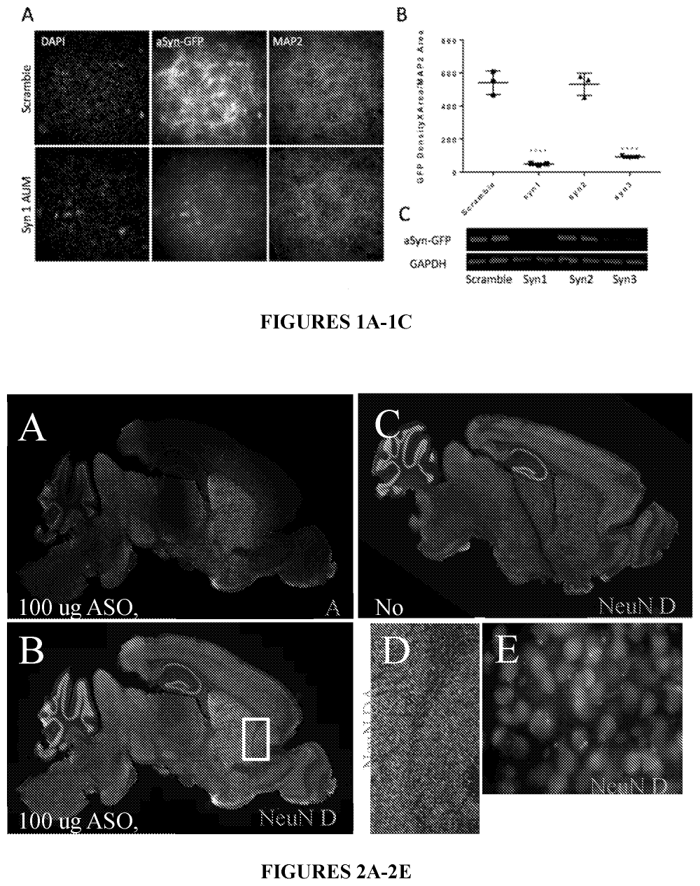

FANA-ASOs were screened against SNCA gene to identify the most potent FANA-ASOs. Primary cortical neuron cultures were prepared from postnatal day 1 α-synuclein-GFP knock-in (Snca^{GFP/GFP}) mice and plated at 60,000 cells cm⁻² on poly-D-lysine coated 96-well plates. At 7 days in vitro (DIV), cultures were treated with FANA-ASO targeting α-synuclein (Syn1/AUM) or a scrambled sequence at a final concentration of 1 μM. Neurons were imaged 14 days after treatment with FANA-ASOs and α-synuclein-GFP levels visualized using fluorescence microscopy in the GFP channel at 20× magnification (FIG. 1A). Fluorescence levels of α-synuclein-GFP in neurons treated with different FANA-ASO sequences were quantified. Data represent values from individual wells (+/−SD; n=3 per condition) (FIG. 1B). protein levels were evaluated to determine if there was a correlation with the analysis and observe similar patterns. Over 90% knockdown of SNCA was observed by two FANA-ASOs which were identified as lead compounds (FIG. 1C).

### Example 2

### Biodistribution of FANA-ASOS in Vivo

One of the most important aspect for any therapeutic modality is efficient in vivo delivery. FANA-ASOs are able to enter several types of cells without delivery formulations or conjugates. Further, FANA-ASOs can be used in vivo via multiple modes of administration. In a preliminary study, FANA-ASOs were evaluated for the ability to self-deliver to neurons and non-neuronal cells in the cerebral cortex of the animal. Broad and efficient distribution of FANA-ASOs was observed in mouse brain by intracerebroventral injection (FIG. 2). FANA-ASO containing a scrambled sequence was labeled with the fluorescent dye Cy5 and injected into adult C57B16/C3H mice. Injection was made via a single intracerebroventricular (i.c.v.) injection into the right ventricle (100 μg total FANA-ASO in 10 μL PBS) using a 32-gauge Hamilton syringe connected to a Neurostar digital injection unit. Mice (n=2) were sacrificed 48 h later and FANA-ASO detected by fluorescence microcopy using Cy5 filter set to visualize distribution in the brain (FIGS. 2A-B). Immunostaining against NeuN was performed to reveal neurons and DAPI was used to label cell nuclei. FANA-ASO was detected in the cerebral cortex and striatum of the injected mice (FIG. 2B highlighted panel) High powered micrographs shows FANA-ASO within the cell bodies of NeuN-labeled neurons and non-neuronal cells in the cerebral cortex.

### Example 3

### FANA-ASO Mediated Knockdown of α-Synuclein Reduces Fibril-Induced Lewy-Like Pathologyin Neurons

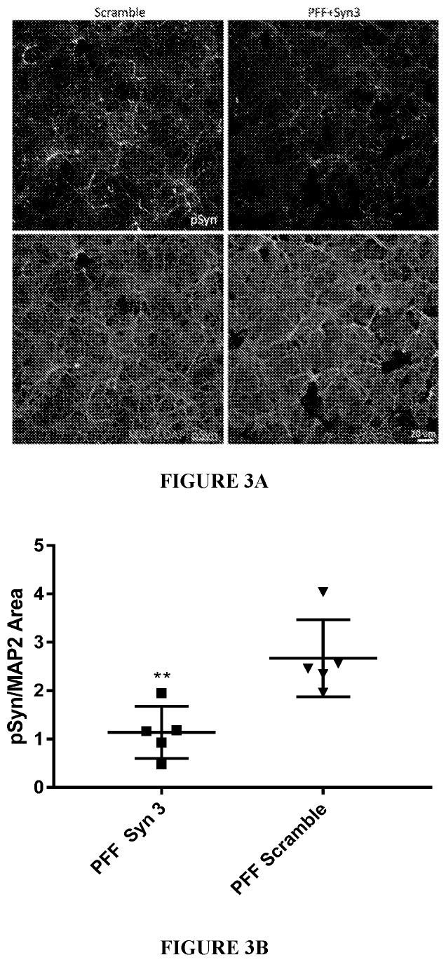

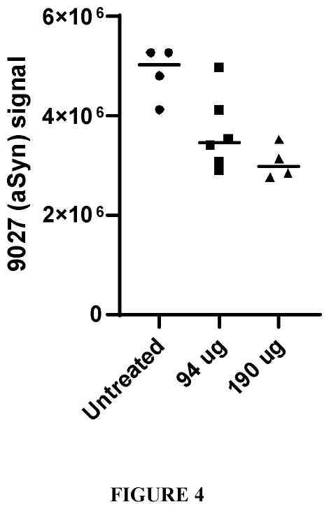

Experiments were performed to determine if the knockdown of SNCA reduces fibril-induced Lewy-like pathology in neurons. Primary hippocampal neurons prepared from embryonic day 16-18 wild type (CD-1) mice and plated onto poly-D-lysine coated 96-well plates. FANA-ASO targeting α-synuclein (Syn3/AUM; 1 μM final concentration) or a scrambled sequence was added to cultures at DIV 8. Recombinant α-synuclein pre-formed fibrils (PFFs; 70 nM final concentration) to induce Lewy-like pathology were added 4 h later. Neurons were fixed with 4% PFA at 12 d after treatment with PFF/FANA-ASOs. Pathological α-synuclein inclusions were visualized using fluorescence immunocytochemistry for α-synuclein phosphorylated at Ser129 (pSyn; mAb clone 81A). Cultures were co-stained with an antibody against microtubule associated protein-2 (MAP2) to reveal neuronal cell bodies and processes (FIG. 3A). Further, phosphor-Ser129 α-synuclein levels were quantified in neurons co-treated with PFFs and either Syn3 or scrambled FANA-ASO from 20× images of the cultures (FIG. 3B). Data represent means from mean phosphor-Ser129 α-synuclein levels normalized to MAP2+/−SD (n=5 per condition). ** p<0.01 two-tailed t-test.

### Example 4

### Optimization of Next Generation Self-Deliverable FANA Sequences

The above described studies have shown that FANA-ASOs can effectively inhibit SNCA gene and selectively inhibit production of α-synuclein. This reduced fibril-induced Lewy-like pathology in neurons. Two FANA-ASO sequences have been identified that decreases SNCA gene expression by over 90%, other lead compounds will be identified and optimized. Each FANA-ASO comprises two factors: the sequence and the design. The sequence is the actual order of nucleotide base pairs that will make up the oligo. A proprietary algorithm determines which DNA sequences are most likely to be stable and efficacious while minimizing immune response. The design not only encompasses if it is RNase H active or inactive, but whether each nucleotide on the oligo is a DNA nucleotide or a modified FANA nucleotide. It is worth testing a wide variety of possible sequences, as even a single base pair change can result in wildly different results. The chemistry of the current generation of FANA technology, specifically the stereo-electronic effects linked to the FANA's fluorine, provide these oligonucleotides with highly sequence specific and enhanced hybridization to their RNA target. Studies have demonstrated that FANA-ASOs can be designed to have target specificity to a single nucleotide Watson-Crick base pair resolution. This specificity was further demonstrated in a study of chronic obstructive pulmonary disease (COPD); FANA-ASOs designed with one base pair mismatch to the target sequences resulted in complete loss of function. In vitro assays will be performed to identify prospective compounds. New FANA-ASO sequences will be designed that will target different regions of the SNCA mRNA. Additionally, the efficacy of FANA-ASOs in human cell lines that naturally express α-synuclein and iPSC-derived neurons will be evaluated. The chemistry of the current generation of FANA technology, specifically the stereo-electronic effects linked to the FANA's fluorine, provide these oligonucleotides with highly sequence specific and enhanced hybridization to their RNA target.

The preliminary studies involved ˜10 sequences, and it is very possible a more optimal sequence exists. Approximately 20 new FANA-ASOs will be developed against SNCA and compare their utility on knockdown of SNCA, using AUM-PD-001 and AUM-PD-003 as a key control. SNCA mRNA and protein levels will be quantified by qPCR and Western blot respectively, along with their ability to reduced fibril-induced Lewy-like pathology in neurons. Additionally, studies will be performed to increase FANA stability and function by testing the effects of differing FANA gapmer and altimer designs, including the AUM-PD-001 and AUM-PD-003 (SEQ ID NOs:7 and 9) lead compounds and 1-2 backup ASO selected from the new studies. The length and order of FANA modified bases can be easily changed, which could have the ability to drastically alter silencing profiles.

FANA ASOs will be screened against α-synuclein-GFP neurons at 3, 7 and 10 d after a single treatment as described above. Each FANA ASOs will be tested at 7 concentrations (5, 25, 100, 500, and 5,000 nM). Scrambled FANA ASO will be used as negative controls. Active FANA ASOs will be defined as those show knockdown efficiencies or IC50 values equal to or exceeding AUM-PD-001 and -003. All cell-based experiments will be run in 96-well plate format with ≥3 independent trials where each condition is tested in ≥3 wells per run. Knockdown will then be confirmed using qPCR and western blot to measure SNCA mRNA and protein levels, respectively. Confirmed FANA ASOs will also be tested for their ability to reduce recombinant mouse PFF-induced pathology in wildtype neurons as described above. Another goal is to increase FANA stability and function by testing the effects of differing FANA gapmer and altimer designs, including AUM-PD-001 and AUM-PD-003 lead compounds and 1-2 backup ASO selected from the new studies. The length and order of FANA modified bases can be easily changed, which could have the ability to drastically alter silencing profiles.

Although all FANA ASOs tested are expected to target human SNCA in silico, newly identified candidates will be evaluated to ensure that the sequences effectively knockdown α-synuclein in human SK-MEL30 melanoma cells and iPSC-differentiated cortical layer 5 glutamatergic neurons (BrainXell, Madison WI). Both cell types have been shown to naturally express α-synuclein. The majority of FANA ASOs identified will knockdown α-synuclein in both mouse and human cells. Given that self-delivery into neurons is a criterion for selection, qPCR and western blotting will be used to confirm downregulation in human cells. It has been shown that human α-synuclein aggregates ˜10-fold slower than murine α-synuclein, hence although PFF-seeding has been shown in iPSC neurons, will not test FANA ASOs in this context here.

### Example 5

### Evaluation of Lead FANA-ASO Compounds in an in Vivo Model of PD

The distribution of Lewy pathology in PD patients correlates closely with the nature and severity of their symptoms. LBs/LNs evolve in a non-uniform and stereotypic pattern consistent with the sequential spread of pathological α-synuclein from affected to unaffected CNS regions over time. It has been shown that stereotaxic injection of mouse α-synuclein PFFs into the dorsal striatum of non-transgenic mice induces formation of abundant Lewy pathology in inter-connected regions, including the substantia nigra, which progressively degenerates, resulting in loss of striatal dopamine and impaired motor function. Biochemical analysis shows that α-synuclein PFFs trigger the pathological conversion of host-expressed α-synuclein, whereas PFFs are non-toxic and do not induce pathology in the absence of α-synuclein expression in Snca^{1/1} mice 7. Importantly, these models have also been replicated in rats, marmosets, and macaques. Studies showing that altered α-synuclein species are elevated in cerebrospinal fluid of PD patients and that homogenates isolated from brains of PD and DLB patients seed Lewy pathology in rodents and non-human primates indicate that seeding-competent α-synuclein species are present in human PD. Validation of whether candidate FANA ASOs knockdown α-synuclein levels in vivo and provide protection against α-synuclein-mediated neurodegeneration by reducing the formation of Lewy pathology.

To determine dosing for α-synuclein knockdown, 2-3 months old wt mice (C57BL6/C3H F1; Jackson Laboratories) will be treated with in one hemisphere with a single dose of the lead or scrambled FANA ASO (100, 300, or 700 μg, i.c.v.). Cohorts (n=3 of each sex) will be sacrificed 1 month after treatment. Each hemisphere is then dissected and assayed for Snca mRNA and α-synuclein protein. To assess neuroprotection by FANA ASOs, single unilateral injections of recombinant mouse α-synuclein PFFs (5 μg α-synuclein in 2 μL PBS) will be targeted to the dorsal striatum of young wt mice. Previously validated conditions will be used to generate PFFs that have high α-synuclein seeding capacity and do not cross-seed other proteins such as tau. Stereotactic methods have been established. Animals injected with PBS will be used as negative pathology controls. Two weeks prior to PFF inoculation, cohorts will receive a single dose of either lead or scrambled FANA ASO into the hemisphere that will be seeded with pathology. Cohorts will undergo motor behavior analysis (rotarod and wire-hang tests) at either 3 or 6 months post-injection and then sacrificed for histological assessment of the brain. These timepoints represent peak α-synuclein pathology and maximal nigral neuron loss as previously determined. Twelve animals will be used per cohort based on a need to detect a >20% difference in pathology or neuron number (at 0.05 level and 0.8 power) and assuming a CV of ˜15% observed in previous work. PFA (4%)-fixed brains are sectioned at 40 μm using a compresstome. A 1:6 series of sections will be immunostained with a panel of α-synuclein antibodies, including anti-phospho α-synuclein (phospho-Ser129 α-synuclein) and Syn506 that were demonstrated previously to preferentially stain Lewy pathology over normal synaptic α-synuclein in human brains. Staining with a pan-α-synuclein antibody (SNL4) will be used to confirm knockdown consistent with initial dosing studies. Adjacent section series will be stained for tyrosine hydroxylase (TH) to label dopamine neurons with a Nissl counterstain for stereological quantification to determine nigral dopamine neuron loss. Images will be digitized (Lamina scanner, Perkin-Elmer) and will be used to extract histological data, such as distribution/number of α-synuclein+inclusions.

### Example 6

### Genetic Toxicology, Pharmacokinetic and ADME Studies of Lead Compound

In some studies, significant effects of FANA administration on liver transaminases, renal function (BUN, Cr), hematologic parameters, colitis, hyperglycemia or histologic features consistent with toxicity or induction of autoimmunity have not been seen. However, the lead compounds need to be assessed for drug metabolism, pharmacology, and toxicity parameters. Some small-scale PK/PD and ADME studies will be performed to define the therapeutic space and inform further optimization. Metabolic stability and metabolite identification will also be performed along with plasma protein binding assays. A minimum number of animals will be used, and standard in vivo PK/PD tests run to begin to characterize the lead compound.

PK data obtained from 6-8 wk old rats are used as the basis for setting dose and frequency of dosing in safety pharmacology and toxicology studies, to characterize differences in ADME in higher species when compared to rodents, and in prediction of pharmacokinetic parameters such as clearance and volume of distribution in humans using allometric scaling. Blood samples will be collected at pre-dose and at 0.083, 0.25, 0.5, 1, 2, 4, 8, and 24 hours post-dose, plus urine at 24 hours, and FANA levels will be determined by LCMSMS, along with data on plasma protein binding. Cross-species metabolism in hepatocytes will be assessed in vitro In vitro genotoxicity tests including (but not limited to) bacterial reverse mutation (Ames) test, In Vitro micronucleus test, and rodent bone marrow micronucleus test will be performed. Lack of genotoxic effects in this model will be considered to decrease the risk of molecule failure at later development stages.

### Example 7

### Reduction of SNCA by Self-Delivering FANA-ASOS in Human Cell Lines That Naturally Express α-Synuclein and in IPSC-Derived Neurons

FANA-ASOs offer unique advantages, including self-delivery, over other RNA silencing technologies. Additionally, FANA-ASOs do not cause cytotoxicity or immune response. Unlike RNAi or CRISPR approaches, FANA-ASOs do not require delivery agents to be taken up by cells (including difficult to target immune cells) both in vitro and in animal studies. Further FANA-ASOs do not cause any cytotoxicity and have no apparent immune response. To this end, the capability of FANA-ASOs to achieve sequence specific inhibition of SNCA gene in human cell lines that naturally express α-synuclein and in iPSC-derived neurons will be evaluated.

### Example 8

### Knockdown of SNCA Causes Inhibition of α-Synuclein Production and Lewy-Like Pathology in Models of PD

FANA-ASOs can be used in vivo to silence a wide variety of RNA targets in a highly sequence specific manner. It will be shown that the knock down of SNCA with a third generation ASO chemistry which will have much superior efficacy than existing ASO chemistries. Knockdown of SNCA will potentially lead to the prevention of the disease by inhibition of α-synuclein production and reduction of α-synuclein pathology. Inhibition of α-synuclein production will help in the reduction of α-synuclein aggregate formation and improve neuronal function. This will also lead to prevention of dopaminergic cell loss and/or dysfunction. Further, extended inhibition of SNCA will reduce established aggregate pathology and will prevent dopamine neuron loss.

### Example 9

### FANA-ASOS Will Transiently Silence SNCA

Transient knockdown of SNCA will avoid the danger of any potential permanent defects that can be brought about by permanent knockdown, thus avoiding another problem common to gene knockout strategies.

### Example 10

### FANA-ASO Mediated Knockdown of Brain α-Synuclein Levels After Intracerebroventricular Administration

C57B16/C3H mice were treated with FANA-ASO (syn3) targeting α-synuclein via a single i.c.v. injection. Mice received either 94 μg or 190 μg total FANA-ASO in 5 μL PBS using a 32-gauge Hamilton syringe connected to a Neurostar digital injection unit. Untreated mice were used as a control. Mice (n=4-6 per arm) were sacrificed 4 weeks later. Brains were harvested, and the injected hemisphere homogenized in RIPA buffer containing protease inhibitors. Equal quantities of each homogenate (representing 4.8 mg wet tissue weight) were separated by SDS-PAGE (4-20%), transferred onto a nitrocellulose membrane, and immunoblotted using mAb Syn9027 (recognizing α-synuclein). Relative quantities of α-synuclein are shown in the graph (circles=untreated; squares=94 μg FANA-ASO; triangles=190 μg FANA-ASO).

The brain α-synuclein concentrations achieved following knockdown using the FANA-ASOs described are comparable to the α-synuclein concentrations present in hemizygous α-synuclein knock-out mice. Previous studies in mice have shown that this level of reduction of brain α-synuclein levels by genetic means provides significant protection against the accumulation of α-synucleinopathy in the brain and also its consequent behavioral effects. Previous in vitro and in vivo studies have also shown that ASO-mediated reduction in α-synuclein levels also reduces the accumulation of α-synucleinopathy in cultured neurons and in vivo. The FANA-ASO's described here achieved similar knockdown at 94-190 μg/animal of FANA-ASOs, a dosage that is lower than the dose used in other studies ˜750 μg/animal.

It is predicted that this magnitude of α-synuclein reduction will be beneficial in treating α-synucleinopathies (i.e. Parkinson's disease, Dementia with Lewy Bodies, Multiple System Atrophy) by slowing the accumulation of misfolded and/or toxic forms of α-synuclein and thereby attenuating neuronal dysfunction and toxicity. Reduction in brain α-synuclein levels are also expected to slow the progression of these disorders (e.g. the onset of new motor, cognitive, or autonomic symptoms) by decreasing the efficiency of cell-to-cell transmission of α-synucleinopathy to previously unaffected areas of the nervous system. Since approximately half of Alzheimer's disease patients have detectable Lewy pathology and such pathology correlates with more severe symptoms, it is expected that FANA-ASO reduction of α-synuclein levels would also provide benefits in this condition.

### Example 11

### Effect of α-Synuclein Targeting FANA-ASOs in Animal Models

In order to further demonstrate that FANA-ASO mediated α-synuclein knockdown will provide neuroprotection in synucleinopathies, α-synuclein-targeting FANA-ASOs will be tested in established animal models of α-synucleinopathy, such as the α-synuclein preformed fibril model in which recombinant fibrils are stereotaxically inoculated into the brains of wildtype mice to seed Lewy-like pathology.

To initiate pathology, wildtype (C57B16/C3H procured from Charles River Laboratories) mice stereotaxically will be injected with preformed fibrils (PFFs) assembled from wildtype mouse α-synuclein. PFFs (5 mg/mL) will be diluted to 2 mg/mL in sterile PBS in a 1.5 mL Eppendorf tube and sonicated using a Bioruptor Plus at high power for 10 cycles (30 sec on, 30 sec off) set at 10° C. A total of 2.5 μL of sonicated PFFs were stereotaxically will be injected into the dorsal striatum of 2-3 month old mice under anesthesia (ketamine/xylazine/acepromazine (60-100 mg/kg; 8-12 mg/kg; 0.5-2 mg/kg) administered i.p.). A motorized stereotaxic apparatus (Kopf Instruments) and microinjector (NeuroStar) will be connected to a 32-gauge 10 μL Hamilton syringe filled with the inoculum and targeted to the following co-ordinates (anterior/posterior relative to bregma: 10.2 mm, lateral: 2.0 mm, depth: 2.6 mm) at a rate of 0.4 μL/min. After injection, the scalp will be closed by nylon stiches and mice were provided with a 1 mL bolus of warm saline (s.c.) and allowed to recover under a warming lamp before being returned to their cages. All mice will receive a single unilateral PFF injection.

One week after PFF injection, mice will be treated with FANA-ASOs (targeting either α-synuclein or a scrambled control sequence). Mice will be anesthetized as above, and FANA-ASOs (0, 94, 190, 380 or 750 μg diluted in 5 μL PBS; n<6 animals per arm) will be administered by intracerebroventricular (i.c.v.) injection using a motorized stereotaxic apparatus and microinjector at a rate of 0.5 μL/min. Co-ordinates used for i.c.v. injections (anterior/posterior relative to bregma: +0.3 mm, lateral 1.0 mm, depth: 3.0 mm). After injection, the scalp will be closed with a surgical glue (Vetbond) and mice provided with a 1 mL bolus of warm saline (s.c.) and allowed to recover under a warming lamp. Treated mice will be returned to their cages and provided with food and water ad libitum and kept on a 12 h dark/light cycle. A subset of mice will be administered a second dose of FANA-ASOs 3 months after PFF-injection.

To determine the effect of FANA-ASO targeting α-synuclein in reducing α-synucleinopathy and protection of midbrain dopaminergic neurons, mice will be assessed for their motor performance prior to sacrifice at either 3 or 6 months after PFF-injection. Mouse all-limb grip strength will be measured using the animal grip strength test (IITC 2200). For this test a grid will be attached to a digital force transducer. Mice will be moved to a quiet behavioral testing suite and allowed to acclimate for 1 h. Each mouse will be held by the base of the tail and allowed to grasp the grid with all limbs. The maximum grip strength of 5 tests will be recorded and the average of all 5 measures reported. An accelerating rotarod (MED-Associates) will be used to assess motor coordination. Mice will receive two training sessions and two tests sessions. During the training sessions, mice will be placed on a still rod. The rod will then begin to accelerate from 4 rotations per minute (rpm) to 40 rpm over 5 min. Mice will be allowed to rest at least one hour between training and testing sessions. During the testing sessions, mice will be treated as before, and the latency to fall recorded. The trial will also be concluded if a mouse gripped the rod and rotated with it instead of walking. Mice will be allowed a maximum of 10 min on the rod.

Mice will be sacrificed by transcardial perfusion with saline, followed by 4% paraformaldehyde in PBS. Brains will be removed after craniotomy, post-fixed at 4° C. overnight and embedded in paraffin for sectioning. After perfusion and fixation, brains will be embedded in paraffin blocks, cut into 6 μm sections and mounted on glass slides. Slides will then then be stained using standard immunohistochemistry as described below. Slides will be de-paraffinized with 2 sequential 5-min washes in xylenes, followed by 1-min washes in a descending series of ethanols: 100%, 100%, 95%, 80%, 70%. Slides will then be incubated in deionized water for one minute prior to antigen retrieval as noted. After antigen retrieval, slides will be incubated in 5% hydrogen peroxide in methanol to quench endogenous peroxidase activity. Slides will be washed for 10 min in running tap water, 5 min in 0.1 M Tris, then blocked in 0.1 M Tris/2% fetal bovine serum (FBS). Slides will be incubated in primary antibodies overnight. The following primary antibodies will be used. For misfolded α-synuclein, mAb Syn506 will be used at 0.4 g/mL final concentration with microwave antigen retrieval (95° C. for 15 min with citric acid based antigen unmasking solution (Vector H-3300). To stain midbrain dopaminergic neurons, Tyrosine hydroxylase (TH-16) will be used at 1:5000 with formic acid antigen retrieval. Primary antibody will be rinsed off with 0.1 M Tris for 5 min, then incubated with goat anti-rabbit (Vector Cat #BA1000, RRID:AB_2313606) or horse anti-mouse (Vector Cat #BA2000, RRID:AB_2313581) biotinylated IgG in 0.1 M Tris/2% FBS 1:1000 for 1 h. Biotinylated antibody will be rinsed off with 0.1 M Tris for 5 min, then incubated with avidin-biotin solution (Vector Cat #PK-6100, RRID:AB_2336819) for 1 h. Slides will then be rinsed for 5 min with 0.1 M Tris, developed with ImmPACT DAB peroxidase substrate (Vector Cat #SK-4105, RRID:AB_2336520) and counterstained briefly with Harris Hematoxylin (Fisher Cat #67-650-01). Slides will be washed in running tap water for 5 min, dehydrated in ascending ethanol for 1 min each: 70%, 80%, 95%, 100%, 100%, then washed twice in xylenes for 5 min and coversliped in Cytoseal Mounting Media (Fisher Cat #23-244-256). Slides were then digitized for quantitative pathology using a Perkin-Elmer Lamina.

For analysis, section selection, annotation and quantification will be done blinded to treatment group. All quantitation will be performed in HALO quantitative pathology software (Indica Labs). Every 10th slide through the midbrain will be stained with tyrosine hydroxylase (TH). TH-stained sections will be used to annotate the substantia nigra (SN), and cell counting performed manually in a blinded manner for all sections. The sum of all sections will be multiplied by 10 to estimate the total count that would be obtained by counting every section. The SN annotations drawn onto the TH-stained sections will then be transferred to sequential sections that had been stained for misfolded α-synuclein (mAb Syn506). Amygdala regions will also be annotated on every 10th section through the length of the amygdala. A single analysis algorithm will then be applied equally to all stained sections to quantify the percentage of area occupied by Syn506 staining. Specifically, the analysis will include all DAB signal that is above threshold, which will be empirically determined to not include any background signal. This signal will then be normalized to the total tissue area.

It has been shown that mice treated with α-synuclein FANA-ASO administered via i.c.v. injection show reduced the levels of α-synuclein in the brain that is dose-dependent. In contrast, FANA-ASO containing a scrambled sequence (negative control) will show unchanged α-synuclein levels. It is expected, that at 3 months post-injection, PFF-injected mice treated with scrambled FANA-ASO will show a deterioration in grip strength and rotorod test performance compared to age-matched control animals not injected with PFFs. This motor impairment is further enhanced in the cohort allowed to survive 6 months post-injection with PFFs, correlating with additional pathology accumulation and neurodegeneration in the brain. However, treatment with α-synuclein FANA-ASO is expected to ameliorate these motor deficits in a dose-dependent manner, so that animals with the highest doses of α-synuclein FANA-ASO show the most improvement. In the 6 months post-injection cohort, it is also expected that mice that received two FANA-ASO injections will perform more favorably than those that received only one injection.

At the histological level, PFF-injected mice are expected to show α-synucleinopathy (i.e., intraneuronal inclusions containing misfolded α-synuclein) in the SN and other brain regions (e.g., amygdala, frontal cortex) at the 3-month post-injection time point. Compared to mice treatment with scrambled FANA-ASO, treatment with α-synuclein FANA-ASO is expected to reduce the pathology as measured by the proportion of tissue area occupied by mAb Syn506 immunoreactivity in both brain hemispheres. This reduction in pathology is proportional to the dose of α-synuclein FANA-ASO administered so that the highest α-synuclein FANA-ASO dosage corresponds to the least amount of pathology detected.

It is expected that at 6 months post-injection, mice treated with control FANA-ASO will show a ˜30-45% loss of TH-positive (i.e., dopaminergic) neurons in the SN on the ipsilateral side due to the accumulation of α-synucleinopathy in these cells. In cohorts that were treated with α-synuclein FANA-ASO, TH-positive cell loss in the SN is attenuated in a dose-dependent manner. Moreover, mice that received two doses of α-synuclein FANA-ASO are expected to preserve a higher number of TH-positive neurons. Similarly, TH immunoreactivity in the striatum within the hemisphere ipsilateral to PFF injection is expected to be decreased in mice treated with scrambled FANA-ASO but preserved in α-synuclein FANA-ASO treated mice in a dose-dependent manner.

Collectively, these results would indicate that the reduction of α-synuclein levels achieved by α-synuclein FANA-ASOs result in a decrease in α-synucleinopathy in an in vivo model of PD, and this consequently leads to the protection of PD-relevant cell populations such as SN dopaminergic neurons.

Although the invention has been described with reference to the above examples, it will be understood that modifications and variations are encompassed within the spirit and scope of the invention. Accordingly, the invention is limited only by the following claims.

1. A composition comprising an α-synuclein targeting FANA-ASO oligonucleotide.

2. The composition of claim 1, wherein the α-synuclein targeting FANA-ASO oligonucleotide has a nucleic acid sequence selected from the group consisting of SEQ ID NOs: 1-536 or a combination thereof.

3. The composition of claim 1, wherein the α-synuclein targeting FANA-ASO oligonucleotide comprises at least one 2′FANA modified nucleotide.

4. The composition of claim 3, wherein the at least one 2′FANA modified nucleotide is positioned within the oligonucleotide according to any of Formula 1-16.

5. A pharmaceutical composition comprising an α-synuclein targeting FANA-ASO oligonucleotide and a pharmaceutically acceptable carrier.

6. The pharmaceutical composition of claim 5, wherein the α-synuclein targeting FANA-ASO oligonucleotide has a nucleic acid sequence selected from the group consisting of SEQ ID NOs: 1-536 or a combination thereof.

7. The pharmaceutical composition of claim 5, wherein the α-synuclein targeting FANA-ASO molecule comprises at least one 2′FANA modified nucleotide.

8. The pharmaceutical composition of claim 7, wherein the at least one 2′FANA modified nucleotide is positioned within the oligonucleotide according to any of Formula 1-16.

9. The pharmaceutical composition of claim 5, wherein the pharmaceutically acceptable carrier is selected from the group consisting of phosphate buffer; citrate buffer; ascorbic acid; methionine; octadecyldimethylbenzyl ammonium chloride; hexamethonium chloride; benzalkonium chloride; benzethonium chloride; phenol alcohol; butyl alcohol; benzyl alcohol; methyl paraben; propyl paraben; catechol; resorcinol; cyclohexanol; 3-pentanol; m-cresol; low molecular weight (less than about 10 residues) polypeptides; serum albumin; gelatin; immunoglobulins; polyvinylpyrrolidone glycine; glutamine; asparagine; histidine; arginine; lysine; monosaccharides; disaccharides; glucose; mannose; dextrins; EDTA; sucrose; mannitol; trehalose; sorbitol; sodium; saline; metal surfactants; non-ionic surfactants; polyethylene glycol (PEG); magnesium stearate; water; alcohol; saline solution; glycol; mineral oil and dimethyl sulfoxide (DMSO).

10. A method of decreasing α-synuclein expression comprising administering an α-synuclein targeting FANA-ASO oligonucleotide to a subject in need thereof, thereby reducing α-synuclein expression.

11. The method of claim 10, wherein the α-synuclein expression is decreased in neurons, oligodendrocytes and/astrocytes.

12. The method of claim 10, wherein the α-synuclein targeting FANA-ASO oligonucleotide comprises at least one 2′FANA modified nucleotide.

13. The method of claim 12, wherein the at least one 2′FANA modified nucleotide is positioned within the oligonucleotide according to any of Formula 1-16.

14. The method of claim 10, wherein the α-synuclein targeting FANA-ASO oligonucleotide has a nucleic acid sequence selected from the group consisting of SEQ ID NOs: 1-536 or a combination thereof.

15. The method of claim 10, wherein the α-synuclein targeting FANA-ASO oligonucleotide has the nucleic acid sequence of SEQ ID NO:525 or SEQ ID NO:527.

16. The method of claim 10, wherein the α-synuclein targeting FANA-ASO oligonucleotide is administered by intracutaneous, subcutaneous, intravenous, intraperitoneal, intraarterial, intrathecal, intracapsular, intraorbital, intracardiac, intradermal, transdermal, transtracheal, subcuticular, intraarticular, intracerebroventricular, subcapsular, subarachnoid, intraspinal and intrasternal, oral, sublingual buccal, rectal, vaginal, ocular, infusion, inhalation, or nebulization administration.

17-35. (canceled)
